(19) United States
(12) Patent Application Publication
(10) Pub. No.: US 2024/0082394 A1
(43) Pub. Date: Mar. 14, 2024

(54) COMBINATION THERAPY FOR THE TREATMENT OF CANCER

(71) Applicant: BICARA THERAPEUTICS INC., CAMBRIDGE, MA (US)

(72) Inventors: Shivakumar Bhadravathi MARIGOWDA, SHIVAMOGA (IN); Madhukara A R, YELAHANKA NEW -TOWN (IN); Prashant Kumar PANDEY, UTTAR PRADESH (IN); Srinivas Reddy BOREDDY, BANGALORE (IN); Pradip NAIR, BANGALORE (IN)

(21) Appl. No.: 18/256,761

(22) PCT Filed: Dec. 15, 2021
(86) PCT No.: PCT/US2021/010067

§ 371 Date: Jun. 9, 2023

(30) Foreign Application Priority Data
Dec. 15, 2020 (IN) 202011054571

(51) Int. Cl. A61K 39/395; A61K 38/17; A61P 35/00

(52) U.S. Cl. CPC A61K 39/3955; A61K 38/179; A61P 35/00; A61K 2039/505

(57) ABSTRACT

Provided herein are methods of treating cancer by administering to a subject having cancer an antibody, or functional fragment or functional variant thereof, that specifically binds programmed cell death protein 1 (PD1); and a fusion protein that comprises a targeting moiety and an immunomodulatory moiety, wherein: i) said targeting moiety specifically binds epidermal growth factor receptor (EGFR); and (ii) said immunomodulatory moiety comprises an amino acid sequence of the extracellular domain of transforming growth factor-beta receptor II (TGFβRII).

### CROSS-REFERENCE TO RELATED APPLICATIONS

This application claims the priority of Indian Provisional Application No. IN202011054571, filed on Dec. 15, 2020, the entire disclosure of which is incorporated herein by reference.

### BACKGROUND

Immune checkpoint inhibitors are a class of cancer therapeutics that function to reverse T cell inhibition and tumor immunoevasion. Immune checkpoint inhibitors include, antibodies that specifically bind to and inhibit immune checkpoint proteins such as programmed cell death protein 1 (PD1) or its ligand programmed cell death-ligand 1 (PDL1) and cytotoxic T lymphocyte associated antigen 4 (CTLA4). However, antibody immune checkpoint inhibitors remain associated with several clinical problems in terms of efficacy and patient-to-patient variability. Combination therapies targeting multiple non-redundant pathways regulating immune responses may enhance immune checkpoint inhibitor efficacy. However, not all combinations provide a synergistic effect over the monotherapy components. Therefore, there is a need for combination therapies with an acceptable safety profile and high efficacy that enhance antitumor immune responses compared to monotherapy and other immunotherapy combinations.

### SUMMARY

Provided herein are methods of treating cancer in a subject that comprise administering an agent that specifically binds PD1 in combination with a fusion protein that specifically binds EGFR and binds TGFβ (e.g., fusion proteins described herein). The combination treatments disclosed herein can be particularly useful in the treatment of EGFR driven cancers.

Accordingly, in one aspect the instant disclosure provides a method of treating cancer in a human subject in need thereof, said method comprising: administering to said subject an antibody, or functional fragment or functional variant thereof, that specifically binds programmed cell death protein 1 (PD1); and administering to said subject a fusion protein that comprises a targeting moiety and an immunomodulatory moiety, wherein: i) said targeting moiety specifically binds epidermal growth factor receptor (EGFR); and (ii) said immunomodulatory moiety comprises an amino acid sequence of the extracellular domain of transforming growth factor-beta receptor II (TGFβRII).

In some embodiments, said antibody, or functional fragment or functional variant thereof, that specifically binds PD1 is a full-length antibody, a single chain variable fragment (scFv), a scFv2, a scFv-Fc, a Fab, a Fab′, a F(ab′)2, or a F(v).

In some embodiments, said antibody, or functional fragment or functional variant thereof, that specifically binds PD1 inhibits binding of PD1 to PDL1. In some embodiments, said antibody, or functional fragment or functional variant thereof, that specifically binds PD1 inhibits signaling of PD1.

In some embodiments, said antibody, or functional fragment or functional variant thereof, that specifically binds PD1 comprises a VH that comprises VH CDR1, VH CDR2, and VH CDR3, wherein VH CDR1 comprises an amino acid sequence at least 95%, 96%, 97%, 98%, 99%, or 100% identical to the amino acid sequence of SEQ ID NO: 1; VH CDR2 comprises an amino acid sequence at least 95%, 96%, 97%, 98%, 99%, or 100% identical to the amino acid sequence of SEQ ID NO: 2; and VH CDR3 comprises an amino acid sequence at least 95%, 96%, 97%, 98%, 99%, or 100% identical to the amino acid sequence of SEQ ID NO: 3.

In some embodiments, said antibody, or functional fragment or functional variant thereof, that specifically binds PD1 comprises a VL that comprises a VL CDR1, a VL CDR2, and a VL CDR3, wherein VL CDR1 comprises an amino acid sequence at least the amino acid sequence of 95%, 96%, 97%, 98%, 99%, or 100% identical to SEQ ID NO: 4; VL CDR2 comprises an amino acid sequence at least the amino acid sequence of 95%, 96%, 97%, 98%, 99%, or 100% identical to SEQ ID NO: 5; and VL CDR3 comprises an amino acid sequence at least 95%, 96%, 97%, 98%, 99%, or 100% identical to the amino acid sequence of SEQ ID NO: 6.

In some embodiments, said antibody, or functional fragment or functional variant thereof, that specifically binds PD1 comprises a VH that comprises an amino acid sequence at least 95%, 96%, 97%, 98%, 99%, or 100% identical to the amino acid sequence of SEQ ID NO: 7.

In some embodiments, said antibody, or functional fragment or functional variant thereof, that specifically binds PD1 comprises a VL that comprises an amino acid sequence at least 95%, 96%, 97%, 98%, 99%, or 100% identical to the amino acid sequence of SEQ ID NO: 8.

In some embodiments, said antibody, or functional fragment or functional variant thereof, that specifically binds PD1 comprises a heavy chain region that comprises an amino acid sequence at least 95%, 96%, 97%, 98%, 99%, or 100% identical to the amino acid sequence of SEQ ID NO: 9.

In some embodiments, said antibody, or functional fragment or functional variant thereof, that specifically binds PD1 comprises a heavy chain region that comprises an amino acid sequence at least 95%, 96%, 97%, 98%, 99%, or 100% identical to the amino acid sequence of SEQ ID NO: 10.

In some embodiments, said antibody, or functional fragment or functional variant thereof, that specifically binds PD1 comprises a light chain region that comprises an amino acid sequence at least 95%, 96%, 97%, 98%, 99%, or 100% identical to the amino acid sequence of SEQ ID NO: 11.

In some embodiments, said antibody, or functional fragment or functional variant thereof, that specifically binds PD1 comprises pembrolizumab, nivolumab, cemiplimab, spartalizumab, camrelizumab, tislelizumab, dostarlimab, cetrelimab, pidilizumab, MEDI0680, SSI-361, AMP-224, PDR001, PF-06801591, BGB-A317, TSR-042, AGEN-2034, A-0001, BGB-108, BI-754091, CBT-501, ENUM-003, ENUM-388D4, IBI-308, JNJ-63723283, JS-001, JTX-4014, JY-034, CLA-134, STIA-1110, 244C8, and 388D4, or a functional fragment or functional variant of any of the foregoing.

In some embodiments, said antibody, or functional fragment or functional variant thereof, that specifically binds PD1 comprises pembrolizumab, or a functional fragment or functional variant of any of the foregoing.

In some embodiments, said targeting moiety that specifically binds EGFR comprises an antibody or functional fragment or functional variant thereof, that specifically binds EGFR. In some embodiments, said antibody, or functional fragment or functional variant thereof, that specifically binds EGFR is a full-length antibody, a single chain variable fragment (scFv), a scFv2, a scFv-Fc, a Fab, a Fab′, a F(ab′)2, or a F(v).

In some embodiments, said antibody, or functional fragment or functional variant thereof, that specifically binds EGFR comprises a VH that comprises VH CDR1, VH CDR2, and VH CDR3, wherein VH CDR1 comprises an amino acid sequence at least 95%, 96%, 97%, 98%, 99%, or 100% identical to the amino acid sequence of SEQ ID NO: 34; VH CDR2 comprises an amino acid sequence at least 95%, 96%, 97%, 98%, 99%, or 100% identical to the amino acid sequence of SEQ ID NO: 35; and VH CDR3 comprises an amino acid sequence at least 95%, 96%, 97%, 98%, 99%, or 100% identical to the amino acid sequence of SEQ ID NO: 36.

In some embodiments, said antibody, or functional fragment or functional variant thereof, that specifically binds EGFR comprises a VL that comprises a VL CDR1, a VL CDR2, and a VL CDR3, wherein VL CDR1 comprises an amino acid sequence at least 95%, 96%, 97%, 98%, 99%, or 100% identical to the amino acid sequence of SEQ ID NO: 37; VL CDR2 comprises an amino acid sequence at least 95%, 96%, 97%, 98%, 99%, or 100% identical to the amino acid sequence of SEQ ID NO: 38; and VL CDR3 comprises an amino acid sequence at least 95%, 96%, 97%, 98%, 99%, or 100% identical to the amino acid sequence of SEQ ID NO: 39.

In some embodiments, said antibody, or functional fragment or functional variant thereof, that specifically binds EGFR comprises a VH that comprises an amino acid sequence at least 95%, 96%, 97%, 98%, 99%, or 100% identical to the amino acid sequence of SEQ ID NO: 40.

In some embodiments, said antibody, or functional fragment or functional variant thereof, that specifically binds EGFR comprises a VL that comprises an amino acid sequence at least 95%, 96%, 97%, 98%, 99%, or 100% identical to the amino acid sequence of SEQ ID NO: 41.

In some embodiments, said antibody, or functional fragment or functional variant thereof, that specifically binds EGFR comprises a heavy chain that comprises an amino acid sequence at least 95%, 96%, 97%, 98%, 99%, or 100% identical to the amino acid sequence of SEQ ID NO: 42.

In some embodiments, said antibody, or functional fragment or functional variant thereof, that specifically binds EGFR consists of a heavy chain that comprises an amino acid sequence at least 95%, 96%, 97%, 98%, 99%, or 100% identical to the amino acid sequence of SEQ ID NO: 43.

In some embodiments, said antibody, or functional fragment or functional variant thereof, that specifically binds EGFR comprises a heavy chain that consists of an amino acid sequence at least 95%, 96%, 97%, 98%, 99%, or 100% identical to the amino acid sequence of SEQ ID NO: 42.

In some embodiments, said antibody, or functional fragment or functional variant thereof, that specifically binds EGFR consists of a heavy chain that consists of an amino acid sequence at least 95%, 96%, 97%, 98%, 99%, or 100% identical to the amino acid sequence of SEQ ID NO: 43.

In some embodiments, said antibody, or functional fragment or functional variant thereof, that specifically binds EGFR comprises a light chain that comprises an amino acid sequence at least 95%, 96%, 97%, 98%, 99%, or 100% identical to the amino acid sequence of SEQ ID NO: 44.

In some embodiments, said antibody, or functional fragment or functional variant thereof, that specifically binds EGFR consists of a light chain that consists of an amino acid sequence at least 95%, 96%, 97%, 98%, 99%, or 100% identical to the amino acid sequence of SEQ ID NO: 44.

In some embodiments, said antibody, or functional fragment or functional variant thereof, that specifically binds EGFR comprises cetuximab or panitumumab, or a functional fragment or functional variant of any of the foregoing.

In some embodiments, said immunomodulatory moiety comprises an amino acid sequence at least 95%, 96%, 97%, 98%, 99%, or 100% identical to SEQ ID NO: 56. In some embodiments, said immunomodulatory moiety consists of an amino acid sequence at least 95%, 96%, 97%, 98%, 99%, or 100% identical to the amino acid sequence of SEQ ID NO: 56.

In some embodiments, said immunomodulatory moiety is indirectly fused to said targeting moiety. In some embodiments, said immunomodulatory moiety is indirectly fused to said targeting moiety via a peptide linker. In some embodiments, said immunomodulatory moiety is indirectly fused to said targeting moiety via a peptide linker of sufficient length such that said immunomodulatory moiety and said targeting moiety can simultaneously bind the respective targets.

In some embodiments, said linker comprises the amino acid sequence of SEQ ID NO: 57, 58, 59, 60, or 61. In some embodiments, said linker comprises the amino acid sequence of SEQ ID NO: 57. In some embodiments, said linker consists of the amino acid sequence of SEQ ID NO: 57.

In some embodiments, said immunomodulatory moiety is fused to the C terminus of said targeting moiety. In some embodiments, said immunomodulatory moiety is fused to the N terminus of said targeting moiety.

In some embodiments, said targeting moiety is an antibody that comprises a light chain and a heavy chain, and wherein said immunomodulatory moiety is fused to the C terminus of said heavy chain of said targeting moiety. In some embodiments, said targeting moiety is an antibody that comprises a light chain and a heavy chain, and wherein said immunomodulatory moiety is fused to the C terminus of said light chain of said targeting moiety.

In some embodiments, said targeting moiety is an antibody specifically binds epidermal growth factor receptor (EGFR) that comprises a heavy chain that comprises an amino acid sequence at least 95%, 96%, 97%, 98%, 99%, or 100% identical to the amino acid sequence of SEQ ID NO: 43, and a light chain that comprises an amino acid sequence at least 95%, 96%, 97%, 98%, 99%, or 100% identical to the amino acid sequence of SEQ ID NO: 44, and wherein said immunomodulatory moiety comprises an amino acid sequence at least 95%, 96%, 97%, 98%, 99%, or 100% identical to the amino acid sequence of SEQ ID NO: 56, and wherein the N terminus of said immunomodulatory moiety is fused indirectly through a linker to the C terminus of said heavy chain or said light chain, and wherein said linker comprises an amino acid sequence at least 95%, 96%, 97%, 98%, 99%, or 100% identical to the amino acid sequence of SEQ ID NO: 57.

In some embodiments, said targeting moiety is an antibody specifically binds epidermal growth factor receptor (EGFR) that comprises a heavy chain that comprises an amino acid sequence at least 95%, 96%, 97%, 98%, 99%, or 100% identical to the amino acid sequence of SEQ ID NO: 43, and a light chain that comprises an amino acid sequence at least 95%, 96%, 97%, 98%, 99%, or 100% identical to the amino acid sequence of SEQ ID NO: 44, and wherein said immunomodulatory moiety comprises an amino acid sequence at least 95%, 96%, 97%, 98%, 99%, or 100% identical to the amino acid sequence of SEQ ID NO: 56, and wherein the N terminus of said immunomodulatory moiety is fused indirectly through a linker to the C terminus of said light chain, and wherein said linker comprises an amino acid sequence at least 95%, 96%, 97%, 98%, 99%, or 100% identical to the amino acid sequence of SEQ ID NO: 57.

In some embodiments, said targeting moiety comprises an antibody that comprises a heavy chain comprising an amino acid sequence at least 95%, 96%, 97%, 98%, 99%, or 100% identical to the amino acid sequence of SEQ ID NO: 43; and a light chain comprising an amino acid sequence at least 95%, 96%, 97%, 98%, 99%, or 100% identical to the amino acid sequence of SEQ ID NO: 62.

In some embodiments, said cancer is a solid tumor. In some embodiments, said cancer is selected from the group consisting of breast cancer, anal cancer, pancreatic cancer, thyroid cancer, liver cancer, ovarian cancer, lung cancer, skin cancer, brain cancer, spinal cord cancer, head cancer, neck cancer, and head and neck cancer.

In some embodiments, said cancer is head and neck cancer. In some embodiments, said cancer is head and neck squamous cell carcinoma (HNSCC). In some embodiments, said cancer is recurrent HNSCC. In some embodiments, said cancer is metastatic HNSCC. In some embodiments, said cancer is recurrent and metastatic HNSCC.

In some embodiments, said cancer is squamous cell carcinoma of anal canal (SCCAC). In some embodiments, said cancer is recurrent SCCAC. In some embodiments, said cancer is metastatic SCCAC. In some embodiments, said cancer is recurrent and metastatic SCCAC.

In some embodiments, said antibody, or functional fragment or functional variant thereof, that specifically binds PD1 is administered to said human subject at a dose from about 100 mg to 500 mg, 100 mg to 400 mg, 100 mg to 300 mg, or 100 mg to 200 mg.

In some embodiments, said antibody, or functional fragment or functional variant thereof, that specifically binds PD1 is administered to said human subject at a dose of about 100 mg, 200 mg, 300 mg, 400 mg, or 500 mg. In some embodiments, said antibody, or functional fragment or functional variant thereof, that specifically binds PD1 is administered to said human subject at a dose of about 200 mg. In some embodiments, said antibody, or functional fragment or functional variant thereof, that specifically binds PD1 is administered to said human subject at a dose of about 300 mg.

In some embodiments, said antibody, or functional fragment or functional variant thereof, that specifically binds PD1 is administered to said human subject every 1, 2, 3, or 4 weeks. In some embodiments, said antibody, or functional fragment or functional variant thereof, that specifically binds PD1 is administered to said human subject every 3 weeks.

In some embodiments, said fusion protein is administered to said human subject at a dose from about 50 mg to 2000 mg, 100 mg to 2000 mg, 150 mg to 2000 mg, 200 mg to 2000 mg, 300 mg to 2000 mg, 400 mg to 2000 mg, 500 mg to 2000 mg, 600 mg to 2000 mg, 700 mg to 2000 mg, 800 mg to 2000 mg, 9000 mg to 2000 mg, 1000 mg to 2000 mg, 1500 mg to 2000 mg, 50 mg to 100 mg, 50 mg to 500 mg, 50 mg to 400 mg, 50 mg to 300 mg, 50 mg to 200 mg, 50 mg to 100 mg, 100 mg to 500 mg, 100 mg to 400 mg, 100 mg to 300 mg, or 100 mg to 200 mg. In some embodiments, said fusion protein is administered to said human subject at a dose from about 200 mg to 2000 mg. In some embodiments, said fusion protein is administered to said human subject at a dose of about 50 mg, 60 mg, 64 mg, 100 mg, 150 mg, 200 mg, 240 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900, or 2000 mg. In some embodiments, said fusion protein is administered to said human subject at a dose of about 64 mg, 240 mg, 800 mg, or 1600 mg.

In some embodiments, said fusion protein is administered to said human subject every 1, 2, 3, or 4 weeks. In some embodiments, said fusion protein is administered to said human subject every week. In some embodiments, said fusion protein is administered to said human subject 3 weeks.

In some embodiments, said fusion protein is co-administered, administered prior to, or administered after, said antibody, or functional fragment or functional variant thereof, that specifically binds PD1.

### BRIEF DESCRIPTION OF THE FIGURES

FIG. 1 is a line graph that shows the effect of cetuximab, BCA101(Fusion mAb anti EGFR+TGFβRII ECD), anti-PD1 (pembrolizumab), and BCA101+anti-PD1 (pembrolizumab) in huNOG-EXL mice bearing PC-3 xenograft tumor on tumor volume over the course of 21 days. Values are expressed as Mean±SEM of 8-10 animals in each group. Statistical analysis carried out by Two-way ANOVA followed by Bonferroni post tests using Graph Pad Prism (Version 8.3.0). *** significant (p<0.001) difference when respective treatment groups were compared with isotype control group on day 18. ^{SSS}significant (p<0.001) and ^{#}significant (p<0.05) difference when combination treatment group was compared with BCA101 and pembrolizumab respectively on day 18.

FIGS. 2A-2E are line graphs that show the individual tumor growth curve of huNOG-EXL mice PC-3 tumor xenograft over the course of 21 days. FIG. 2A is a line graph that shows the individual tumor growth in the isotype control group over 21 days. FIG. 2B is a line graph that shows the individual tumor growth in the cetuximab treatment group over 21 days. FIG. 2C is a line graph that shows the individual tumor growth in the BCA101 treatment group over 21 days. FIG. 2D is a line graph that shows the individual tumor growth in the anti-PD1 (pembrolizumab) treatment group over 21 days. FIG. 2E is a line graph that shows the individual tumor growth in the BCA101+anti-PD1 (pembrolizumab) treatment group over 21 days.

FIG. 3A shows photographs of huNOG-EXL mice bearing PC-3 tumors, from the control group (isotype control), the cetuximab treatment group, and the BCA101 treatment group. Photograph were captured on day 19 of the study. FIG. 3B shows photographs of huNOG-EXL mice bearing PC-3 tumors, from the anti-PD1 (pembrolizumab) treatment group and the BCA101+anti-PD1 (pembrolizumab) treatment group. Photograph were captured on day 19 of the study.

FIG. 4 is a line graph that shows the effect of cetuximab, BCA101, anti-PD1 (pembrolizumab), and BCA101+anti-PD1 (pembrolizumab) on the percentage change in body weight of huNOG-EXL mice bearing PC-3 tumor xenografts. Values are expressed as Mean±SEM of 8-10 animals in each group. There was gradual body weight loss in all the groups. There were no visible clinical signs or abnormal behavior in any of the treated groups.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Overview

The present disclosure provides, inter alia, new combination therapies comprising an agent that specifically binds to and inhibits the function of PD1 and an EGFR targeted immunomodulatory fusion protein that binds TGFβ. The combination therapies described herein provide a synergistic effect and improved efficacy over each of the monotherapies. In some embodiments, the EGFR fusion protein comprises a targeting moiety that specifically binds EGFR and an immunomodulatory moiety that comprises an amino acid sequence of the extracellular domain of transforming growth factor-beta receptor II (TGFβRII). The combination treatments disclosed herein can be particularly useful in the treatment of EGFR driven cancers, such as head and neck cancers and anal cancer.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise.

It must be noted that, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and/or “consisting essentially of” are also provided.

The section headings used herein are for organizational purposes only and are not to be construed as limiting the subject matter described.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “programmed cell death protein 1” and “PD1” are used interchangeably herein and refer to an immunoinhibitory receptor belonging to the CD28 family. PD1 is expressed predominantly on previously activated T cells in vivo, and binds to two ligands, PDL1 and PDL2. The term PD1 as used herein includes human PD1 (hPD1), variants, isoforms, and species homologs of hPD1, and analogs having at least one common epitope with hPD1. The complete hPD-1 sequence can be found under GenBank Accession No. U64863.

The terms “epidermal growth factor receptor” and “EGFR” are used interchangeably herein and refer to a transmembrane protein that is a receptor for members of the epidermal growth factor family (EGF family) of extracellular protein ligands. The term EGFR as used herein includes human EGFR (hEGFR), variants, isoforms, and species homologs of hEGFR, and analogs having at least one common epitope with EGFR. The complete hEGFR sequence can be found under GenBank Gene ID: 1956.

The terms “subject” and “patient” are used interchangeably herein and include any human or nonhuman animal. The term “nonhuman animal” includes, but is not limited to, vertebrates such as nonhuman primates, sheep, dogs, and rodents such as mice, rats and guinea pigs. In some embodiments, the subject is a human.

As used herein, the term “administering” refers to the physical introduction of a therapeutic agent (or a precursor of the therapeutic agent that is metabolized or altered within the body of the subject to produce the therapeutic agent in vivo) to a subject, using any of the various methods and delivery systems known to those skilled in the art. Exemplary routes of include intravenous, intramuscular, subcutaneous, intraperitoneal, spinal or other parenteral routes of administration, for example by injection or infusion. The term “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent may be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “cancer” and “tumor” are used interchangeably herein and refer to a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream.

A “therapeutically effective amount” or “therapeutically effective dose” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “weight based dose” as used herein refers to a dose that is administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration.

The term “fixed dose” as used herein refers to two or more different proteins in a single composition (e.g., anti-PD-1 antibody and fusion protein) are present in the composition in particular (fixed) ratios with each other. In some embodiments, the fixed dose is based on the weight (e.g., mg) of the proteins. In certain embodiments, the fixed dose is based on the concentration (e.g., mg/ml) of the proteins.

The term “flat dose” as used herein refers to a dose that is administered to a patient without regard for the weight or body surface area (BSA) of the patient. The flat dose is therefore not provided as a mg/kg dose, but rather as an absolute amount of the agent (e.g., the fusion protein and/or anti-PD-1 antibody). For example, a 60 kg person and a 100 kg person would receive the same dose of an antibody.

The term “antibody” is used herein in the broadest sense and encompasses fully assembled antibodies; functional antibody fragments and functional variants thereof that can bind antigen (e.g., Fab, F(ab′)2, Fv, single chain variable fragment (scFv), single domain antibodies (e.g., VHH), diabodies, antibody chimeras, hybrid antibodies, bispecific antibodies, and the like); and non-antibody fragments that bind antigen (e.g., recombinant fibronectin domains) and recombinant polypeptides comprising the forgoing. Unless otherwise specified, references to the numbering of specific amino acid residue positions in an antibody are according to the EU numbering system, as described in Kabat et al., U.S. Dept. of Health and Human Services, Sequences of Proteins of Immunological Interest (1983) (“Kabat”).

As used herein, the “variable region” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions and three complementarity determining regions.

As used herein, the term “complementarity determining region” refers to each of the regions of an antibody variable domain which are hypervariable in sequence and form structurally defined loops (“hypervariable loops”). Generally, native four-chain antibodies comprise six CDRs; three in the VH (H1, H2, H3), and three in the VL (L1, L2, L3). The CDRs have been described by Kabat et al., U.S. Dept. of Health and Human Services, Sequences of Proteins of Immunological Interest (1983) (“Kabat”) and by Chothia et al., J Mol Biol 196:901-917 (1987), where the definitions include overlapping or subsets of amino acid residues when compared against each other. Nevertheless, application of either definition to refer to a CDR of an antibody is intended to be within the scope of the term as defined and used herein. Those skilled in the art can routinely determine which residues comprise a particular CDR given the variable region amino acid sequence of the antibody. Unless otherwise specified, CDRs are defined according to the Kabat system.

The term “fusion protein” and grammatical equivalents as used herein refers to a protein that comprises an amino acid sequence derived from at least two separate proteins. The amino acid sequence of the at least two separate proteins can be directly connected through a peptide bond; or can be operably connected through an amino acid linker. Therefore, the term fusion protein encompasses embodiments, wherein the amino acid sequence of e.g., Protein A is directly connected to the amino acid sequence of Protein B through a peptide bond (Protein A— Protein B), and embodiments, wherein the amino acid sequence of e.g., Protein A is operably connected to the amino acid sequence of Protein B through an amino acid linker (Protein A— linker—Protein B).

The term “fuse” and grammatical equivalents thereof as used herein refers to the operable connection of an amino acid sequence derived from one protein to the amino acid sequence derived from different protein. The term fuse encompasses both a direct connection of the two amino acid sequences through a peptide bond, and the indirect connection through an amino acid linker.

As used herein, the term “modification,” with reference to a nucleic acid sequence, refers to a nucleic acid sequence that comprises at least one substitution, addition, or deletion of nucleotide compared to a reference nucleic acid sequence. As used herein, the term “modification,” with reference to an amino acid sequence refers to an amino acid sequence that comprises at least one substitution, addition, or deletion of an amino acid residue compared to a reference nucleic acid sequence. Naturally occurring amino acid derivatives are not considered modified amino acids for purposes of determining percent identity of two amino acid sequences. For example, a naturally occurring modification of a glutamate amino acid residue to a pyroglutamate amino acid residue would not be considered an amino acid modification for purposes of determining percent identity of two amino acid sequences. Further, for example, a naturally occurring modification of a glutamate amino acid residue to a pyroglutamate amino acid residue would not be considered an amino acid “modification” as defined herein. Modifications can include the inclusion of non-naturally occurring amino acid residues.

The term “identical” or “percent identity” with reference to a nucleic acid sequence or amino acid sequence refers to at least two nucleic acid or at least two amino acid sequences or subsequences that have a specified percentage of nucleotides or amino acids, respectively, that are the same, when compared and aligned for maximum correspondence, as measured using a sequence comparison algorithm or by visual inspection. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Examples of algorithms that are suitable for determining percent sequence identity and sequence similarity are the BLAST and BLAST 2.0 algorithms, which are described in Altschuel et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information. The percent identity between two sequences can be determined using techniques similar to those described above, with or without allowing gaps. In calculating percent identity, typically only exact matches are counted. As described above, the percent identity is based on the amino acid matches between the smaller of two proteins.

### Anti-PD1 Antibodies

In certain aspects, provided herein are methods of treating cancer comprising administering to a subject having cancer an antibody, or functional fragment or functional variant thereof, that specifically binds programmed death protein 1 (PD1), in combination with a fusion protein described herein. PD-1 is a member of the CD28 family of receptors, which includes CD28, CTLA-4, ICOS, PD-1, and BTLA. Two cell surface glycoprotein ligands for PD1 have been identified, programmed death ligand 1 (PDL1) and programmed death ligand 2 (PDL2), that are expressed on antigen-presenting cells as well as many human cancers and have been shown to down regulate T cell activation and cytokine secretion upon binding to PD1.

In some embodiments, the antibody is a full-length antibody, a single chain variable fragment (scFv), a scFv2, a scFv-Fc, a Fab, a Fab′, a F(ab′)2, a F(v), a single domain antibody, a single chain antibody, or a VHH.

Exemplary human monoclonal antibodies that bind specifically to PD1 with high affinity have been disclosed in U.S. Pat. No. 8,008,449. Each of the anti-PD-1 human monoclonal antibodies disclosed in U.S. Pat. No. 8,008,449 has been demonstrated to exhibit one or more of the following characteristics: (a) binds to human PD1 with a KD of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PDL1 and/or PDL2 to PD1; (h) stimulates antigen-specific memory responses; (i) stimulates antibody responses; and/or (j) inhibits tumor cell growth in vivo. Anti-PD1 antibodies usable in the present disclosure include monoclonal antibodies that bind specifically to human PD1 and exhibit at least one, at least two, at least three, at least four or at least five of the preceding characteristics. In other embodiments, the anti-PD-1 antibody is chosen from the human antibodies 17D8, 2D3, 4H1, 4A11, 7D3 or 5F4 described in U.S. Pat. No. 8,008,449. Other anti-PD1 monoclonal antibodies have been described in, for example, U.S. Pat. Nos. 6,808,710, 7,488,802, 8,168,757 and 8,354,509, and PCT Publication No. WO 2012/145493.

In some embodiments, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see e.g., WO2008/156712), PDR001 (Novartis; also known as spartalizumab; see e.g., WO 2015/112900), MEDI0680 (AstraZeneca; also known as AMP-514; see e.g., WO2012145493), cemiplimab (Regeneron; also known as REGN-2810; see e.g., WO2015112800), JS001 (Taizhou Junshi Pharma; see e.g., Si-Yang Liu et al., J. Hematol. Oncol. 70: 136 (2017)), BGB-A317 (Tislelizumab Beigene; see e.g., WO201535606 and US20150079109), INCSHR1210 (Jiangsu Hengrui Medicine; also known as SHR-1210; see e.g., WO2015085847; Si-Yang Liu et al., J. Hematol. Oncol. 70: 136 (2017)), TSR-042 (Tesaro Biopharmaceutical; also known as AB011; see e.g., WO2014179664), GLS-010 (Wuxi/Harbin Gloria Pharmaceuticals; also known as WBP3055; see e.g., Si-Yang Liu et al., J. Hematol. Oncol. 70: 136 (2017)), AM-0001 (Armo), STI-1110 (Sorrento Therapeutics; see e.g., WO2014194302), AGEN2034 (Agenus; see e.g., WO 2017/040790), MGA012 (Macrogenics, see e.g., WO201719846), IBI308 (Innovent; see e.g., WO2017024465, WO2017025016, WO2017132825, and WO2017133540), and BCD-100 (Biocad).

In some embodiments, the anti-PD1 antibody is selected from the group consisting of pembrolizumab, nivolumab, cemiplimab, spartalizumab, camrelizumab, tislelizumab, dostarlimab, cetrelimab, pidilizumab, MEDI0680, SSI-361, AMP-224, PDR001, PF-06801591, BGB-A317, TSR-042, AGEN-2034, A-0001, BGB-108, BI-754091, CBT-501, ENUM-003, ENUM-388D4, IBI-308, JNJ-63723283, JS-001, JTX-4014, JY-034, CLA-134, STIA-1110, 244C8, and 388D4.

In some embodiments, the anti-PD1 antibody is a functional fragment of pembrolizumab, nivolumab, spartalizumab, cemiplimab, camrelizumab, tislelizumab, dostarlimab, cetrelimab, pidilizumab, MEDI0680, SSI-361, AMP-224, PDR001, PF-06801591, BGB-A317, TSR-042, AGEN-2034, A-0001, BGB-108, BI-754091, CBT-501, ENUM-003, ENUM-388D4, IBI-308, JNJ-63723283, JS-001, JTX-4014, JY-034, CLA-134, STIA-1110, 244C8, or 388D4.

In some embodiments, the anti-PD1 antibody is a functional variant of pembrolizumab, nivolumab, spartalizumab, cemiplimab, camrelizumab, tislelizumab, dostarlimab, cetrelimab, pidilizumab, MEDI0680, SSI-361, AMP-224, PDR001, PF-06801591, BGB-A317, TSR-042, AGEN-2034, A-0001, BGB-108, BI-754091, CBT-501, ENUM-003, ENUM-388D4, IBI-308, JNJ-63723283, JS-001, JTX-4014, JY-034, CLA-134, STIA-1110, 244C8, or 388D4.

### Pembrolizumab

In some embodiments, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD1. Pembrolizumab is described, for example, in U.S. Pat. No. 8,900,587. In some embodiments, the anti-PD1 antibody cross-competes with pembrolizumab. In some embodiments, the anti-PD1 antibody binds to the same epitope as pembrolizumab. In some embodiments, the anti-PD1 antibody has the same CDRs as pembrolizumab.

In some embodiments the anti-PD1 antibody comprises a variable heavy chain (VH) that comprises three complementarity determining regions: VH CDR1, VH CDR2, and VH CDR3. In some embodiments, the anti-PD1 antibody comprises a VH comprising a VH CDR1 that comprises an amino acid sequence of SEQ ID NO: 1, with 0, 1, 2, or 3 amino acid modifications; a VH CDR2 that comprises an amino acid sequence of SEQ ID NO: 2, with 0, 1, 2, or 3 amino acid modifications; and a VH CDR3 that comprises an amino acid sequence of SEQ ID NO: 3, with 0, 1, 2, or 3 amino acid modifications.

In some embodiments, the anti-PD1 antibody comprises a VH comprising a VH CDR1 that comprises the amino acid sequence of SEQ ID NO: 1, or the amino acid sequence of SEQ ID NO: 1 with 1, 2, or 3 amino acid modifications; a VH CDR2 that comprises the amino acid sequence of SEQ ID NO: 2, or the amino acid sequence of SEQ ID NO: 2 with 1, 2, or 3 amino acid modifications; and a VH CDR3 that comprises the amino acid sequence of SEQ ID NO: 3, or the amino acid sequence of SEQ ID NO: 3 with 1, 2, or 3 amino acid modifications.

In some embodiments, the anti-PD1 antibody comprises a VL comprising a VL CDR1 that comprises an amino acid sequence of SEQ ID NO: 4, with 0, 1, 2, or 3 amino acid modifications; a VL CDR2 that comprises an amino acid sequence of SEQ ID NO: 5, with 0, 1, 2, or 3 amino acid modifications; and a VL CDR3 that comprises an amino acid sequence of SEQ ID NO: 6, with 0, 1, 2, or 3 amino acid modifications.

In some embodiments, the anti-PD1 antibody comprises a VL comprising a VL CDR1 that comprises the amino acid sequence of SEQ ID NO: 4, or the amino acid sequence of SEQ ID NO: 4 with 1, 2, or 3 amino acid modifications; a VL CDR2 that comprises the amino acid sequence of SEQ ID NO: 5, or the amino acid sequence of SEQ ID NO: 5 with 1, 2, or 3 amino acid modifications; and a VL CDR3 that comprises the amino acid sequence of SEQ ID NO: 6, or the amino acid sequence of SEQ ID NO: 6 with 1, 2, or 3 amino acid modifications.

In some embodiments, the anti-PD1 antibody comprises a VH comprising a VH CDR1 that comprises an amino acid sequence of SEQ ID NO: 1, with 0, 1, 2, or 3 amino acid modifications; a VH CDR2 that comprises an amino acid sequence of SEQ ID NO: 2, with 0, 1, 2, or 3 amino acid modifications; and a VH CDR3 that comprises an amino acid sequence of SEQ ID NO: 3, with 0, 1, 2, or 3 amino acid modifications; and the anti-PD1 antibody comprises a VL that comprises comprising a VL CDR1 that comprises an amino acid sequence of SEQ ID NO: 4, with 0, 1, 2, or 3 amino acid modifications; a VL CDR2 that comprises an amino acid sequence of SEQ ID NO: 5, with 0, 1, 2, or 3 amino acid modifications; and a VL CDR3 that comprises an amino acid sequence of SEQ ID NO: 6, with 0, 1, 2, or 3 amino acid modifications.

In some embodiments, the anti-PD1 antibody comprises a VH comprising a VH CDR1 that comprises the amino acid sequence of SEQ ID NO: 1, or the amino acid sequence of SEQ ID NO: 1 with 1, 2, or 3 amino acid modifications; a VH CDR2 that comprises the amino acid sequence of SEQ ID NO: 2, or the amino acid sequence of SEQ ID NO: 2 with 1, 2, or 3 amino acid modifications; and a VH CDR3 that comprises the amino acid sequence of SEQ ID NO: 3, or the amino acid sequence of SEQ ID NO: 3 with 1, 2, or 3 amino acid modifications; and the anti-PD1 antibody comprises a VL comprising a VL CDR1 that comprises the amino acid sequence of SEQ ID NO: 4, or the amino acid sequence of SEQ ID NO: 4 with 1, 2, or 3 amino acid modifications; a VL CDR2 that comprises the amino acid sequence of SEQ ID NO: 5, or the amino acid sequence of SEQ ID NO: 5 with 1, 2, or 3 amino acid modifications; and a VL CDR3 that comprises the amino acid sequence of SEQ ID NO: 6, or the amino acid sequence of SEQ ID NO: 6 with 1, 2, or 3 amino acid modifications.

In some embodiments, the anti-PD1 antibody comprises a VH comprising a VH CDR1 that comprises an amino acid sequence at least 95%, 96%, 97%, 98%, 99%, or 100% identical to the amino acid sequence of SEQ ID NO: 1; a VH CDR2 that comprises an amino acid sequence at least 95%, 96%, 97%, 98%, 99%, or 100% of the amino acid sequence of SEQ ID NO: 2; and a VH CDR3 that comprises an amino acid sequence at least 95%, 96%, 97%, 98%, 99%, or 100% identical of the amino acid sequence of SEQ ID NO: 3.

In some embodiments, the anti-PD1 antibody comprises a VL comprising a VL CDR1 that comprises an amino acid sequence at least 95%, 96%, 97%, 98%, 99%, or 100% identical to the amino acid sequence of SEQ ID NO: 4; a VL CDR2 that comprises an amino acid sequence at least 95%, 96%, 97%, 98%, 99%, or 100% of the amino acid sequence of SEQ ID NO: 5; and a VL CDR3 that comprises an amino acid sequence at least 95%, 96%, 97%, 98%, 99%, or 100% identical of the amino acid sequence of SEQ ID NO: 6.

In some embodiments, the anti-PD1 antibody comprises a VH comprising a VH CDR1 that comprises an amino acid sequence at least 95%, 96%, 97%, 98%, 99%, or 100% identical to the amino acid sequence of SEQ ID NO: 1; a VH CDR2 that comprises an amino acid sequence at least 95%, 96%, 97%, 98%, 99%, or 100% identical to the amino acid sequence of SEQ ID NO: 2; and a VH CDR3 that comprises an amino acid sequence at least 95%, 96%, 97%, 98%, 99%, or 100% identical to the amino acid sequence of SEQ ID NO: 3; and the anti-PD1 antibody comprises a VL comprising a VL CDR1 that comprises an amino acid sequence at least 95%, 96%, 97%, 98%, 99%, or 100% identical to the amino acid sequence of SEQ ID NO: 4; a VL CDR2 that comprises an amino acid sequence at least 95%, 96%, 97%, 98%, 99%, or 100% identical to the amino acid sequence of SEQ ID NO: 5; and a VL CDR3 that comprises an amino acid sequence at least 95%, 96%, 97%, 98%, 99%, or 100% identical to the amino acid sequence of SEQ ID NO: 6.

In some embodiments, the anti-PD1 antibody comprises a VH at least 90%, 91%, 92%, 93%, 94%, 95%, 96%, 97%, 98%, 99%, or 100% identical to the amino acid sequence of SEQ ID NO: 7. In some embodiments, the anti-PD1 antibody comprises a VL at least 90%, 91%, 92%, 93%, 94%, 95%, 96%, 97%, 98%, 99%, or 100% identical to the amino acid sequence of SEQ ID NO: 8. In some embodiments, the anti-PD1 antibody comprises a VH at least 90%, 91%, 92%, 93%, 94%, 95%, 96%, 97%, 98%, 99%, or 100% identical to the amino acid sequence of SEQ ID NO: 7; and a VL at least 90%, 91%, 92%, 93%, 94%, 95%, 96%, 97%, 98%, 99%, or 100% identical to the amino acid sequence of SEQ ID NO: 8.

In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 10. In some embodiments, the anti-PD1 antibody comprises a light chain that comprises an amino acid sequence at least 90%, 91%, 92%, 93%, 94%, 95%, 96%, 97%, 98%, 99%, or 100% identical to the amino acid sequence of SEQ ID NO: 11. In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 10; and a light chain that comprises an amino acid sequence at least 90%, 91%, 92%, 93%, 94%, 95%, 96%, 97%, 98%, 99%, or 100% identical to the amino acid sequence of SEQ ID NO: 11.

In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 9. In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 9; and a light chain that comprises an amino acid sequence at least 90%, 91%, 92%, 93%, 94%, 95%, 96%, 97%, 98%, 99%, or 100% identical to the amino acid sequence of SEQ ID NO: 11.

### Nivolumab

In some embodiments, the anti-PD1 antibody is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD1 ligands (PDL1 and PDL2), thereby blocking the down-regulation of antitumor T-cell functions (U.S. Pat. No. 8,008,449; Wang et al., 2014 Cancer Immunol Res. 2(9):846-56; referred to as 5C4 in WO 2006/121168). In some embodiments, the anti-PD1 antibody cross-competes with nivolumab. In some embodiments, the anti-PD-1 antibody binds to the same epitope as nivolumab. In some embodiments, the anti-PD-1 antibody has the same CDRs as nivolumab.

In some embodiments the anti-PD1 antibody comprises a variable heavy chain (VH) that comprises three complementarity determining regions: VH CDR1, VH CDR2, and VH CDR3. In some embodiments, the anti-PD1 antibody comprises a VH comprising a VH CDR1 that comprises an amino acid sequence of SEQ ID NO: 12, with 0, 1, 2, or 3 amino acid modifications; a VH CDR2 that comprises an amino acid sequence of SEQ ID NO: 13, with 0, 1, 2, or 3 amino acid modifications; and a VH CDR3 that comprises an amino acid sequence of SEQ ID NO: 14, with 0, 1, 2, or 3 amino acid modifications.

In some embodiments, the anti-PD1 antibody comprises a VH comprising a VH CDR1 that comprises the amino acid sequence of SEQ ID NO: 12, or the amino acid sequence of SEQ ID NO: 12 with 1, 2, or 3 amino acid modifications; a VH CDR2 that comprises the amino acid sequence of SEQ ID NO: 13, or the amino acid sequence of SEQ ID NO: 13 with 1, 2, or 3 amino acid modifications; and a VH CDR3 that comprises the amino acid sequence of SEQ ID NO: 14, or the amino acid sequence of SEQ ID NO: 14 with 1, 2, or 3 amino acid modifications.

In some embodiments, the anti-PD1 antibody comprises a VL comprising a VL CDR1 that comprises an amino acid sequence of SEQ ID NO: 15, with 0, 1, 2, or 3 amino acid modifications; a VL CDR2 that comprises an amino acid sequence of SEQ ID NO: 16, with 0, 1, 2, or 3 amino acid modifications; and a VL CDR3 that comprises an amino acid sequence of SEQ ID NO: 17, with 0, 1, 2, or 3 amino acid modifications. In some embodiments, the anti-PD1 antibody comprises a VL comprising a VL CDR1 that comprises the amino acid sequence of SEQ ID NO: 15, or the amino acid sequence of SEQ ID NO: 15 with 1, 2, or 3 amino acid modifications; a VL CDR2 that comprises the amino acid sequence of SEQ ID NO: 16, or the amino acid sequence of SEQ ID NO: 16 with 1, 2, or 3 amino acid modifications; and a VL CDR3 that comprises the amino acid sequence of SEQ ID NO: 17, or the amino acid sequence of SEQ ID NO: 17 with 1, 2, or 3 amino acid modifications.

In some embodiments, the anti-PD1 antibody comprises a VH comprising a VH CDR1 that comprises an amino acid sequence of SEQ ID NO: 12, with 0, 1, 2, or 3 amino acid modifications; a VH CDR2 that comprises an amino acid sequence of SEQ ID NO: 13, with 0, 1, 2, or 3 amino acid modifications; and a VH CDR3 that comprises an amino acid sequence of SEQ ID NO: 14, with 0, 1, 2, or 3 amino acid modifications; and the anti-PD1 antibody comprises a VL that comprises a VL CDR1 that comprises an amino acid sequence of SEQ ID NO: 15, with 0, 1, 2, or 3 amino acid modifications; a VL CDR2 that comprises an amino acid sequence of SEQ ID NO: 16, with 0, 1, 2, or 3 amino acid modifications; and a VL CDR3 that comprises an amino acid sequence of SEQ ID NO: 17, with 0, 1, 2, or 3 amino acid modifications.

In some embodiments, the anti-PD1 antibody comprises a VH comprising a VH CDR1 that comprises the amino acid sequence of SEQ ID NO: 12, or the amino acid sequence of SEQ ID NO: 12 with 1, 2, or 3 amino acid modifications; a VH CDR2 that comprises the amino acid sequence of SEQ ID NO: 13, or the amino acid sequence of SEQ ID NO: 13 with 1, 2, or 3 amino acid modifications; and a VH CDR3 that comprises the amino acid sequence of SEQ ID NO: 14, or the amino acid sequence of SEQ ID NO: 14 with 1, 2, or 3 amino acid modifications; and a VL that comprises a VL CDR1 that comprises an amino acid sequence of SEQ ID NO: 15, or the amino acid sequence of SEQ ID NO: 15 with 1, 2, or 3 amino acid modifications; a VL CDR2 that comprises an amino acid sequence of SEQ ID NO: 16, or the amino acid sequence of SEQ ID NO: 16 with 1, 2, or 3 amino acid modifications; and a VL CDR3 that comprises an amino acid sequence of SEQ ID NO: 17, or the amino acid sequence of SEQ ID NO: 17 with 1, 2, or 3 amino acid modifications.

In some embodiments, the anti-PD1 antibody comprises a VH comprising a VH CDR1 that comprises an amino acid sequence at least 95%, 96%, 97%, 98%, 99%, or 100% identical to the amino acid sequence of SEQ ID NO: 12; a VH CDR2 that comprises an amino acid sequence at least 95%, 96%, 97%, 98%, 99%, or 100% identical to the amino acid sequence of SEQ ID NO: 13; and a VH CDR3 that comprises an amino acid sequence at least 95%, 96%, 97%, 98%, 99%, or 100% identical to the amino acid sequence of SEQ ID NO: 14.

In some embodiments, the anti-PD1 antibody comprises a VL comprising a VL CDR1 that comprises an amino acid sequence at least 95%, 96%, 97%, 98%, 99%, or 100% identical to the amino acid sequence of SEQ ID NO: 15; a VL CDR2 that comprises an amino acid sequence at least 95%, 96%, 97%, 98%, 99%, or 100% identical to the amino acid sequence of SEQ ID NO: 16; and a VL CDR3 that comprises an amino acid sequence at least 95%, 96%, 97%, 98%, 99%, or 100% identical to the amino acid sequence of SEQ ID NO: 17.

In some embodiments, the anti-PD1 antibody comprises a VH comprising a VH CDR1 that comprises an amino acid sequence at least 95%, 96%, 97%, 98%, 99%, or 100% identical to the amino acid sequence of SEQ ID NO: 12; a VH CDR2 that comprises an amino acid sequence at least 95%, 96%, 97%, 98%, 99%, or 100% identical to the amino acid sequence of SEQ ID NO: 13; and a VH CDR3 that comprises an amino acid sequence at least 95%, 96%, 97%, 98%, 99%, or 100% identical to the amino acid sequence of SEQ ID NO: 14; and the anti-PD1 antibody comprises a VL comprising a VL CDR1 that comprises an amino acid sequence at least 95%, 96%, 97%, 98%, 99%, or 100% identical to the amino acid sequence of SEQ ID NO: 15; a VL CDR2 that comprises an amino acid sequence at least 95%, 96%, 97%, 98%, 99%, or 100% identical to the amino acid sequence of SEQ ID NO: 16; and a VL CDR3 that comprises an amino acid sequence at least 95%, 96%, 97%, 98%, 99%, or 100% identical to the amino acid sequence of SEQ ID NO: 17.

In some embodiments, the anti-PD1 antibody comprises a VH at least 90%, 91%, 92%, 93%, 94%, 95%, 96%, 97%, 98%, 99%, or 100% identical to the amino acid sequence of SEQ ID NO: 18. In some embodiments, the anti-PD1 antibody comprises a VL at least 90%, 91%, 92%, 93%, 94%, 95%, 96%, 97%, 98%, 99%, or 100% identical to the amino acid sequence of SEQ ID NO: 19. In some embodiments, the anti-PD1 antibody comprises a VH at least 90%, 91%, 92%, 93%, 94%, 95%, 96%, 97%, 98%, 99%, or 100% identical to the amino acid sequence of SEQ ID NO: 18; and a VL at least 90%, 91%, 92%, 93%, 94%, 95%, 96%, 97%, 98%, 99%, or 100% identical to the amino acid sequence of SEQ ID NO: 19.

In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 21. In some embodiments, the anti-PD1 antibody comprises a light chain that comprises an amino acid sequence at least 90%, 91%, 92%, 93%, 94%, 95%, 96%, 97%, 98%, 99%, or 100% identical to the amino acid sequence of SEQ ID NO: 22. In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 21; and a light chain that comprises an amino acid sequence at least 90%, 91%, 92%, 93%, 94%, 95%, 96%, 97%, 98%, 99%, or 100% identical to the amino acid sequence of SEQ ID NO: 22.

In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 20. In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 20; and a light chain that comprises an amino acid sequence at least 90%, 91%, 92%, 93%, 94%, 95%, 96%, 97%, 98%, 99%, or 100% identical to the amino acid sequence of SEQ ID NO: 22.

### Cemiplimab

In some embodiments, the anti-PD1 antibody is cemiplimab, which is a monoclonal antibody against the PD1 receptor. In some embodiments, the anti-PD1 antibody cross-competes with cemiplimab. In some embodiments, the anti-PD-1 antibody binds to the same epitope as cemiplimab. In some embodiments, the anti-PD-1 antibody has the same CDR regions as cemiplimab.

In some embodiments the anti-PD1 antibody comprises a variable heavy chain (VH) that comprises three complementarity determining regions: VH CDR1, VH CDR2, and VH CDR3. In some embodiments, the anti-PD1 antibody comprises a VH comprising a VH CDR1 that comprises an amino acid sequence of SEQ ID NO: 23 with 0, 1, 2, or 3 amino acid modifications; a VH CDR2 that comprises an amino acid sequence of SEQ ID NO: 24, with 0, 1, 2, or 3 amino acid modifications; and a VH CDR3 that comprises an amino acid sequence of SEQ ID NO: 25, with 0, 1, 2, or 3 amino acid modifications. In some embodiments, the anti-PD1 antibody comprises a VH comprising a VH CDR1 that comprises the amino acid sequence of SEQ ID NO: 23, or the amino acid sequence of SEQ ID NO: 23 with 1, 2, or 3 amino acid modifications; a VH CDR2 that comprises the amino acid sequence of SEQ ID NO: 24, or the amino acid sequence of SEQ ID NO: 24 with 1, 2, or 3 amino acid modifications; and a VH CDR3 that comprises the amino acid sequence of SEQ ID NO: 25, or the amino acid sequence of SEQ ID NO: 25 with 1, 2, or 3 amino acid modifications.

In some embodiments the anti-PD1 antibody comprises a variable light chain (VL) that comprises three complementarity determining regions: VL CDR1, VL CDR2, and VL CDR3. In some embodiments, the anti-PD1 antibody comprises a VL comprising a VL CDR1 that comprises an amino acid sequence of SEQ ID NO: 26, with 0, 1, 2, or 3 amino acid modifications; a VL CDR2 that comprises an amino acid sequence of SEQ ID NO: 27, with 0, 1, 2, or 3 amino acid modifications; and a VL CDR3 that comprises an amino acid sequence of SEQ ID NO: 28, with 0, 1, 2, or 3 amino acid modifications. In some embodiments, the anti-PD1 antibody comprises a VL comprising a VL CDR1 that comprises the amino acid sequence of SEQ ID NO: 26, or the amino acid sequence of SEQ ID NO: 26 with 1, 2, or 3 amino acid modifications; a VL CDR2 that comprises the amino acid sequence of SEQ ID NO: 27, or the amino acid sequence of SEQ ID NO: 27 with 1, 2, or 3 amino acid modifications; and a VL CDR3 that comprises the amino acid sequence of SEQ ID NO: 28, or the amino acid sequence of SEQ ID NO: 28 with 1, 2, or 3 amino acid modifications.

In some embodiments, the anti-PD1 antibody comprises a VH comprising a VH CDR1 that comprises an amino acid sequence of SEQ ID NO: 23, with 0, 1, 2, or 3 amino acid modifications; a VH CDR2 that comprises an amino acid sequence of SEQ ID NO: 24, with 0, 1, 2, or 3 amino acid modifications; and a VH CDR3 that comprises an amino acid sequence of SEQ ID NO: 25, with 0, 1, 2, or 3 amino acid modifications; and a VL comprising a VL CDR1 that comprises an amino acid sequence of SEQ ID NO: 26, with 0, 1, 2, or 3 amino acid modifications; a VL CDR2 that comprises an amino acid sequence of SEQ ID NO: 27, with 0, 1, 2, or 3 amino acid modifications; and a VL CDR3 that comprises an amino acid sequence of SEQ ID NO: 28, with 0, 1, 2, or 3 amino acid modifications.

In some embodiments, the anti-PD1 antibody comprises a VH comprising a VH CDR1 that comprises the amino acid sequence of SEQ ID NO: 23, or the amino acid sequence of SEQ ID NO: 26 with 1, 2, or 3 amino acid modifications; a VH CDR2 that comprises the amino acid sequence of SEQ ID NO: 24, or the amino acid sequence of SEQ ID NO: 24 with 1, 2, or 3 amino acid modifications; and a VH CDR3 that comprises the amino acid sequence of SEQ ID NO: 25, or the amino acid sequence of SEQ ID NO: 25 with 1, 2, or 3 amino acid modifications; and a VL comprising a VL CDR1 that comprises the amino acid sequence of SEQ ID NO: 26, or the amino acid sequence of SEQ ID NO: 26 with 1, 2, or 3 amino acid modifications; a VL CDR2 that comprises the amino acid sequence of SEQ ID NO: 27, or the amino acid sequence of SEQ ID NO: 27 with 1, 2, or 3 amino acid modifications; and a VL CDR3 that comprises the amino acid sequence of SEQ ID NO: 28, or the amino acid sequence of SEQ ID NO: 28 with 1, 2, or 3 amino acid modifications.

In some embodiments, the anti-PD1 antibody comprises a VH comprising a VH CDR1 that comprises an amino acid sequence at least 95%, 96%, 97%, 98%, 99%, or 100% identical to the amino acid sequence of SEQ ID NO: 23; a VH CDR2 that comprises an amino acid sequence at least 95%, 96%, 97%, 98%, 99%, or 100% identical to the amino acid sequence of SEQ ID NO: 24; and a VH CDR3 that comprises an amino acid sequence at least 95%, 96%, 97%, 98%, 99%, or 100% identical to the amino acid sequence of SEQ ID NO: 25.

In some embodiments, the anti-PD1 antibody comprises a VL comprising a VL CDR1 that comprises an amino acid sequence at least 95%, 96%, 97%, 98%, 99%, or 100% identical to the amino acid sequence of SEQ ID NO: 26; a VL CDR2 that comprises an amino acid sequence at least 95%, 96%, 97%, 98%, 99%, or 100% identical to the amino acid sequence of SEQ ID NO: 27; and a VL CDR3 that comprises an amino acid sequence at least 95%, 96%, 97%, 98%, 99%, or 100% identical to the amino acid sequence of SEQ ID NO: 28.

In some embodiments, the anti-PD1 antibody comprises a VH comprising a VH CDR1 that comprises an amino acid sequence at least 95%, 96%, 97%, 98%, 99%, or 100% identical to the amino acid sequence of SEQ ID NO: 23; a VH CDR2 that comprises an amino acid sequence at least 95%, 96%, 97%, 98%, 99%, or 100% identical to the amino acid sequence of SEQ ID NO: 24; and a VH CDR3 that comprises an amino acid sequence at least 95%, 96%, 97%, 98%, 99%, or 100% identical to the amino acid sequence of SEQ ID NO: 25; and the anti-PD1 antibody comprises a VL comprising a VL CDR1 that comprises an amino acid sequence at least 95%, 96%, 97%, 98%, 99%, or 100% identical to the amino acid sequence of SEQ ID NO: 26; a VL CDR2 that comprises an amino acid sequence at least 95%, 96%, 97%, 98%, 99%, or 100% identical to the amino acid sequence of SEQ ID NO: 27; and a VL CDR3 that comprises an amino acid sequence at least 95%, 96%, 97%, 98%, 99%, or 100% identical to the amino acid sequence of SEQ ID NO: 28.

In some embodiments, the anti-PD1 antibody comprises a VH at least 90%, 91%, 92%, 93%, 94%, 95%, 96%, 97%, 98%, 99%, or 100% identical to the amino acid sequence of SEQ ID NO: 29. In some embodiments, the anti-PD1 antibody comprises a VL at least 90%, 91%, 92%, 93%, 94%, 95%, 96%, 97%, 98%, 99%, or 100% identical to the amino acid sequence of SEQ ID NO: 30. In some embodiments, the anti-PD1 antibody comprises a VH at least 90%, 91%, 92%, 93%, 94%, 95%, 96%, 97%, 98%, 99%, or 100% identical to the amino acid sequence of SEQ ID NO: 29; and a VL at least 90%, 91%, 92%, 93%, 94%, 95%, 96%, 97%, 98%, 99%, or 100% identical to the amino acid sequence of SEQ ID NO: 30.

In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 32. In some embodiments, the anti-PD1 antibody comprises a light chain that comprises an amino acid sequence at least 90%, 91%, 92%, 93%, 94%, 95%, 96%, 97%, 98%, 99%, or 100% identical to the amino acid sequence of SEQ ID NO: 33. In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 32; and a light chain that comprises an amino acid sequence at least 90%, 91%, 92%, 93%, 94%, 95%, 96%, 97%, 98%, 99%, or 100% identical to the amino acid sequence of SEQ ID NO: 33.

In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 31. In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 31; and a light chain that comprises an amino acid sequence at least 90%, 91%, 92%, 93%, 94%, 95%, 96%, 97%, 98%, 99%, or 100% identical to the amino acid sequence of SEQ ID NO: 33.

In some embodiments, the anti-PD1 antibody comprises an antibody in Table 1. In some embodiments, the anti-PD1 antibody is an antibody in Table 1.

In some embodiments, the anti-PD1 antibody comprises a VH comprising a VH CDR1 that comprises an amino acid sequence at least 95%, 96%, 97%, 98%, 99%, or 100% identical to the amino acid sequence of a VH CDR1 in Table 1; a VH CDR2 that comprises an amino acid sequence at least 95%, 96%, 97%, 98%, 99%, or 100% identical to the amino acid sequence of a VH CDR2 in Table 1; and a VH CDR3 that comprises an amino acid sequence at least 95%, 96%, 97%, 98%, 99%, or 100% identical to the amino acid sequence of a VH CDR3 in Table 1.

In some embodiments, the anti-PD1 antibody comprises a VL comprising a VL CDR1 that comprises an amino acid sequence at least 95%, 96%, 97%, 98%, 99%, or 100% identical to the amino acid sequence of a VL CDR1 in Table 1; a VL CDR2 that comprises an amino acid sequence at least 95%, 96%, 97%, 98%, 99%, or 100% identical to the amino acid sequence of a VL CDR2 in Table 1; and a VL CDR3 that comprises an amino acid sequence at least 95%, 96%, 97%, 98%, 99%, or 100% identical to the amino acid sequence of a VL CDR3 in Table 1.

In some embodiments, the anti-PD1 antibody comprises a VH comprising a VH CDR1 that comprises an amino acid sequence at least 95%, 96%, 97%, 98%, 99%, or 100% identical to the amino acid sequence of a VH CDR1 in Table 1; a VH CDR2 that comprises an amino acid sequence at least 95%, 96%, 97%, 98%, 99%, or 100% identical to the amino acid sequence of a VH CDR2 in Table 1; a VH CDR3 that comprises an amino acid sequence at least 95%, 96%, 97%, 98%, 99%, or 100% identical to the amino acid sequence of a VH CDR3 in Table 1; a VL comprising a VL CDR1 that comprises an amino acid sequence at least 95%, 96%, 97%, 98%, 99%, or 100% identical to the amino acid sequence of a VL CDR1 in Table 1; a VL CDR2 that comprises an amino acid sequence at least 95%, 96%, 97%, 98%, 99%, or 100% identical to the amino acid sequence of a VL CDR2 in Table 1; and a VL CDR3 that comprises an amino acid sequence at least 95%, 96%, 97%, 98%, 99%, or 100% identical to the amino acid sequence of a VL CDR3 in Table 1.

In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of a heavy chain in Table 1. In some embodiments, the anti-PD1 antibody comprises a light chain that comprises an amino acid sequence at least 90%, 91%, 92%, 93%, 94%, 95%, 96%, 97%, 98%, 99%, or 100% identical to the amino acid sequence of a light chain in Table 1. In some embodiments, the anti-PD1 antibody comprises a heavy chain that comprises an amino acid sequence at least 90%, 91%, 92%, 93%, 94%, 95%, 96%, 97%, 98%, 99%, or 100% identical to the amino acid sequence of a heavy chain in Table 1; and a light chain that comprises an amino acid sequence at least 90%, 91%, 92%, 93%, 94%, 95%, 96%, 97%, 98%, 99%, or 100% identical to the amino acid sequence of a light chain in Table 1.

In some embodiments, the anti-PD1 antibody comprises a VH that comprises an amino acid sequence at least 95%, 96%, 97%, 98%, 99%, or 100% identical to the amino acid sequence of a VH of an antibody in Table 1. In some embodiments, the anti-PD1 antibody comprises a VL that comprises an amino acid sequence at least 95%, 96%, 97%, 98%, 99%, or 100% identical to the amino acid sequence of an antibody in Table 1. In some embodiments, the anti-PD1 antibody comprises a VH that comprises an amino acid sequence at least 95%, 96%, 97%, 98%, 99%, or 100% identical to the amino acid sequence of a VH of an antibody in Table 1; and a VL that comprises an amino acid sequence at least 95%, 96%, 97%, 98%, 99%, or 100% identical to the amino acid sequence of an antibody in Table 1.

TABLE 1
Exemplary Anti-PD1 Antibodies

| Antibody | Region | Amino Acid Sequence | SEQ ID NO |
|---|---|---|---|
| Pembrolizumab | VH CDR1 | NYYMY | 1 |
| (CDRs defined | | | |
| according to | | | |
| Kabat) | | | |
| | VH CDR2 | GINPSNGGTNFNEKFKN | 2 |
| | VH CDR3 | RDYRFDMGEDY | 3 |
| | VL CDR1 | RASKGVSTSGYSYLH | 4 |
| | VL CDR2 | LASYLES | 5 |
| | VL CDR3 | QHSRDLPLT | 6 |
| | VH | QVQLVQSGVEVKKPGASVKVSCKASGYTFTNYYMYWVRQAPGQ | 7 |
| | | GLEWMGGINPSNGGTNFNEKFKNRVTLTTDSSTTTAYMELKSL | |
| | | QFDDTAVYYCARRDYRFDMGFDYWGQGTTVTVSS | |
| | VL | EIVLTQSPATLSLSPGERATLSCRASKGVSTSGYSYLHWYQQK | 8 |
| | | PGQAPRLLIYLASYLESGVPARFSGSGSGTDFTLTISSLEPED | |
| | | FAVYYCQHSRDLPLTFGGGTKVEIK | |
| | HC-A | QVQLVQSGVEVKKPGASVKVSCKASGYTFTNYYMYWVRQAPGQ | 9 |
| | | GLEWMGGINPSNGGTNFNEKFKNRVTLTTDSSTTTAYMELKSL | |
| | | QFDDTAVYYCARRDYRFDMGFDYWGQGTTVTVSSASTKGPSVF | |
| | | PLAPCSRSTSESTAALGCLVKDYFPEPVTVSWNSGALTSGVHT | |
| | | FPAVLQSSGLYSLSSVVTVPSSSLGTKTYTCNVDHKPSNTKVD | |
| | | KRVESKYGPPCPPCPAPEFLGGPSVFLFPPKPKDTLMISRTPE | |
| | | VTCVVVDVSQEDPEVQFNWYVDGVEVHNAKTKPREEQFNSTYR | |
| | | VVSVLTVLHQDWLNGKEYKCKVSNKGLPSSIEKTISKAKGQPR | |
| | | EPQVYTLPPSQEEMTKNQVSLTCLVKGFYPSDIAVEWESNGQP | |
| | | ENNYKTTPPVLDSDGSFFLYSRLTVDKSRWQEGNVFSCSVMHE | |
| | | ALHNHYTQKSLSLSLGK | |
| | HC-B | QVQLVQSGVEVKKPGASVKVSCKASGYTFTNYYMYWVRQAPGQ | 10 |
| | (No C- | GLEWMGGINPSNGGTNFNEKFKNRVTLTTDSSTTTAYMELKSL | |
| | terminal | QFDDTAVYYCARRDYRFDMGFDYWGQGTTVTVSSASTKGPSVF | |
| | Lysine) | PLAPCSRSTSESTAALGCLVKDYFPEPVTVSWNSGALTSGVHT | |
| | | FPAVLQSSGLYSLSSVVTVPSSSLGTKTYTCNVDHKPSNTKVD | |
| | | KRVESKYGPPCPPCPAPEFLGGPSVFLFPPKPKDTLMISRTPE | |
| | | VTCVVVDVSQEDPEVQFNWYVDGVEVHNAKTKPREEQFNSTYR | |
| | | VVSVLTVLHQDWLNGKEYKCKVSNKGLPSSIEKTISKAKGQPR | |
| | | EPQVYTLPPSQEEMTKNQVSLTCLVKGFYPSDIAVEWESNGQP | |
| | | ENNYKTTPPVLDSDGSFFLYSRLTVDKSRWQEGNVFSCSVMHE | |
| | | ALHNHYTQKSLSLSLG | |
| | LC | EIVLTQSPATLSLSPGERATLSCRASKGVSTSGYSYLHWYQQK | 11 |
| | | PGQAPRLLIYLASYLESGVPARFSGSGSGTDFTLTISSLEPED | |
| | | FAVYYCQHSRDLPLTFGGGTKVEIKRTVAAPSVFIFPPSDEQL | |
| | | KSGTASVVCLLNNFYPREAKVQWKVDNALQSGNSQESVTEQDS | |
| | | KDSTYSLSSTLTLSKADYEKHKVYACEVTHQGLSSPVTKSENR | |
| | | GEC | |
| Nivolumab | VH CDR1 | NSGMH | 12 |
| (CDRs defined | | | |
| according to | | | |
| Kabat) | | | |
| | VH CDR2 | VIWYDGSKRYYADSVKG | 13 |
| | VH CDR3 | NDDY | 14 |
| | VL CDR1 | RASQSVSSYLA | 15 |
| | VL CDR2 | DASNRAT | 16 |
| | VL CDR3 | QQSSNWPRT | 17 |
| | VH | QVQLVESGGGVVQPGRSLRLDCKASGITFSNSGMHWVRQAPGK | 18 |
| | | GLEWVAVIWYDGSKRYYADSVKGRFTISRDNSKNTLFLQMNSL | |
| | | RAEDTAVYYCATNDDYWGQGTLVTVSS | |
| | VL | EIVLTQSPATLSLSPGERATLSCRASQSVSSYLAWYQQKPGQA | 19 |
| | | PRLLIYDASNRATGIPARFSGSGSGTDFTLTISSLEPEDFAVY | |
| | | YCQQSSNWPRTFGQGTKVEIK | |
| | HC-A | QVQLVESGGGVVQPGRSLRLDCKASGITFSNSGMHWVRQAPGK | 20 |
| | | GLEWVAVIWYDGSKRYYADSVKGRFTISRDNSKNTLFLQMNSL | |
| | | RAEDTAVYYCATNDDYWGQGTLVTVSSASTKGPSVFPLAPCSR | |
| | | STSESTAALGCLVKDYFPEPVTVSWNSGALTSGVHTFPAVLQS | |
| | | SGLYSLSSVVTVPSSSLGTKTYTCNVDHKPSNTKVDKRVESKY | |
| | | GPPCPPCPAPEFLGGPSVFLFPPKPKDTLMISRTPEVTCVVVD | |
| | | VSQEDPEVQFNWYVDGVEVHNAKTKPREEQFNSTYRVVSVLTV | |
| | | LHQDWLNGKEYKCKVSNKGLPSSIEKTISKAKGQPREPQVYTL | |
| | | PPSQEEMTKNQVSLTCLVKGFYPSDIAVEWESNGQPENNYKTT | |
| | | PPVLDSDGSFFLYSRLTVDKSRWQEGNVFSCSVMHEALHNHYT | |
| | | QKSLSLSLGK | |
| | HC-B | QVQLVESGGGVVQPGRSLRLDCKASGITFSNSGMHWVRQAPGK | 21 |
| | (No C- | GLEWVAVIWYDGSKRYYADSVKGRFTISRDNSKNTLFLQMNSL | |
| | terminal | RAEDTAVYYCATNDDYWGQGTLVTVSSASTKGPSVFPLAPCSR | |
| | Lysine) | STSESTAALGCLVKDYFPEPVTVSWNSGALTSGVHTFPAVLQS | |
| | | SGLYSLSSVVTVPSSSLGTKTYTCNVDHKPSNTKVDKRVESKY | |
| | | GPPCPPCPAPEFLGGPSVFLFPPKPKDTLMISRTPEVTCVVVD | |
| | | VSQEDPEVQFNWYVDGVEVHNAKTKPREEQFNSTYRVVSVLTV | |
| | | LHQDWLNGKEYKCKVSNKGLPSSIEKTISKAKGQPREPQVYTL | |
| | | PPSQEEMTKNQVSLTCLVKGFYPSDIAVEWESNGQPENNYKTT | |
| | | PPVLDSDGSFFLYSRLTVDKSRWQEGNVFSCSVMHEALHNHYT | |
| | | QKSLSLSLG | |
| | LC | EIVLTQSPATLSLSPGERATLSCRASQSVSSYLAWYQQKPGQA | 22 |
| | | PRLLIYDASNRATGIPARFSGSGSGTDFTLTISSLEPEDFAVY | |
| | | YCQQSSNWPRTFGQGTKVEIKRTVAAPSVFIFPPSDEQLKSGT | |
| | | ASVVCLLNNFYPREAKVQWKVDNALQSGNSQESVTEQDSKDST | |
| | | YSLSSTLTLSKADYEKHKVYACEVTHQGLSSPVTKSENRGEC | |
| Cemiplimab | VH CDR1 | NFGMT | 23 |
| (CDRs defined | | | |
| according to | | | |
| Kabat) | | | |
| | VH CDR2 | GISGGGRDTYFADSVKG | 24 |
| | VH CDR3 | WGNIYFDY | 25 |
| | VL CDR1 | RASLSINTELN | 26 |
| | VL CDR2 | AASSLHG | 27 |
| | VL CDR3 | QQSSNTPFT | 28 |
| | VH | EVQLLESGGVLVQPGGSLRLSCAASGFTFSNFGMTWVRQAPGK | 29 |
| | | GLEWVSGISGGGRDTYFADSVKGRFTISRDNSKNTLYLQMNSL | |
| | | KGEDTAVYYCVKWGNIYFDYWGQGTLVT | |
| | VL | DIQMTQSPSSLSASVGDSITITCRASLSINTFLNWYQQKPGKA | 30 |
| | | PNLLIYAASSLHGGVPSRFSGSGSGTDFTLTIRTLQPEDFATY | |
| | | YCQQSSNTPFTFGPGTVVDFR | |
| | HC-A | EVQLLESGGVLVQPGGSLRLSCAASGFTFSNFGMTWVRQAPGK | 31 |
| | | GLEWVSGISGGGRDTYFADSVKGRFTISRDNSKNTLYLQMNSL | |
| | | KGEDTAVYYCVKWGNIYFDYWGQGTLVTVSSASTKGPSVFPLA | |
| | | PCSRSTSESTAALGCLVKDYFPEPVTVSWNSGALTSGVHTFPA | |
| | | VLQSSGLYSLSSVVTVPSSSLGTKTYTCNVDHKPSNTKVDKRV | |
| | | ESKYGPPCPPCPAPEFLGGPSVFLFPPKPKDTLMISRTPEVTC | |
| | | VVVDVSQEDPEVQFNWYVDGVEVHNAKTKPREEQFNSTYRVVS | |
| | | VLTVLHQDWLNGKEYKCKVSNKGLPSSIEKTISKAKGQPREPQ | |
| | | VYTLPPSQEEMTKNQVSLTCLVKGFYPSDIAVEWESNGQPENN | |
| | | YKTTPPVLDSDGSFFLYSRLTVDKSRWQEGNVFSCSVMHEALH | |
| | | NHYTQKSLSLSLGK | |
| | HC-B | EVQLLESGGVLVQPGGSLRLSCAASGFTFSNFGMTWVRQAPGK | 32 |
| | (No C- | GLEWVSGISGGGRDTYFADSVKGRFTISRDNSKNTLYLQMNSL | |
| | terminal | KGEDTAVYYCVKWGNIYFDYWGQGTLVTVSSASTKGPSVFPLA | |
| | Lysine) | PCSRSTSESTAALGCLVKDYFPEPVTVSWNSGALTSGVHTFPA | |
| | | VLQSSGLYSLSSVVTVPSSSLGTKTYTCNVDHKPSNTKVDKRV | |
| | | ESKYGPPCPPCPAPEFLGGPSVFLFPPKPKDTLMISRTPEVTC | |
| | | VVVDVSQEDPEVQFNWYVDGVEVHNAKTKPREEQFNSTYRVVS | |
| | | VLTVLHQDWLNGKEYKCKVSNKGLPSSIEKTISKAKGQPREPQ | |
| | | VYTLPPSQEEMTKNQVSLTCLVKGFYPSDIAVEWESNGQPENN | |
| | | YKTTPPVLDSDGSFFLYSRLTVDKSRWQEGNVFSCSVMHEALH | |
| | | NHYTQKSLSLSLG | |
| | LC | DIQMTQSPSSLSASVGDSITITCRASLSINTFLNWYQQKPGKA | 33 |
| | | PNLLIYAASSLHGGVPSRFSGSGSGTDFTLTIRTLQPEDFATY | |
| | | YCQQSSNTPFTFGPGTVVDFRRTVAAPSVFIFPPSDEQLKSGT | |
| | | ASVVCLLNNFYPREAKVQWKVDNALQSGNSQESVTEQDSKDST | |
| | | YSLSSTLTLSKADYEKHKVYACEVTHQGLSSPVTKSENRGEC | |

### Methods of Making Anti-PD1 Antibodies

Anti-PD1 antibodies described herein can be made by any conventional technique known in the art, for example, recombinant techniques or chemical synthesis (e.g., solid phase peptide synthesis). In one embodiments, the anti-PD1 antibody is made through recombinant expression in a cell. Briefly, the anti-PD1 antibody can be made by synthesizing the DNA encoding the anti-PD1 antibody and cloning the DNA into any suitable expression vector. Numerous cloning vectors are known to those of skill in the art, and the selection of an appropriate cloning vector is a matter of choice. The gene can be placed under the control of a promoter, ribosome binding site (for bacterial expression) and, optionally, an operator, so that the DNA sequence encoding the fusion protein is transcribed into RNA in the host cell transformed by a vector containing this expression construction. The coding sequence may or may not contain a signal peptide or leader sequence. Heterologous leader sequences can be added to the coding sequence that causes the secretion of the expressed polypeptide from the host organism. Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences. The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

The expression vector may then used to transform an appropriate host cell. A number of mammalian cell lines are known in the art and include immortalized cell lines available from the American Type Culture Collection (ATCC), such as, but not limited to, Chinese hamster ovary (CHO) cells, CHO-suspension cells (CHO-S), HeLa cells, HEK293, baby hamster kidney (BHK) cells, monkey kidney cells (COS), VERO, HepG2, MadinDarby bovine kidney (MDBK) cells, NOS, U2OS, A549, HT1080, CAD, P19, NIH3T3, L929, N2a, MCF-7, Y79, SO-Rb50, DUKX-X11, and J558L. In some embodiments, the anti-PD1 antibody is produced in CHO or CHO-S cells.

Depending on the expression system and host selected, the anti-PD1 antibody is produced by growing host cells transformed by an expression vector described above under conditions whereby the anti-PD1 antibody is expressed. The anti-PD1 antibody is then isolated from the host cells and purified. If the expression system secretes the anti-PD1 antibody into growth media, the anti-PD1 antibody can be purified directly from the media. If the anti-PD1 antibody is not secreted, it is isolated from cell lysates. The selection of the appropriate growth conditions and recovery methods are within the skill of the art. Once purified, the amino acid sequences of the anti-PD1 antibody can be determined, i.e., by repetitive cycles of Edman degradation, followed by amino acid analysis by HPLC. Other methods of amino acid sequencing are also known in the art. Once purified, the functionality of the anti-PD1 antibody can be assessed by any conventional method known in the art, e.g., ELISA.

### Fusion Proteins

In certain aspects, provided herein are methods of treating cancer comprising administering to a subject having cancer an antibody, or functional fragment or functional variant thereof, that specifically binds programmed death protein 1 (PD1) e.g., described herein, in combination with a fusion protein that comprises a targeting moiety and an immunomodulatory moiety, wherein: i) said targeting moiety specifically binds epidermal growth factor receptor (EGFR); and (ii) said immunomodulatory moiety comprises an amino acid sequence of the extracellular domain of transforming growth factor-beta receptor II (TGFβRII).

### EGFR Targeting Moieties

In some embodiments, the EGFR targeting moiety comprises an antibody, or a functional fragment or functional variant thereof. In some embodiments, the antibody is a full-length antibody, a single chain variable fragment (scFv), a scFv2, a scFv-Fc, a Fab, a Fab′, a F(ab′)2, a F(v), a single domain antibody, a single chain antibody, or a VHH. In some embodiments, the EGFR targeting moiety binds EGFR and inhibits downstream signaling through the bound EGF receptor.

In some embodiments, the anti-EGFR antibody is selected from the group consisting of cetuximab and panitumumab. In some embodiments, the anti-EGFR antibody is a functional fragment of cetuximab and panitumumab. In some embodiments, the anti-EGFR antibody is a functional variant of cetuximab and panitumumab.

### Cetuximab

In some embodiments, the anti-EGFR antibody is cetuximab. In some embodiments, the anti-EGFR antibody cross-competes with cetuximab. In some embodiments, the anti-EGFR antibody binds to the same epitope as cetuximab. In some embodiments, the anti-EGFR antibody has the same CDRs as cetuximab.

In some embodiments the anti-EGFR antibody comprises a variable heavy chain (VH) that comprises three complementarity determining regions: VH CDR1, VH CDR2, and VH CDR3. In some embodiments, the anti-EGFR antibody comprises a VH comprising a VH CDR1 that comprises an amino acid sequence of SEQ ID NO: 34, with 0, 1, 2, or 3 amino acid modifications; a VH CDR2 that comprises an amino acid sequence of SEQ ID NO: 35, with 0, 1, 2, or 3 amino acid modifications; and a VH CDR3 that comprises an amino acid sequence of SEQ ID NO: 36, with 0, 1, 2, or 3 amino acid modifications. In some embodiments, the anti-EGFR antibody comprises a VH comprising a VH CDR1 that comprises the amino acid sequence of SEQ ID NO: 34, or the amino acid sequence of SEQ ID NO: 34 with 1, 2, or 3 amino acid modifications; a VH CDR2 that comprises the amino acid sequence of SEQ ID NO: 35, or the amino acid sequence of SEQ ID NO: 35 with 1, 2, or 3 amino acid modifications; and a VH CDR3 that comprises the amino acid sequence of SEQ ID NO: 36, or the amino acid sequence of SEQ ID NO: 35 with 1, 2, or 3 amino acid modifications.

In some embodiments the anti-EGFR antibody comprises a variable light chain (VL) that comprises three complementarity determining regions: VL CDR1, VL CDR2, and VL CDR3. In some embodiments, the anti-EGFR antibody comprises a VL comprising a VL CDR1 that comprises an amino acid sequence of SEQ ID NO: 37, with 0, 1, 2, or 3 amino acid modifications; a VL CDR2 that comprises an amino acid sequence of SEQ ID NO: 38, with 0, 1, 2, or 3 amino acid modifications; and a VL CDR3 that comprises an amino acid sequence of SEQ ID NO: 39, with 0, 1, 2, or 3 amino acid modifications. In some embodiments, the anti-EGFR antibody comprises a VL comprising a VL CDR1 that comprises the amino acid sequence of SEQ ID NO: 37, or the amino acid sequence of SEQ ID NO: 37 with 1, 2, or 3 amino acid modifications; a VL CDR2 that comprises the amino acid sequence of SEQ ID NO: 38, or the amino acid sequence of SEQ ID NO: 38 with 1, 2, or 3 amino acid modifications; and a VL CDR3 that comprises the amino acid sequence of SEQ ID NO: 39, or the amino acid sequence of SEQ ID NO: 39 with 1, 2, or 3 amino acid modifications.

In some embodiments, the anti-EGFR antibody comprises a VH comprising a VH CDR1 that comprises an amino acid sequence of SEQ ID NO: 34, with 0, 1, 2, or 3 amino acid modifications; a VH CDR2 that comprises an amino acid sequence of SEQ ID NO: 35, with 0, 1, 2, or 3 amino acid modifications; and a VH CDR3 that comprises an amino acid sequence of SEQ ID NO: 36, with 0, 1, 2, or 3 amino acid modifications; and a VL comprising a VL CDR1 that comprises an amino acid sequence of SEQ ID NO: 37, with 0, 1, 2, or 3 amino acid modifications; a VL CDR2 that comprises an amino acid sequence of SEQ ID NO: 38, with 0, 1, 2, or 3 amino acid modifications; and a VL CDR3 that comprises an amino acid sequence of SEQ ID NO: 39, with 0, 1, 2, or 3 amino acid modifications.

In some embodiments, the anti-EGFR antibody comprises a VH comprising a VH CDR1 that comprises the amino acid sequence of SEQ ID NO: 34, or the amino acid sequence of SEQ ID NO: 34 with 1, 2, or 3 amino acid modifications; a VH CDR2 that comprises the amino acid sequence of SEQ ID NO: 35, or the amino acid sequence of SEQ ID NO: 35 with 1, 2, or 3 amino acid modifications; and a VH CDR3 that comprises the amino acid sequence of SEQ ID NO: 36, or the amino acid sequence of SEQ ID NO: 36 with 1, 2, or 3 amino acid modifications; and a VL comprising a VL CDR1 that comprises the amino acid sequence of SEQ ID NO: 37, or the amino acid sequence of SEQ ID NO: 37 with 1, 2, or 3 amino acid modifications; a VL CDR2 that comprises the amino acid sequence of SEQ ID NO: 38, or the amino acid sequence of SEQ ID NO: 38 with 1, 2, or 3 amino acid modifications; and a VL CDR3 that comprises the amino acid sequence of SEQ ID NO: 39, or the amino acid sequence of SEQ ID NO: 39 with 1, 2, or 3 amino acid modifications.

In some embodiments, the anti-EGFR antibody comprises a VH comprising a VH CDR1 that comprises an amino acid sequence at least 95%, 96%, 97%, 98%, 99%, or 100% identical to the amino acid sequence of SEQ ID NO: 34; a VH CDR2 that comprises an amino acid sequence at least 95%, 96%, 97%, 98%, 99%, or 100% identical to the amino acid sequence of SEQ ID NO: 35; and a VH CDR3 that comprises an amino acid sequence at least 95%, 96%, 97%, 98%, 99%, or 100% identical to the amino acid sequence of SEQ ID NO: 36.

In some embodiments, the anti-EGFR antibody comprises a VL comprising a VL CDR1 that comprises an amino acid sequence at least 95%, 96%, 97%, 98%, 99%, or 100% identical to the amino acid sequence of SEQ ID NO: 37; a VL CDR2 that comprises an amino acid sequence at least 95%, 96%, 97%, 98%, 99%, or 100% identical to the amino acid sequence of SEQ ID NO: 38; and a VL CDR3 that comprises an amino acid sequence at least 95%, 96%, 97%, 98%, 99%, or 100% identical to the amino acid sequence of SEQ ID NO: 39.

In some embodiments, the anti-EGFR antibody comprises a VH comprising a VH CDR1 that comprises an amino acid sequence at least 95%, 96%, 97%, 98%, 99%, or 100% identical to the amino acid sequence of SEQ ID NO: 34; a VH CDR2 that comprises an amino acid sequence at least 95%, 96%, 97%, 98%, 99%, or 100% identical to the amino acid sequence of SEQ ID NO: 35; and a VH CDR3 that comprises an amino acid sequence at least 95%, 96%, 97%, 98%, 99%, or 100% identical to the amino acid sequence of SEQ ID NO: 36; and the anti-EGFR antibody comprises a VL comprising a VL CDR1 that comprises an amino acid sequence at least 95%, 96%, 97%, 98%, 99%, or 100% identical to the amino acid sequence of SEQ ID NO: 37; a VL CDR2 that comprises an amino acid sequence at least 95%, 96%, 97%, 98%, 99%, or 100% identical to the amino acid sequence of SEQ ID NO: 38; and a VL CDR3 that comprises an amino acid sequence at least 95%, 96%, 97%, 98%, 99%, or 100% identical to the amino acid sequence of SEQ ID NO: 39.

In some embodiments, the anti-EGFR antibody comprises a VH at least 90%, 91%, 92%, 93%, 94%, 95%, 96%, 97%, 98%, 99%, or 100% identical to the amino acid sequence of SEQ ID NO: 40. In some embodiments, the anti-EGFR antibody comprises a VL at least 90%, 91%, 92%, 93%, 94%, 95%, 96%, 97%, 98%, 99%, or 100% identical to the amino acid sequence of SEQ ID NO: 41. In some embodiments, the anti-EGFR antibody comprises a VH at least 90%, 91%, 92%, 93%, 94%, 95%, 96%, 97%, 98%, 99%, or 100% identical to the amino acid sequence of SEQ ID NO: 40; and a VL at least 90%, 91%, 92%, 93%, 94%, 95%, 96%, 97%, 98%, 99%, or 100% identical to the amino acid sequence of SEQ ID NO: 41.

In some embodiments, the anti-EGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 43. In some embodiments, the anti-EGFR antibody comprises a light chain that comprises an amino acid sequence at least 90%, 91%, 92%, 93%, 94%, 95%, 96%, 97%, 98%, 99%, or 100% identical to the amino acid sequence of SEQ ID NO: 44. In some embodiments, the anti-EGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 43; and a light chain that comprises an amino acid sequence at least 90%, 91%, 92%, 93%, 94%, 95%, 96%, 97%, 98%, 99%, or 100% identical to the amino acid sequence of SEQ ID NO: 44.

In some embodiments, the anti-EGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 42. In some embodiments, the anti-EGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 42; and a light chain that comprises an amino acid sequence at least 90%, 91%, 92%, 93%, 94%, 95%, 96%, 97%, 98%, 99%, or 100% identical to the amino acid sequence of SEQ ID NO: 44.

### Panitumumab

In some embodiments, the anti-EGFR antibody is panitumumab. In some embodiments, the anti-EGFR antibody cross-competes with panitumumab. In some embodiments, the anti-EGFR antibody binds to the same epitope as panitumumab. In some embodiments, the anti-EGFR antibody has the same CDRs as panitumumab.

In some embodiments the anti-EGFR antibody comprises a variable heavy chain (VH) that comprises three complementarity determining regions: VH CDR1, VH CDR2, and VH CDR3. In some embodiments, the anti-EGFR antibody comprises a VH comprising a VH CDR1 that comprises an amino acid sequence of SEQ ID NO: 45, with 0, 1, 2, or 3 amino acid modifications; a VH CDR2 that comprises an amino acid sequence of SEQ ID NO: 46, with 0, 1, 2, or 3 amino acid modifications; and a VH CDR3 that comprises an amino acid sequence of SEQ ID NO: 47, with 0, 1, 2, or 3 amino acid modifications. In some embodiments, the anti-EGFR antibody comprises a VH comprising a VH CDR1 that comprises the amino acid sequence of SEQ ID NO: 45, or the amino acid sequence of SEQ ID NO: 45 with 1, 2, or 3 amino acid modifications; a VH CDR2 that comprises the amino acid sequence of SEQ ID NO: 46, or the amino acid sequence of SEQ ID NO: 46 with 1, 2, or 3 amino acid modifications; and a VH CDR3 that comprises the amino acid sequence of SEQ ID NO: 47, or the amino acid sequence of SEQ ID NO: 47 with 1, 2, or 3 amino acid modifications.

In some embodiments the anti-EGFR antibody comprises a variable light chain (VL) that comprises three complementarity determining regions: VL CDR1, VL CDR2, and VL CDR3. In some embodiments, the anti-EGFR antibody comprises a VL comprising a VL CDR1 that comprises an amino acid sequence of SEQ ID NO: 48, with 0, 1, 2, or 3 amino acid modifications; a VL CDR2 that comprises an amino acid sequence of SEQ ID NO: 49, with 0, 1, 2, or 3 amino acid modifications; and a VL CDR3 that comprises an amino acid sequence of SEQ ID NO: 50, with 0, 1, 2, or 3 amino acid modifications. In some embodiments, the anti-EGFR antibody comprises a VL comprising a VL CDR1 that comprises the amino acid sequence of SEQ ID NO: 48, or the amino acid sequence of SEQ ID NO: 48 with 1, 2, or 3 amino acid modifications; a VL CDR2 that comprises the amino acid sequence of SEQ ID NO: 49, or the amino acid sequence of SEQ ID NO: 49 with 1, 2, or 3 amino acid modifications; and a VL CDR3 that comprises the amino acid sequence of SEQ ID NO: 50, or the amino acid sequence of SEQ ID NO: 50 with 1, 2, or 3 amino acid modifications.

In some embodiments, the anti-EGFR antibody comprises a VH comprising a VH CDR1 that comprises an amino acid sequence of SEQ ID NO: 45, with 0, 1, 2, or 3 amino acid modifications; a VH CDR2 that comprises an amino acid sequence of SEQ ID NO: 46, with 0, 1, 2, or 3 amino acid modifications; and a VH CDR3 that comprises an amino acid sequence of SEQ ID NO: 47, with 0, 1, 2, or 3 amino acid modifications; and a VL comprising a VL CDR1 that comprises an amino acid sequence of SEQ ID NO: 48, with 0, 1, 2, or 3 amino acid modifications; a VL CDR2 that comprises an amino acid sequence of SEQ ID NO: 49, with 0, 1, 2, or 3 amino acid modifications; and a VL CDR3 that comprises an amino acid sequence of SEQ ID NO: 50, with 0, 1, 2, or 3 amino acid modifications.

In some embodiments, the anti-EGFR antibody comprises a VH comprising a VH CDR1 that comprises the amino acid sequence of SEQ ID NO: 45, or the amino acid sequence of SEQ ID NO: 45 with 1, 2, or 3 amino acid modifications; a VH CDR2 that comprises the amino acid sequence of SEQ ID NO: 46, or the amino acid sequence of SEQ ID NO: 46 with 1, 2, or 3 amino acid modifications; and a VH CDR3 that comprises the amino acid sequence of SEQ ID NO: 47, or the amino acid sequence of SEQ ID NO: 47 with 1, 2, or 3 amino acid modifications; and a VL comprising a VL CDR1 that comprises the amino acid sequence of SEQ ID NO: 48, or the amino acid sequence of SEQ ID NO: 48 with 1, 2, or 3 amino acid modifications; a VL CDR2 that comprises the amino acid sequence of SEQ ID NO: 49, or the amino acid sequence of SEQ ID NO: 49 with 1, 2, or 3 amino acid modifications; and a VL CDR3 that comprises an amino acid sequence of SEQ ID NO: 50, or the amino acid sequence of SEQ ID NO: 50 with 1, 2, or 3 amino acid modifications.

In some embodiments, the anti-EGFR antibody comprises a VH comprising a VH CDR1 that comprises an amino acid sequence at least 95%, 96%, 97%, 98%, 99%, or 100% identical to the amino acid sequence of SEQ ID NO: 45; a VH CDR2 that comprises an amino acid sequence at least 95%, 96%, 97%, 98%, 99%, or 100% identical to the amino acid sequence of SEQ ID NO: 46; and a VH CDR3 that comprises an amino acid sequence at least 95%, 96%, 97%, 98%, 99%, or 100% identical to the amino acid sequence of SEQ ID NO: 47.

In some embodiments, the anti-EGFR antibody comprises a VL comprising a VL CDR1 that comprises an amino acid sequence at least 95%, 96%, 97%, 98%, 99%, or 100% identical to the amino acid sequence of SEQ ID NO: 48; a VL CDR2 that comprises an amino acid sequence at least 95%, 96%, 97%, 98%, 99%, or 100% identical to the amino acid sequence of SEQ ID NO: 49; and a VL CDR3 that comprises an amino acid sequence at least 95%, 96%, 97%, 98%, 99%, or 100% identical to the amino acid sequence of SEQ ID NO: 50.

In some embodiments, the anti-EGFR antibody comprises a VH comprising a VH CDR1 that comprises an amino acid sequence at least 95%, 96%, 97%, 98%, 99%, or 100% identical to the amino acid sequence of SEQ ID NO: 45; a VH CDR2 that comprises an amino acid sequence at least 95%, 96%, 97%, 98%, 99%, or 100% identical to the amino acid sequence of SEQ ID NO: 46; and a VH CDR3 that comprises an amino acid sequence at least 95%, 96%, 97%, 98%, 99%, or 100% identical to the amino acid sequence of SEQ ID NO: 47; and the anti-EGFR antibody comprises a VL comprising a VL CDR1 that comprises an amino acid sequence at least 95%, 96%, 97%, 98%, 99%, or 100% identical to the amino acid sequence of SEQ ID NO: 48; a VL CDR2 that comprises an amino acid sequence at least 95%, 96%, 97%, 98%, 99%, or 100% identical to the amino acid sequence of SEQ ID NO: 49; and a VL CDR3 that comprises an amino acid sequence at least 95%, 96%, 97%, 98%, 99%, or 100% identical to the amino acid sequence of SEQ ID NO: 50.

In some embodiments, the anti-EGFR antibody comprises a VH at least 90%, 91%, 92%, 93%, 94%, 95%, 96%, 97%, 98%, 99%, or 100% identical to the amino acid sequence of SEQ ID NO: 51. In some embodiments, the anti-EGFR antibody comprises a VL at least 90%, 91%, 92%, 93%, 94%, 95%, 96%, 97%, 98%, 99%, or 100% identical to the amino acid sequence of SEQ ID NO: 52. In some embodiments, the anti-EGFR antibody comprises a VH at least 90%, 91%, 92%, 93%, 94%, 95%, 96%, 97%, 98%, 99%, or 100% identical to the amino acid sequence of SEQ ID NO: 51; and a VL at least 90%, 91%, 92%, 93%, 94%, 95%, 96%, 97%, 98%, 99%, or 100% identical to the amino acid sequence of SEQ ID NO: 52.

In some embodiments, the anti-EGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 54. In some embodiments, the anti-EGFR antibody comprises a light chain that comprises an amino acid sequence at least 90%, 91%, 92%, 93%, 94%, 95%, 96%, 97%, 98%, 99%, or 100% identical to the amino acid sequence of SEQ ID NO: 55. In some embodiments, the anti-EGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 54; and a light chain that comprises an amino acid sequence at least 90%, 91%, 92%, 93%, 94%, 95%, 96%, 97%, 98%, 99%, or 100% identical to the amino acid sequence of SEQ ID NO: 55.

In some embodiments, the anti-EGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 53. In some embodiments, the anti-EGFR antibody comprises a heavy chain that comprises an amino acid sequence at least 90%, 91%, 92%, 93%, 94%, 95%, 96%, 97%, 98%, 99%, or 100% identical to the amino acid sequence of SEQ ID NO: 53; and a light chain that comprises an amino acid sequence at least 90%, 91%, 92%, 93%, 94%, 95%, 96%, 97%, 98%, 99%, or 100% identical to the amino acid sequence of SEQ ID NO: 55.

In some embodiments, the anti-EGFR antibody comprises an antibody in Table 2. In some embodiments, the anti-EGFR antibody is an antibody in Table 2.

In some embodiments, the anti-EGFR antibody comprises a VH comprising a VH CDR1 that comprises an amino acid sequence at least 95%, 96%, 97%, 98%, 99%, or 100% identical to the amino acid sequence of a VH CDR1 in Table 2; a VH CDR2 that comprises an amino acid sequence at least 95%, 96%, 97%, 98%, 99%, or 100% identical to the amino acid sequence of a VH CDR2 in Table 2; and a VH CDR3 that comprises an amino acid sequence at least 95%, 96%, 97%, 98%, 99%, or 100% identical to the amino acid sequence of a VH CDR3 in Table 2.

In some embodiments, the anti-EGFR antibody comprises a VL comprising a VL CDR1 that comprises an amino acid sequence at least 95%, 96%, 97%, 98%, 99%, or 100% identical to the amino acid sequence of a VL CDR1 in Table 2; a VL CDR2 that comprises an amino acid sequence at least 95%, 96%, 97%, 98%, 99%, or 100% identical to the amino acid sequence of a VL CDR2 in Table 2; and a VL CDR3 that comprises an amino acid sequence at least 95%, 96%, 97%, 98%, 99%, or 100% identical to the amino acid sequence of a VL CDR3 in Table 2.

In some embodiments, the anti-EGFR antibody comprises a VH comprising a VH CDR1 that comprises an amino acid sequence at least 95%, 96%, 97%, 98%, 99%, or 100% identical to the amino acid sequence of a VH CDR1 in Table 2; a VH CDR2 that comprises an amino acid sequence at least 95%, 96%, 97%, 98%, 99%, or 100% identical to the amino acid sequence of a VH CDR2 in Table 2; a VH CDR3 that comprises an amino acid sequence at least 95%, 96%, 97%, 98%, 99%, or 100% identical to the amino acid sequence of a VH CDR3 in Table 2; a VL comprising a VL CDR1 that comprises an amino acid sequence at least 95%, 96%, 97%, 98%, 99%, or 100% identical to the amino acid sequence of a VL CDR1 in Table 2; a VL CDR2 that comprises an amino acid sequence at least 95%, 96%, 97%, 98%, 99%, or 100% identical to the amino acid sequence of a VL CDR2 in Table 2; and a VL CDR3 that comprises an amino acid sequence at least 95%, 96%, 97%, 98%, 99%, or 100% identical to the amino acid sequence of a VL CDR3 in Table 2.

In some embodiments, the anti-EGFR antibody comprises a heavy chain that comprises an amino acid sequence at least 90%, 91%, 92%, 93%, 94%, 95%, 96%, 97%, 98%, 99%, or 100% identical to the amino acid sequence of a heavy chain in Table 2. In some embodiments, the anti-EGFR antibody comprises a light chain that comprises an amino acid sequence at least 90%, 91%, 92%, 93%, 94%, 95%, 96%, 97%, 98%, 99%, or 100% identical to the amino acid sequence of a light chain in Table 2. In some embodiments, the anti-EGFR antibody comprises a heavy chain that comprises an amino acid sequence at least 90%, 91%, 92%, 93%, 94%, 95%, 96%, 97%, 98%, 99%, or 100% identical to the amino acid sequence of a heavy chain in Table 2; and a light chain that comprises an amino acid sequence at least 90%, 91%, 92%, 93%, 94%, 95%, 96%, 97%, 98%, 99%, or 100% identical to the amino acid sequence of a light chain in Table 2.

In some embodiments, the anti-EGFR antibody comprises a VH that comprises an amino acid sequence at least 95%, 96%, 97%, 98%, 99%, or 100% identical to the amino acid sequence of a VH of an antibody in Table 2. In some embodiments, the anti-EGFR antibody comprises a VL that comprises an amino acid sequence at least 95%, 96%, 97%, 98%, 99%, or 100% identical to the amino acid sequence of an antibody in Table 2. In some embodiments, the anti-EGFR antibody comprises a VH that comprises an amino acid sequence at least 95%, 96%, 97%, 98%, 99%, or 100% identical to the amino acid sequence of a VH of an antibody in Table 2; and a VL that comprises an amino acid sequence at least 95%, 96%, 97%, 98%, 99%, or 100% identical to the amino acid sequence of an antibody in Table 2.

TABLE 2
Exemplary Anti-EGFR Antibodies

| | | | SEQ ID |
|---|---|---|---|
| Antibody | Region | Amino Acid Sequence | NO |
| Cetuximab | VH CDR1 | NYGVH | 34 |
| (CDRs defined | | | |
| according | | | |
| to | | | |
| Kabat) | | | |
| | VH CDR2 | VIWSGGNTDYNTPFTS | 35 |
| | VH CDR3 | ALTYYDYEFAY | 36 |
| | VL CDR1 | RASQSIGTNIH | 37 |
| | VL CDR2 | YASESIS | 38 |
| | VL CDR3 | QQNNNWPTT | 39 |
| | VH | QVQLKQSGPGLVQPSQSLSITCTVSGFSLTNYGVHWVRQSP | 40 |
| | | GKGLEWLGVIWSGGNTDYNTPFTSRLSINKDNSKSQVFFKM | |
| | | NSLQSNDTAIYYCARALTYYDYEFAYWGQGTLVTVSA | |
| | VL | DILLTQSPVILSVSPGERVSFSCRASQSIGTNIHWYQQRTN | 41 |
| | | GSPRLLIKYASESISGIPSRFSGSGSGTDFTLSINSVESED | |
| | | IADYYCQQNNNWPTTFGAGTKLELK | |
| | HC-A | QVQLKQSGPGLVQPSQSLSITCTVSGFSLTNYGVHWVRQSP | 42 |
| | VH | GKGLEWLGVIWSGGNTDYNTPFTSRLSINKDNSKSQVFFKM | |
| | Underlined | NSLQSNDTAIYYCARALTYYDYEFAYWGQGTLVTVSAASTK | |
| | CDRs | GPSVFPLAPSSKSTSGGTAALGCLVKDYFPEPVTVSWNSGA | |
| | Underlined | LTSGVHTFPAVLQSSGLYSLSSVVTVPSSSLGTQTYICNVN | |
| | & Bold | HKPSNTKVDKRVEPKSCDKTHTCPPCPAPELLGGPSVFLFP | |
| | | PKPKDTLMISRTPEVTCVVVDVSHEDPEVKFNWYVDGVEVH | |
| | | NAKTKPREEQYNSTYRVVSVLTVLHQDWLNGKEYKCKVSNK | |
| | | ALPAPIEKTISKAKGQPREPQVYTLPPSRDELTKNQVSLTC | |
| | | LVKGFYPSDIAVEWESNGQPENNYKTTPPVLDSDGSFFLYS | |
| | | KLTVDKSRWQQGNVFSCSVMHEALHNHYTQKSLSLSPGK | |
| | HC-B- | QVQLKQSGPGLVQPSQSLSITCTVSGFSLTNYGVHWVRQSP | 43 |
| | Modified | GKGLEWLGVIWSGGNTDYNTPFTSRLSINKDNSKSQVFFKM | |
| | Cetuximab | NSLQSNDTAIYYCARALTYYDYEFAYWGQGTLVTVSAASTK | |
| | (No C-terminal | GPSVFPLAPSSKSTSGGTAALGCLVKDYFPEPVTVSWNSGA | |
| | Lysine) | LTSGVHTFPAVLQSSGLYSLSSVVTVPSSSLGTQTYICNVN | |
| | VH | HKPSNTKVDKRVEPKSCDKTHTCPPCPAPELLGGPSVFLFP | |
| | Underlined | PKPKDTLMISRTPEVTCVVVDVSHEDPEVKFNWYVDGVEVH | |
| | CDRs | NAKTKPREEQYNSTYRVVSVLTVLHQDWLNGKEYKCKVSNK | |
| | Underlined | ALPAPIEKTISKAKGQPREPQVYTLPPSRDELTKNQVSLTC | |
| | & Bold | LVKGFYPSDIAVEWESNGQPENNYKTTPPVLDSDGSFFLYS | |
| | | KLTVDKSRWQQGNVFSCSVMHEALHNHYTQKSLSLSPG | |
| | LC | DILLTQSPVILSVSPGERVSFSCRASQSIGTNIHWYQQRTN | 44 |
| | VL | GSPRLLIKYASESISGIPSRFSGSGSGTDFTLSINSVESED | |
| | Underlined | IADYYCQQNNNWPTTFGAGTKLELKRTVAAPSVFIFPPSDE | |
| | CDRs | QLKSGTASVVCLLNNFYPREAKVQWKVDNALQSGNSQESVT | |
| | Underlined | EQDSKDSTYSLSSTLTLSKADYEKHKVYACEVTHQGLSSPV | |
| | & Bold | TKSENRGEC | |
| Panitumumab | VH CDR1 | SGDYYWT | 45 |
| (CDRs defined | | | |
| according to | | | |
| Kabat) | | | |
| | VH CDR2 | HIYYSGNTNYNPSLKS | 46 |
| | VH CDR3 | DRVTGAFDI | 47 |
| | VL CDR1 | QASQDISNYLN | 48 |
| | VL CDR2 | DASNLET | 49 |
| | VL CDR3 | QHFDHLPLA | 50 |
| | VH | QVQLQESGPGLVKPSETLSLTCTVSGGSVSSGDYYWTWIRQ | 51 |
| | | SPGKGLEWIGHIYYSGNTNYNPSLKSRLTISIDTSKTQFSL | |
| | | KLSSVTAADTAIYYCVRDRVTGAFDIWGQGTMVTVSS | |
| | VL | DIQMTQSPSSLSASVGDRVTITCQASQDISNYLNWYQQKPG | 52 |
| | | KAPKLLIYDASNLETGVPSRFSGSGSGTDFTFTISSLQPED | |
| | | IATYFCQHFDHLPLAFGGGTKVEIK | |
| | HC-A | QVQLQESGPGLVKPSETLSLTCTVSGGSVSSGDYYWTWIRQ | 53 |
| | VH | SPGKGLEWIGHIYYSGNTNYNPSLKSRLTISIDTSKTQFSL | |
| | Underlined | KLSSVTAADTAIYYCVRDRVTGAFDIWGQGTMVTVSSASTK | |
| | CDRs | GPSVFPLAPCSRSTSESTAALGCLVKDYFPEPVTVSWNSGA | |
| | Underlined | LTSGVHTFPAVLQSSGLYSLSSVVTVPSSNFGTQTYTCNVD | |
| | & Bold | HKPSNTKVDKTVERKCCVECPPCPAPPVAGPSVFLFPPKPK | |
| | | DTLMISRTPEVTCVVVDVSHEDPEVQFNWYVDGVEVHNAKT | |
| | | KPREEQFNSTFRVVSVLTVVHQDWLNGKEYKCKVSNKGLPA | |
| | | PIEKTISKTKGQPREPQVYTLPPSREEMTKNQVSLTCLVKG | |
| | | FYPSDIAVEWESNGQPENNYKTTPPMLDSDGSFFLYSKLTV | |
| | | DKSRWQQGNVFSCSVMHEALHNHYTQKSLSLSPGK | |
| | HC-B- | QVQLQESGPGLVKPSETLSLTCTVSGGSVSSGDYYWTWIRQ | 54 |
| | Modified | SPGKGLEWIGHIYYSGNTNYNPSLKSRLTISIDTSKTQFSL | |
| | Panitumumab | KLSSVTAADTAIYYCVRDRVTGAFDIWGQGTMVTVSSASTK | |
| | (No C-terminal | GPSVFPLAPCSRSTSESTAALGCLVKDYFPEPVTVSWNSGA | |
| | Lysine) | LTSGVHTFPAVLQSSGLYSLSSVVTVPSSNFGTQTYTCNVD | |
| | VH | HKPSNTKVDKTVERKCCVECPPCPAPPVAGPSVFLFPPKPK | |
| | Underlined | DTLMISRTPEVTCVVVDVSHEDPEVQFNWYVDGVEVHNAKT | |
| | CDRs | KPREEQFNSTFRVVSVLTVVHQDWLNGKEYKCKVSNKGLPA | |
| | Underlined | PIEKTISKTKGQPREPQVYTLPPSREEMTKNQVSLTCLVKG | |
| | & Bold | FYPSDIAVEWESNGQPENNYKTTPPMLDSDGSFFLYSKLTV | |
| | | DKSRWQQGNVFSCSVMHEALHNHYTQKSLSLSPG | |
| | LC | DIQMTQSPSSLSASVGDRVTITCQASQDISNYLNWYQQKPG | 55 |
| | VL | KAPKLLIYDASNLETGVPSRFSGSGSGTDFTFTISSLQPED | |
| | Underlined | IATYFCQHFDHLPLAFGGGTKVEIKRTVAAPSVFIFPPSDE | |
| | CDRs | QLKSGTASVVCLLNNFYPREAKVQWKVDNALQSGNSQESVT | |
| | Underlined | EQDSKDSTYSLSSTLTLSKADYEKHKVYACEVTHQGLSSPV | |
| | & Bold | TKSENRGEC | |

### TGFβ TRAP

In certain aspects, provided herein are methods of treating cancer comprising administering to a subject having cancer an antibody, or functional fragment or functional variant thereof, that specifically binds programmed death protein 1 (PD1) e.g., described herein, in combination with a fusion protein that comprises a targeting moiety and an immunomodulatory moiety, wherein: i) said targeting moiety specifically binds epidermal growth factor receptor (EGFR); and (ii) said immunomodulatory moiety comprises an amino acid sequence of the extracellular domain (ECD) of transforming growth factor-beta receptor II (TGFβRII).

In some embodiments, the TGFβRII ECD binds to at least one TGFβ isoform. In some embodiments, the TGFβRII ECD binds to TGFβ1. In some embodiments, the TGFβRII ECD binds to TGFβ3. In some embodiments, the TGFβRII ECD does not bind to TGFβ2.

In some embodiments, the TGFβRII ECD comprises sufficient sequence of a naturally occurring TGFβRII ECD to enable the protein to bind TGFβ. In some embodiments, the TGFβRII ECD comprises sufficient sequence of a naturally occurring TGFβRII ECD to enable the protein to bind TGFβ1. In some embodiments, the TGFβRII ECD comprises sufficient sequence of a naturally occurring TGFβRII ECD to enable the protein to bind TGFβ3.

In some embodiments, the extracellular domain of TGFβRII comprises a truncated portion of SEQ ID NO: 56, that is capable of binding TGFβ. The extracellular domain of TGFβRII may be truncated on the N-terminus, the C-terminus, or both the N and C terminus. The truncation may comprise the deletion of 1-10 amino acids. The truncation may comprise the deletion of 1, 2, 3, 4, 5, 6, 7, 8, 9, or 10 amino acids. The truncation may comprise the deletion of 1, 2, 3, 4, 5 amino acids from the N terminus, the C terminus, or both the N and C terminus.

In some embodiments, the extracellular domain of TGFβRII comprises an amino acid sequence at least 90%, 91%, 92%, 93%, 94%, 95%, 96%, 97%, 98%, 99%, or 100% identical to the amino acid sequence of SEQ ID NO: 56. In some embodiments, the extracellular domain of TGFβRII consists essentially of an amino acid sequence at least 90%, 91%, 92%, 93%, 94%, 95%, 96%, 97%, 98%, 99%, or 100% identical to the amino acid sequence of SEQ ID NO: 56. In some embodiments, the extracellular domain of TGFβRII consists of an amino acid sequence at least 90%, 91%, 92%, 93%, 94%, 95%, 96%, 97%, 98%, 99%, or 100% identical to the amino acid sequence of SEQ ID NO: 56.

TABLE 3
Exemplary TGFβRII ECD

| | | SEQ |
|---|---|---|
| | | ID |
| | Amino Acid Sequence | NO |
| TGFβRII | TIPPHVQKSVNNDMIVTDNNGA | 56 |
| ECD | VKFPQLCKFCDVRFSTCDNQKS | |
| | CMSNCSITSICEKPQEVCVAVW | |
| | RKNDENITLETVCHDPKLPYHD | |
| | FILEDAASPKCIMKEKKKPGET | |
| | FFMCSCSSDECNDNIIFSEEYN | |
| | TSNPD | |

### Orientation

In some embodiments, the immunomodulatory moiety is operably connected to the C terminus of the targeting moiety. In some embodiments, the immunomodulatory moiety is operably connected to the N terminus of the targeting moiety.

In some embodiments, the targeting moiety is an antibody (or functional fragment or variant thereof) that comprises 1) a VH or a heavy chain, and 2) a VL or a light chain. In some embodiments, the immunomodulatory moiety is operably connected to the C terminus of the VH or heavy chain. In some embodiments, the immunomodulatory moiety is operably connected to the C terminus of the VL or light chain. In some embodiments, the immunomodulatory moiety is operably connected to the C terminus of the constant region of the heavy chain. In some embodiments, the immunomodulatory moiety is operably connected to the C terminus of the constant region of the light chain. In some embodiments, the immunomodulatory moiety is operably connected to the N terminus of the VH or heavy chain. In some embodiments, the immunomodulatory moiety is operably connected to the N terminus of the VL or light chain.

### Linkers

In some embodiments, the targeting moiety and an immunomodulatory moiety of the fusion protein are directly operably connected. In some embodiments, the targeting moiety and an immunomodulatory moiety of the fusion protein are indirectly operably connected. In some embodiments, the targeting moiety and an immunomodulatory moiety of the fusion protein are indirectly operably connected via a linker. In some embodiments, the linker is a peptide linker.

Any suitable peptide linker known in the art can be used that enables the immunomodulatory moiety and the targeting moiety to bind their respective antigens. Exemplary peptide linkers comprising glycine and serine amino acids are provided in Table 4.

In some embodiments, the linker comprises an amino acid sequence at least 95%, 96%, 97%, 98%, 99%, or 100% identical to the amino acid sequence of any one of SEQ ID NOS: 57-61. In some embodiments, the linker comprises the amino acid sequence of any one of SEQ ID NOS: 57-61, or the amino acid sequence of any one of SEQ ID NOS: 57-61 with 1, 2, or 3 amino acid modifications.

In some embodiments, the linker comprises an amino acid sequence at least 95%, 96%, 97%, 98%, 99%, or 100% identical to the amino acid sequence of SEQ ID NO: 57. In some embodiments, the linker comprises an amino acid sequence 100% identical to the amino acid sequence of SEQ ID NO: 57. In some embodiments, the linker comprises the amino acid sequence of SEQ ID NO: 57, or the amino acid sequence of SEQ ID NO: 57 with 1, 2, or 3 amino acid modifications. In some embodiments, the linker consists essentially of an amino acid sequence 100% identical to the amino acid sequence of SEQ ID NO: 57. In some embodiments, the linker consists of an amino acid sequence 100% identical to the amino acid sequence of SEQ ID NO: 57. In some embodiments, the linker consists of the amino acid sequence of SEQ ID NO: 57, or the amino acid sequence of SEQ ID NO: 57 with 1, 2, or 3 amino acid modifications.

In some embodiments, the linker comprises an amino acid sequence at least 95%, 96%, 97%, 98%, 99%, or 100% identical to the amino acid sequence of SEQ ID NO: 58. In some embodiments, the linker comprises an amino acid sequence 100% identical to the amino acid sequence of SEQ ID NO: 58. In some embodiments, the linker comprises the amino acid sequence of SEQ ID NO: 58, or the amino acid sequence of SEQ ID NO: 28 with 1, 2, or 3 amino acid modifications. In some embodiments, the linker consists essentially of an amino acid sequence 100% identical to the amino acid sequence of SEQ ID NO: 58. In some embodiments, the linker consists of an amino acid sequence 100% identical to the amino acid sequence of SEQ ID NO: 58. In some embodiments, the linker consists of the amino acid sequence of SEQ ID NO: 58, or the amino acid sequence of SEQ ID NO: 58 with 1, 2, or 3 amino acid modifications.

In some embodiments, the linker comprises an amino acid sequence at least 95%, 96%, 97%, 98%, 99%, or 100% identical to the amino acid sequence of SEQ ID NO: 59. In some embodiments, the linker comprises an amino acid sequence 100% identical to the amino acid sequence of SEQ ID NO: 59. In some embodiments, the linker comprises the amino acid sequence of SEQ ID NO: 59, or the amino acid sequence of SEQ ID NO: 59 with 1, 2, or 3 amino acid modifications. In some embodiments, the linker consists essentially of an amino acid sequence 100% identical to the amino acid sequence of SEQ ID NO: 59. In some embodiments, the linker consists of an amino acid sequence 100% identical to the amino acid sequence of SEQ ID NO: 59. In some embodiments, the linker consists of the amino acid sequence of SEQ ID NO: 59, or the amino acid sequence of SEQ ID NO: 59 with 1, 2, or 3 amino acid modifications.

In some embodiments, the linker comprises an amino acid sequence at least 95%, 96%, 97%, 98%, 99%, or 100% identical to the amino acid sequence of SEQ ID NO: 60. In some embodiments, the linker comprises an amino acid sequence 100% identical to the amino acid sequence of SEQ ID NO: 60. In some embodiments, the linker comprises the amino acid sequence of SEQ ID NO: 60, or the amino acid sequence of SEQ ID NO: 60 with 1, 2, or 3 amino acid modifications. In some embodiments, the linker consists essentially of an amino acid sequence 100% identical to the amino acid sequence of SEQ ID NO: 60. In some embodiments, the linker consists of an amino acid sequence 100% identical to the amino acid sequence of SEQ ID NO: 60. In some embodiments, the linker consists of the amino acid sequence of SEQ ID NO: 60, or the amino acid sequence of SEQ ID NO: 60 with 1, 2, or 3 amino acid modifications.

In some embodiments, the linker comprises an amino acid sequence at least 95%, 96%, 97%, 98%, 99%, or 100% identical to the amino acid sequence of SEQ ID NO: 61. In some embodiments, the linker comprises an amino acid sequence 100% identical to the amino acid sequence of SEQ ID NO: 61. In some embodiments, the linker comprises the amino acid sequence of SEQ ID NO: 61, or the amino acid sequence of SEQ ID NO: 61 with 1, 2, or 3 amino acid modifications. In some embodiments, the linker consists essentially of an amino acid sequence 100% identical to the amino acid sequence of SEQ ID NO: 61. In some embodiments, the linker consists of an amino acid sequence 100% identical to the amino acid sequence of SEQ ID NO: 61. In some embodiments, the linker consists of the amino acid sequence of SEQ ID NO: 61, or the amino acid sequence of SEQ ID NO: 61 with 1, 2, or 3 amino acid modifications.

TABLE 4
Exemplary Linkers

| Linker | Amino Acid Sequence | SEQ ID NO |
|---|---|---|
| (GGGS)₃ | GGGGSGGGGSGGGGS | 57 |
| (GGGS)₄ | GGGGSGGGGSGGGGSGGGS | 58 |
| (GGGS)₅ | GGGGSGGGGSGGGGSGGGSGGGS | 59 |
| (GGGS)₂ | GGGGSGGGGS | 60 |
| (GGGS)₁ | GGGGS | 61 |

### Exemplary Fusion Proteins

Exemplary fusion proteins of the present disclosure are provided in Table 5. In one embodiment, the fusion protein comprises BCA101. BCA101, is a bifunctional fusion protein that comprises an anti-EGFR antibody and the extracellular domain of TGFβRII fused to the C-terminus of the anti-EGFR antibody light chain.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 43. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 62. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 43; and a light chain that comprises an amino acid sequence at least 90%, 91%, 92%, 93%, 94%, 95%, 96%, 97%, 98%, 99%, or 100% identical to the amino acid sequence of SEQ ID NO: 62.

In some embodiments, the fusion protein comprises a heavy chain that comprises an amino acid sequence 100% identical to the amino acid sequence of SEQ ID NO: 43. In some embodiments, the fusion protein comprises a light chain that comprises an amino acid sequence 100% identical to the amino acid sequence of SEQ ID NO: 62. In some embodiments, the fusion protein comprises a heavy chain that comprises an amino acid sequence 100% identical to the amino acid sequence of SEQ ID NO: 43; and a light chain that comprises an amino acid sequence 100% identical to the amino acid sequence of SEQ ID NO: 62.

In some embodiments, the fusion protein comprises a heavy chain, wherein the amino acid sequence of the heavy chain comprises the amino acid sequence of SEQ ID NO: 43. In some embodiments, the fusion protein comprises a light chain, wherein the amino acid sequence of the light chain comprises the amino acid sequence of SEQ ID NO: 62. In some embodiments, the fusion protein comprises a heavy chain, wherein the amino acid sequence of the heavy chain comprises the amino acid sequence of SEQ ID NO: 43; and a light chain, wherein the amino acid sequence of the light chain comprises the amino acid sequence of SEQ ID NO: 62.

In some embodiments, the fusion protein comprises a heavy chain that comprises the amino acid sequence of SEQ ID NO: 43, with 1, 2, or 3 amino acid modifications. In some embodiments, the fusion protein comprises a light chain that comprises the amino acid sequence of SEQ ID NO: 62 with 1, 2, or 3 amino acid modifications. In some embodiments, the fusion protein comprises a heavy chain that comprises the amino acid sequence of SEQ ID NO: 43, with 1, 2, or 3 amino acid modifications; and a light chain that comprises the amino acid sequence of SEQ ID NO: 62 with 1, 2, or 3 amino acid modifications.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 43. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 62. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 43; and a light chain that consists of an amino acid sequence at least 90%, 91%, 92%, 93%, 94%, 95%, 96%, 97%, 98%, 99%, or 100% identical to the amino acid sequence of SEQ ID NO: 62.

In some embodiments, the fusion protein comprises a heavy chain that consists of an amino acid sequence 100% identical to the amino acid sequence of SEQ ID NO: 43. In some embodiments, the fusion protein comprises a light chain that consists of an amino acid sequence 100% identical to the amino acid sequence of SEQ ID NO: 62. In some embodiments, the fusion protein comprises a heavy chain that consists of an amino acid sequence 100% identical to the amino acid sequence of SEQ ID NO: 43; and a light chain that comprises an amino acid sequence 100% identical to the amino acid sequence of SEQ ID NO: 62.

In some embodiments, the fusion protein comprises a heavy chain that consists of the amino acid sequence of SEQ ID NO: 43, with 1, 2, or 3 amino acid modifications. In some embodiments, the fusion protein comprises a light chain that consists of the amino acid sequence of SEQ ID NO: 62, with 1, 2, or 3 amino acid modifications. In some embodiments, the fusion protein comprises a heavy chain that consists of the amino acid sequence of SEQ ID NO: 43, with 1, 2, or 3 amino acid modifications; and a light chain that consists of the amino acid sequence of SEQ ID NO: 62, with 1, 2, or 3 amino acid modifications.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 63. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 44. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 63; and a light chain that comprises an amino acid sequence at least 90%, 91%, 92%, 93%, 94%, 95%, 96%, 97%, 98%, 99%, or 100% identical to the amino acid sequence of SEQ ID NO: 44.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 63. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 44. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 63; and a light chain that consists of an amino acid sequence at least 90%, 91%, 92%, 93%, 94%, 95%, 96%, 97%, 98%, 99%, or 100% identical to the amino acid sequence of SEQ ID NO: 44.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 42. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 62. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 42; and a light chain that comprises an amino acid sequence at least 90%, 91%, 92%, 93%, 94%, 95%, 96%, 97%, 98%, 99%, or 100% identical to the amino acid sequence of SEQ ID NO: 62.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 42. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 62. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO:42; and a light chain that consists of an amino acid sequence at least 90%, 91%, 92%, 93%, 94%, 95%, 96%, 97%, 98%, 99%, or 100% identical to the amino acid sequence of SEQ ID NO: 62.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 64. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 44. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 64; and a light chain that comprises an amino acid sequence at least 90%, 91%, 92%, 93%, 94%, 95%, 96%, 97%, 98%, 99%, or 100% identical to the amino acid sequence of SEQ ID NO: 44.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 64. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 44. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 64; and a light chain that consists of an amino acid sequence at least 90%, 91%, 92%, 93%, 94%, 95%, 96%, 97%, 98%, 99%, or 100% identical to the amino acid sequence of SEQ ID NO: 44.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 53. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 65. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 53; and a light chain that comprises an amino acid sequence at least 90%, 91%, 92%, 93%, 94%, 95%, 96%, 97%, 98%, 99%, or 100% identical to the amino acid sequence of SEQ ID NO: 65.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 53. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 65. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 53; and a light chain that consists of an amino acid sequence at least 90%, 91%, 92%, 93%, 94%, 95%, 96%, 97%, 98%, 99%, or 100% identical to the amino acid sequence of SEQ ID NO: 65.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 66. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 55. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 66; and a light chain that comprises an amino acid sequence at least 90%, 91%, 92%, 93%, 94%, 95%, 96%, 97%, 98%, 99%, or 100% identical to the amino acid sequence of SEQ ID NO: 55.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 66. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 55. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 66; and a light chain that consists of an amino acid sequence at least 90%, 91%, 92%, 93%, 94%, 95%, 96%, 97%, 98%, 99%, or 100% identical to the amino acid sequence of SEQ ID NO: 55.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 54. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 65. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 54; and a light chain that comprises an amino acid sequence at least 90%, 91%, 92%, 93%, 94%, 95%, 96%, 97%, 98%, 99%, or 100% identical to the amino acid sequence of SEQ ID NO: 65.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 54. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 65. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 54; and a light chain that consists of an amino acid sequence at least 90%, 91%, 92%, 93%, 94%, 95%, 96%, 97%, 98%, 99%, or 100% identical to the amino acid sequence of SEQ ID NO: 65.

In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 67. In some embodiments, the fusion protein comprises a light chain that comprises an amino acid sequence at least 90%, 91%, 92%, 93%, 94%, 95%, 96%, 97%, 98%, 99%, or 100% identical to the amino acid sequence of SEQ ID NO: 55. In some embodiments, the fusion protein comprises a heavy chain that comprises an amino acid sequence at least 90%, 91%, 92%, 93%, 94%, 95%, 96%, 97%, 98%, 99%, or 100% identical to the amino acid sequence of SEQ ID NO: 67; and a light chain that comprises an amino acid sequence at least 90%, 91%, 92%, 93%, 94%, 95%, 96%, 97%, 98%, 99%, or 100% identical to the amino acid sequence of SEQ ID NO: 55.

In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 67. In some embodiments, the fusion protein comprises a light chain that consists of an amino acid sequence at least 90%, 91%, 92%, 93%, 94%, 95%, 96%, 97%, 98%, 99%, or 100% identical to the amino acid sequence of SEQ ID NO: 55. In some embodiments, the fusion protein comprises a heavy chain that consists of an amino acid sequence at least 90%, 91%, 92%, 93%, 94%, 95%, 96%, 97%, 98%, 99%, or 100% identical to the amino acid sequence of SEQ ID NO: 67; and a light chain that consists of an amino acid sequence at least 90%, 91%, 92%, 93%, 94%, 95%, 96%, 97%, 98%, 99%, or 100% identical to the amino acid sequence of SEQ ID NO: 55.

TABLE 5
Exemplary Fusion Proteins

| | | | SEQ ID |
|---|---|---|---|
| Fusion | Component | Amino Acid Sequence | NO |
| BCA101 | Heavy Chain | QVQLKQSGPGLVQPSQSLSITCTVSGFSLTNYGVHWVRQS | 43 |
| | Anti-EGFR heavy | PGKGLEWLGVIWSGGNTDYNTPFTSRLSINKDNSKSQVFF | |
| | chain | KMNSLQSNDTAIYYCARALTYYDYEFAYWGQGTLVTVSAA | |
| | VH Underlined | STKGPSVFPLAPSSKSTSGGTAALGCLVKDYFPEPVTVSW | |
| | CDRs Underlined | NSGALTSGVHTFPAVLQSSGLYSLSSVVTVPSSSLGTQTY | |
| | & Bold | ICNVNHKPSNTKVDKRVEPKSCDKTHTCPPCPAPELLGGP | |
| | | SVFLFPPKPKDTLMISRTPEVTCVVVDVSHEDPEVKENWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPG | |
| | Light Chain | DILLTQSPVILSVSPGERVSFSCRASQSIGTNIHWYQQRT | 62 |
| | Anti-EGFR light | NGSPRLLIKYASESISGIPSRFSGSGSGTDFTLSINSVES | |
| | chain | EDIADYYCQQNNNWPTTFGAGTKLELKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSFNRGECGGGGSGGGGSGGGGSTIPPHVQKSVN | |
| | Linker | NDMIVTDNNGAVKFPQLCKFCDVRFSTCDNQKSCMSNCSI | |
| | italicized | TSICEKPQEVCVAVWRKNDENITLETVCHDPKLPYHDFIL | |
| | TGFβR ECD Bold | EDAASPKCIMKEKKKPGETFFMCSCSSDECNDNIIFSEEY | |
| | | NTSNPD | |
| Heavy Chain | Heavy Chain | QVQLKQSGPGLVQPSQSLSITCTVSGFSLTNYGVHWVRQS | 63 |
| Fusion | Anti-EGFR heavy | PGKGLEWLGVIWSGGNTDYNTPFTSRLSINKDNSKSQVFF | |
| | chain | KMNSLQSNDTAIYYCARALTYYDYEFAYWGQGTLVTVSAA | |
| | VH Underlined | STKGPSVFPLAPSSKSTSGGTAALGCLVKDYFPEPVTVSW | |
| | CDRs Underlined | NSGALTSGVHTFPAVLQSSGLYSLSSVVTVPSSSLGTQTY | |
| | & Bold | ICNVNHKPSNTKVDKRVEPKSCDKTHTCPPCPAPELLGGP | |
| | Linker | SVFLFPPKPKDTLMISRTPEVTCVVVDVSHEDPEVKENWY | |
| | italicized | VDGVEVHNAKTKPREEQYNSTYRVVSVLTVLHQDWLNGKE | |
| | TGFβR ECD Bold | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGGGGGSGGGGSGGGGSTIPPHVQKSVNNDMIVT | |
| | | DNNGAVKFPQLCKFCDVRFSTCDNQKSCMSNCSITSICEK | |
| | | PQEVCVAVWRKNDENITLETVCHDPKLPYHDFILEDAASP | |
| | | KCIMKEKKKPGETFFMCSCSSDECNDNIIFSEEYNTSNPD | |
| | Light Chain | DILLTQSPVILSVSPGERVSFSCRASQSIGTNIHWYQQRT | 44 |
| | Anti-EGFR light | NGSPRLLIKYASESISGIPSRFSGSGSGTDFTLSINSVES | |
| | chain | EDIADYYCQQNNNWPTTFGAGTKLELKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSENRGEC | |
| Cetuximab | Heavy Chain | QVQLKQSGPGLVQPSQSLSITCTVSGFSLTNYGVHWVRQS | 42 |
| Light Chain | Anti-EGFR heavy | PGKGLEWLGVIWSGGNTDYNTPFTSRLSINKDNSKSQVFF | |
| Fusion | chain | KMNSLQSNDTAIYYCARALTYYDYEFAYWGQGTLVTVSAA | |
| | VH Underlined | STKGPSVFPLAPSSKSTSGGTAALGCLVKDYFPEPVTVSW | |
| | CDRs Underlined | NSGALTSGVHTFPAVLQSSGLYSLSSVVTVPSSSLGTQTY | |
| | & Bold | ICNVNHKPSNTKVDKRVEPKSCDKTHTCPPCPAPELLGGP | |
| | | SVFLFPPKPKDTLMISRTPEVTCVVVDVSHEDPEVKENWY | |
| | | VDGVEVHNAKTKPREEQYNSTYRVVSVLTVLHQDWLNGKE | |
| | | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGK | |
| | Light Chain | DILLTQSPVILSVSPGERVSFSCRASQSIGTNIHWYQQRT | 62 |
| | Anti-EGFR light | NGSPRLLIKYASESISGIPSRFSGSGSGTDETLSINSVES | |
| | chain | EDIADYYCQQNNNWPTTFGAGTKLELKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSFNRGECGGGGSGGGGSGGGGSTIPPHVQKSVN | |
| | Linker | NDMIVTDNNGAVKFPQLCKFCDVRFSTCDNQKSCMSNCSI | |
| | italicized | TSICEKPQEVCVAVWRKNDENITLETVCHDPKLPYHDFIL | |
| | TGFβR ECD Bold | EDAASPKCIMKEKKKPGETFFMCSCSSDECNDNIIFSEEY | |
| | | NTSNPD | |
| Cetuximab | Heavy Chain | QVQLKQSGPGLVQPSQSLSITCTVSGFSLTNYGVHWVRQS | 64 |
| Heavy Chain | Anti-EGFR heavy | PGKGLEWLGVIWSGGNTDYNTPFTSRLSINKDNSKSQVFF | |
| Fusion | chain | KMNSLQSNDTAIYYCARALTYYDYEFAYWGQGTLVTVSAA | |
| | VH Underlined | STKGPSVFPLAPSSKSTSGGTAALGCLVKDYFPEPVTVSW | |
| | CDRs Underlined | NSGALTSGVHTFPAVLQSSGLYSLSSVVTVPSSSLGTQTY | |
| | & Bold | ICNVNHKPSNTKVDKRVEPKSCDKTHTCPPCPAPELLGGP | |
| | Linker | SVFLFPPKPKDTLMISRTPEVTCVVVDVSHEDPEVKENWY | |
| | italicized | VDGVEVHNAKTKPREEQYNSTYRVVSVLTVLHQDWLNGKE | |
| | TGEβR ECD Bold | YKCKVSNKALPAPIEKTISKAKGQPREPQVYTLPPSRDEL | |
| | | TKNQVSLTCLVKGFYPSDIAVEWESNGQPENNYKTTPPVL | |
| | | DSDGSFFLYSKLTVDKSRWQQGNVFSCSVMHEALHNHYTQ | |
| | | KSLSLSPGKGGGGSGGGGSGGGGSTIPPHVQKSVNNDMIV | |
| | | TDNNGAVKFPQLCKFCDVRESTCDNQKSCMSNCSITSICE | |
| | | KPQEVCVAVWRKNDENITLETVCHDPKLPYHDFILEDAAS | |
| | | PKCIMKEKKKPGETFFMCSCSSDECNDNIIFSEEYNTSNP | |
| | | D | |
| | Light Chain | DILLTQSPVILSVSPGERVSFSCRASQSIGTNIHWYQQRT | 44 |
| | Anti-EGFR light | NGSPRLLIKYASESISGIPSRFSGSGSGTDFTLSINSVES | |
| | chain | EDIADYYCQQNNNWPTTFGAGTKLELKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSENRGEC | |
| Panitumumab | Heavy Chain | QVQLQESGPGLVKPSETLSLTCTVSGGSVSSGDYYWTWIR | 53 |
| Light Chain | Anti-EGFR heavy | QSPGKGLEWIGHIYYSGNTNYNPSLKSRLTISIDTSKTQF | |
| Fusion (with | chain | SLKLSSVTAADTAIYYCVRDRVTGAFDIWGQGTMVTVSSA | |
| HC C | VH Underlined | STKGPSVFPLAPCSRSTSESTAALGCLVKDYFPEPVTVSW | |
| terminal | CDRs Underlined | NSGALTSGVHTFPAVLQSSGLYSLSSVVTVPSSNFGTQTY | |
| Lysine) | & Bold | TCNVDHKPSNTKVDKTVERKCCVECPPCPAPPVAGPSVEL | |
| | | FPPKPKDTLMISRTPEVTCVVVDVSHEDPEVQFNWYVDGV | |
| | | EVHNAKTKPREEQFNSTFRVVSVLTVVHQDWLNGKEYKCK | |
| | | VSNKGLPAPIEKTISKTKGQPREPQVYTLPPSREEMTKNQ | |
| | | VSLTCLVKGFYPSDIAVEWESNGQPENNYKTTPPMLDSDG | |
| | | SFFLYSKLTVDKSRWQQGNVFSCSVMHEALHNHYTQKSLS | |
| | | LSPGK | |
| | Light Chain | DIQMTQSPSSLSASVGDRVTITCQASQDISNYLNWYQQKP | 65 |
| | Anti-EGFR light | GKAPKLLIYDASNLETGVPSRFSGSGSGTDFTFTISSLQP | |
| | chain | EDIATYFCQHFDHLPLAFGGGTKVEIKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSFNRGECGGGGSGGGGSGGGGSTIPPHVQKSVN | |
| | Linker | NDMIVTDNNGAVKFPQLCKFCDVRFSTCDNQKSCMSNCSI | |
| | italicized | TSICEKPQEVCVAVWRKNDENITLETVCHDPKLPYHDFIL | |
| | TGFβR ECD Bold | EDAASPKCIMKEKKKPGETFFMCSCSSDECNDNIIFSEEY | |
| | | NTSNPD | |
| Panitumumab | Heavy Chain | QVQLQESGPGLVKPSETLSLTCTVSGGSVSSGDYYWTWIR | 66 |
| Heavy Chain | Anti-EGFR heavy | QSPGKGLEWIGHIYYSGNTNYNPSLKSRLTISIDTSKTQF | |
| fusion (with | chain | SLKLSSVTAADTAIYYCVRDRVTGAFDIWGQGTMVTVSSA | |
| HC C | VH Underlined | STKGPSVFPLAPCSRSTSESTAALGCLVKDYFPEPVTVSW | |
| terminal | CDRs Underlined | NSGALTSGVHTFPAVLQSSGLYSLSSVVTVPSSNFGTQTY | |
| Lysine) | & Bold | TCNVDHKPSNTKVDKTVERKCCVECPPCPAPPVAGPSVEL | |
| | Linker | FPPKPKDTLMISRTPEVTCVVVDVSHEDPEVQFNWYVDGV | |
| | italicized | EVHNAKTKPREEQFNSTFRVVSVLTVVHQDWLNGKEYKCK | |
| | TGFβR ECD Bold | VSNKGLPAPIEKTISKTKGQPREPQVYTLPPSREEMTKNQ | |
| | | VSLTCLVKGFYPSDIAVEWESNGQPENNYKTTPPMLDSDG | |
| | | SFFLYSKLTVDKSRWQQGNVFSCSVMHEALHNHYTQKSLS | |
| | | LSPGKGGGGSGGGGSGGGGSTIPPHVQKSVNNDMIVTDNN | |
| | | GAVKFPQLCKFCDVRFSTCDNQKSCMSNCSITSICEKPQE | |
| | | VCVAVWRKNDENITLETVCHDPKLPYHDFILEDAASPKCI | |
| | | MKEKKKPGETFFMCSCSSDECNDNIIFSEEYNTSNPD | |
| | Light Chain | DIQMTQSPSSLSASVGDRVTITCQASQDISNYLNWYQQKP | 55 |
| | Anti-EGFR light | GKAPKLLIYDASNLETGVPSRFSGSGSGTDFTFTISSLQP | |
| | chain | EDIATYFCQHFDHLPLAFGGGTKVEIKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSENRGEC | |
| Panitumumab | Heavy Chain | QVQLQESGPGLVKPSETLSLTCTVSGGSVSSGDYYWTWIR | 54 |
| Light Chain | Anti-EGFR heavy | QSPGKGLEWIGHIYYSGNTNYNPSLKSRLTISIDTSKTQF | |
| Fusion (with | chain | SLKLSSVTAADTAIYYCVRDRVTGAFDIWGQGTMVTVSSA | |
| HC C | VH Underlined | STKGPSVFPLAPCSRSTSESTAALGCLVKDYFPEPVTVSW | |
| terminal | CDRs Underlined | NSGALTSGVHTFPAVLQSSGLYSLSSVVTVPSSNFGTQTY | |
| Lysin) | & Bold | TCNVDHKPSNTKVDKTVERKCCVECPPCPAPPVAGPSVFL | |
| | | FPPKPKDTLMISRTPEVTCVVVDVSHEDPEVQFNWYVDGV | |
| | | EVHNAKTKPREEQFNSTFRVVSVLTVVHQDWLNGKEYKCK | |
| | | VSNKGLPAPIEKTISKTKGQPREPQVYTLPPSREEMTKNQ | |
| | | VSLTCLVKGFYPSDIAVEWESNGQPENNYKTTPPMLDSDG | |
| | | SFFLYSKLTVDKSRWQQGNVFSCSVMHEALHNHYTQKSLS | |
| | | LSPG | |
| | Light Chain | DIQMTQSPSSLSASVGDRVTITCQASQDISNYLNWYQQKP | 65 |
| | Anti-EGFR light | GKAPKLLIYDASNLETGVPSRFSGSGSGTDFTFTISSLQP | |
| | chain | EDIATYFCQHFDHLPLAFGGGTKVEIKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSFNRGECGGGGSGGGGSGGGGSTIPPHVQKSVN | |
| | Linker | NDMIVTDNNGAVKFPQLCKFCDVRFSTCDNQKSCMSNCSI | |
| | italicized | TSICEKPQEVCVAVWRKNDENITLETVCHDPKLPYHDFIL | |
| | TGFβR ECD Bold | EDAASPKCIMKEKKKPGETFFMCSCSSDECNDNIIFSEEY | |
| | | NTSNPD | |
| Panitumumab | Heavy Chain | QVQLQESGPGLVKPSETLSLTCTVSGGSVSSGDYYWTWIR | 67 |
| Heavy Chain | Anti-EGFR heavy | QSPGKGLEWIGHIYYSGNTNYNPSLKSRLTISIDTSKTQF | |
| fusion (with | chain | SLKLSSVTAADTAIYYCVRDRVTGAFDIWGQGTMVTVSSA | |
| HC C | VH Underlined | STKGPSVFPLAPCSRSTSESTAALGCLVKDYFPEPVTVSW | |
| terminal | CDRs Underlined | NSGALTSGVHTFPAVLQSSGLYSLSSVVTVPSSNFGTQTY | |
| Lysine) | & Bold | TCNVDHKPSNTKVDKTVERKCCVECPPCPAPPVAGPSVFL | |
| | Linker | FPPKPKDTLMISRTPEVTCVVVDVSHEDPEVQFNWYVDGV | |
| | italicized | EVHNAKTKPREEQFNSTFRVVSVLTVVHQDWLNGKEYKCK | |
| | TGFβR ECD Bold | VSNKGLPAPIEKTISKTKGQPREPQVYTLPPSREEMTKNQ | |
| | | VSLTCLVKGFYPSDIAVEWESNGQPENNYKTTPPMLDSDG | |
| | | SFFLYSKLTVDKSRWQQGNVFSCSVMHEALHNHYTQKSLS | |
| | | LSPGGGGGGSGGGGSGGGGSTIPPHVQKSVNNDMIVTDNN | |
| | | GAVKFPQLCKFCDVRFSTCDNQKSCMSNCSITSICEKPQE | |
| | | VCVAVWRKNDENITLETVCHDPKLPYHDFILEDAASPKCI | |
| | | MKEKKKPGETFFMCSCSSDECNDNIIFSEEYNTSNPD | |
| | Light Chain | DIQMTQSPSSLSASVGDRVTITCQASQDISNYLNWYQQKP | 55 |
| | Anti-EGFR light | GKAPKLLIYDASNLETGVPSRFSGSGSGTDFTFTISSLQP | |
| | chain | EDIATYFCQHFDHLPLAFGGGTKVEIKRTVAAPSVFIFPP | |
| | VL Underlined | SDEQLKSGTASVVCLLNNFYPREAKVQWKVDNALQSGNSQ | |
| | CDRs Underlined | ESVTEQDSKDSTYSLSSTLTLSKADYEKHKVYACEVTHQG | |
| | & Bold | LSSPVTKSENRGEC | |

### Methods of Making Fusion Proteins

Fusion proteins described herein can be made by any conventional technique known in the art, for example, recombinant techniques or chemical synthesis (e.g., solid phase peptide synthesis). In one embodiments, the fusion protein is made through recombinant expression in a cell. Briefly, the fusion protein can be made by synthesizing the DNA encoding the fusion protein and cloning the DNA into any suitable expression vector. Numerous cloning vectors are known to those of skill in the art, and the selection of an appropriate cloning vector is a matter of choice. The gene can be placed under the control of a promoter, ribosome binding site (for bacterial expression) and, optionally, an operator, so that the DNA sequence encoding the fusion protein is transcribed into RNA in the host cell transformed by a vector containing this expression construction. The coding sequence may or may not contain a signal peptide or leader sequence. Heterologous leader sequences can be added to the coding sequence that causes the secretion of the expressed polypeptide from the host organism. Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences. The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

The expression vector may then used to transform an appropriate host cell. A number of mammalian cell lines are known in the art and include immortalized cell lines available from the American Type Culture Collection (ATCC), such as, but not limited to, Chinese hamster ovary (CHO) cells, CHO-suspension cells (CHO-S), HeLa cells, HEK293, baby hamster kidney (BHK) cells, monkey kidney cells (COS), VERO, HepG2, MadinDarby bovine kidney (MDBK) cells, NOS, U2OS, A549, HT1080, CAD, P19, NIH3T3, L929, N2a, MCF-7, Y79, SO-Rb50, DUKX-X11, and J558L. In some embodiments, the fusion protein is produced in CHO or CHO-S cells.

Depending on the expression system and host selected, the fusion protein is produced by growing host cells transformed by an expression vector described above under conditions whereby the fusion protein is expressed. The fusion protein is then isolated from the host cells and purified. If the expression system secretes the fusion protein into growth media, the fusion protein can be purified directly from the media. If the fusion protein is not secreted, it is isolated from cell lysates. The selection of the appropriate growth conditions and recovery methods are within the skill of the art. Once purified, the amino acid sequences of the fusion proteins can be determined, i.e., by repetitive cycles of Edman degradation, followed by amino acid analysis by HPLC. Other methods of amino acid sequencing are also known in the art. Once purified, the functionality of the fusion protein can be assessed, e.g., as described herein, e.g., utilizing a bifunctional ELISA.

As described above, functionality of the fusion protein can be tested by any method known in the art, e.g., ELISA. Each functionality can be measured in a separate assay, e.g., TGFβ binding and EGFR binding can be measured in two separate ELISAs. For example, an ELISA plate can be coated with EGFR Fc chimera, and used to evaluate EGFR binding; and a separate ELISA plate can be coated with TGFβ-1 to evaluate TGFβ-1 binding. Both functionalities can also be evaluated in a bifunctional ELISA. For example, an anti-idiotype mAb against cetuximab (for use with BCA101 fusion protein) can be used to capture BCA101 and the bound BCA101 can be detected by an enzyme-labeled polyclonal antibody against TGFβRIIECD. The concentration of BCA101 in samples can be back-calculated from a BCA101 calibration curve. Target binding can also be evaluated via Biocore, wherein EGFR and TGFβ1 targets are immobilized on activated CM5 chips and then incubated with serial concentrations of the fusion protein. Additional in vitro functional assays can also be performed to evaluate the fusion proteins, including for example, cell surface binding by flow cytometry, inhibition of cell proliferation, ADCC assay, neutralization of TGFβ1 induced IL-11 release; neutralization of TGFβ1 induced SMAD signaling.

### Methods of Use

In one aspect, provided herein are methods of treating cancer in a subject by administering to the subject having cancer an agent that specifically binds PD1 (e.g., an agent described herein) and a fusion protein that comprises a targeting moiety and an immunomodulatory moiety, wherein: i) said targeting moiety specifically binds epidermal growth factor receptor (EGFR); and (ii) said immunomodulatory moiety comprises an amino acid sequence of the extracellular domain of transforming growth factor-beta receptor II (TGFβRII).

In some embodiments, the methods disclosed herein are used in place of standard of care therapies. In certain embodiments, a standard of care therapy is used in combination with any method disclosed herein. Standard-of-care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard-of-care treatments for a wide variety of cancers. In some embodiments, the methods disclosed herein are used after standard of care therapy has failed.

In some embodiments, the fusion protein is co-administered, administered prior to, or administered after, the antibody, or functional fragment or functional variant thereof, that specifically binds PD1.

In some embodiments, the fusion protein is co-administered with the antibody, or functional fragment or functional variant thereof, that specifically binds PD1. In some embodiments, the fusion protein is administered simultaneously with the antibody, or functional fragment or functional variant thereof, that specifically binds PD1. In some embodiments, the fusion protein is administered within 30 minutes, 1 hour, 2 hours, 3 hours, 4 hours, 5 hours, 6 hours, 7 hours, 8 hours, 9 hours, 10 hours, 11 hours, or 12 hours of administration of the antibody, or functional fragment or functional variant thereof, that specifically binds PD1.

In some embodiments, the fusion protein is administered prior to administration of the antibody, or functional fragment or functional variant thereof, that specifically binds PD1. In some embodiments, the fusion protein is administered at least 30 minutes, 1 hour, 3 hours, 6 hours, 12 hours, 24 hours, 48 hours, 72 hours, 4 days, 5 days, 6 days, 7 days, or 14 days prior to administration of the antibody, or functional fragment or functional variant thereof, that specifically binds PD1.

In some embodiments, the fusion protein is administered after administration of the antibody, or functional fragment or functional variant thereof, that specifically binds PD1. In some embodiments, the fusion protein is administered at least 30 minutes, 1 hour, 3 hours, 6 hours, 12 hours, 24 hours, 48 hours, 72 hours, 4 days, 5 days, 6 days, 7 days, or 14 days after administration of the antibody, or functional fragment or functional variant thereof, that specifically binds PD1.

### Exemplary Cancers

In some embodiments, the cancer is metastatic. In some embodiments, the cancer is recurrent. In some embodiments, the cancer is metastatic and recurrent. In some embodiments, the cancer is refractory to the approved standard of care. In some embodiments, the cancer is refractory to at least one approved standard of care. In some embodiments, the cancer is refractory to at least all approved standard of care therapeutics.

In some embodiments, the cancer is EGFR-driven. In some embodiments, the cancer is a solid tumor. In some embodiments, the cancer is a hematological malignancy. In some embodiments, the cancer is selected from the group consisting of breast cancer, anal cancer, pancreatic cancer, thyroid cancer, liver cancer, ovarian cancer, lung cancer, skin cancer, brain cancer, spinal cord cancer, head cancer, neck cancer, and head and neck cancer.

In some embodiments, the cancer is head and neck cancer. In some embodiments, the cancer is head and neck squamous cell carcinoma (HNSCC). In some embodiments, the cancer is recurrent HNSCC. In some embodiments, the cancer is metastatic HNSCC. In some embodiments, the cancer is metastatic and recurrent HNSCC. In some embodiments, the cancer is anal canal. In some embodiments, the cancer is squamous cell carcinoma of anal canal (SCCAC). In some embodiments, the cancer is recurrent SCCAC. In some embodiments, the cancer is metastatic SCCAC. In some embodiments, the cancer is metastatic and recurrent SCCAC.

### Dosing Regimens and Schedules

### Anti-PD1 Antibodies

In some embodiments, the antibody, or functional fragment or functional variant thereof, that specifically binds PD1 is administered to the subject having cancer at a therapeutically effective dose. In some embodiments, the antibody, or functional fragment or functional variant thereof, that specifically binds PD1 is administered to the subject having cancer at a fixed dose. In some embodiments, the antibody, or functional fragment or functional variant thereof, that specifically binds PD1 is administered to the subject having cancer at a flat dose. In some embodiments, the antibody, or functional fragment or functional variant thereof, that specifically binds PD1 is administered to the subject having cancer at a weight based dose.

In some embodiments, the antibody, or functional fragment or functional variant thereof, that specifically binds PD1 is administered to the subject having cancer at a dose from about 100 mg to 500 mg, 100 mg to 400 mg, 100 mg to 300 mg, or 100 mg to 200 mg. In some embodiments, the antibody, or functional fragment or functional variant thereof, that specifically binds PD1 is administered to the subject having cancer at a dose of about 100 mg, 200 mg, 300 mg, 400 mg, or 500 mg. In some embodiments, the antibody, or functional fragment or functional variant thereof, that specifically binds PD1 is administered to the subject having cancer at a dose of about 200 mg. In some embodiments, the antibody, or functional fragment or functional variant thereof, that specifically binds PD1 is administered to the subject having cancer at a dose of about 300 mg.

In some embodiments, the antibody, or functional fragment or functional variant thereof, that specifically binds PD1 is administered to the subject having cancer about every 1, 2, 3, 4, 5, 6, 7, or 8 weeks. In some embodiments, the antibody, or functional fragment or functional variant thereof, that specifically binds PD1 is administered to the subject having cancer about every 1 week. In some embodiments, the antibody, or functional fragment or functional variant thereof, that specifically binds PD1 is administered to the subject having cancer about every 2 weeks. In some embodiments, the antibody, or functional fragment or functional variant thereof, that specifically binds PD1 is administered to the subject having cancer about every 3 weeks.

In some embodiments, the antibody, or functional fragment or functional variant thereof, that specifically binds PD1 is pembrolizumab. In some embodiments, pembrolizumab is administered to the subject at a dose of from about 100 mg to 500 mg, 100 mg to 400 mg, 100 mg to 300 mg, or 100 mg to 200 mg. In some embodiments, pembrolizumab is administered at a dose of about 100 mg, 200 mg, 300 mg, 400 mg, or 500 mg. In some embodiments, pembrolizumab is administered at a dose of about 200 mg. In some embodiments, pembrolizumab is administered at a dose of about 300 mg. In some embodiments, pembrolizumab is administered at a dose of about 400 mg.

In some embodiments, pembrolizumab is administered to the subject about every 1, 2, 3, 4, 5, 6, 7, or 8 weeks. In some embodiments, pembrolizumab is administered to the subject about every 2 weeks. In some embodiments, pembrolizumab is administered to the subject about every 3 weeks. In some embodiments, pembrolizumab is administered to the subject about every 4 weeks. In some embodiments, pembrolizumab is administered to the subject about every 5 weeks. In some embodiments, pembrolizumab is administered to the subject about every 6 weeks.

In some embodiments, pembrolizumab is administered to the subject at a dose of about 200 mg every 3 weeks. In some embodiments, pembrolizumab is administered to the subject at a dose of about 400 mg every 6 weeks.

In some embodiments, the antibody, or functional fragment or functional variant thereof, that specifically binds PD1 is nivolumab. In some embodiments, nivolumab is administered to the subject at a dose of from about 100 mg to 500 mg, 100 mg to 400 mg, 100 mg to 300 mg, or 100 mg to 200 mg. In some embodiments, nivolumab is administered at a dose of about 100 mg, 200 mg, 300 mg, 400 mg, or 500 mg. In some embodiments, nivolumab is administered at a dose of about 200 mg. In some embodiments, nivolumab is administered at a dose of about 240 mg. In some embodiments, nivolumab is administered at a dose of about 300 mg. In some embodiments, nivolumab is administered at a dose of about 360 mg. In some embodiments, nivolumab is administered at a dose of about 400 mg. In some embodiments, nivolumab is administered at a dose of about 480 mg. In some embodiments, nivolumab is administered at a dose of about 500 mg.

In some embodiments, nivolumab is administered to the subject at a dose of from about 1 mg/kg to 5 mg/kg, 1 mg/kg to 4 mg/kg, 1 mg/kg to 3 mg/kg, or 1 mg/kg to 2 mg/kg. In some embodiments, nivolumab is administered at a dose of about 1 mg/kg, 2 mg/kg, 3 mg/kg, 4 mg/kg, or 5 mg/kg. In some embodiments, nivolumab is administered at a dose of about 1 mg/kg. In some embodiments, nivolumab is administered at a dose of about 2 mg/kg. In some embodiments, nivolumab is administered at a dose of about 3 mg/kg.

In some embodiments, nivolumab is administered to the subject about every 1, 2, 3, 4, 5, 6, 7, or 8 weeks. In some embodiments, nivolumab is administered to the subject about every 2 weeks. In some embodiments, nivolumab is administered to the subject about every 3 weeks. In some embodiments, nivolumab is administered to the subject about every 4 weeks. In some embodiments, nivolumab is administered to the subject about every 5 weeks. In some embodiments, nivolumab is administered to the subject about every 6 weeks.

In some embodiments, nivolumab is administered to the subject at a dose of about 240 mg every 2 weeks. In some embodiments, nivolumab is administered to the subject at a dose of about 480 mg every 4 weeks. In some embodiments, nivolumab is administered at a dose of about 360 mg every 3 weeks. In some embodiments, nivolumab is administered to the subject at a dose of about 1 mg/kg every 3 weeks. In some embodiments, nivolumab is administered to the subject at a dose of about 3 mg/kg every 2 weeks.

### Fusion Proteins

In some embodiments, the fusion protein (i.e. fusion protein that comprises a targeting moiety and an immunomodulatory moiety, wherein: i) said targeting moiety specifically binds epidermal growth factor receptor (EGFR); and (ii) said immunomodulatory moiety comprises an amino acid sequence of the extracellular domain of transforming growth factor-beta receptor II (TGFβRII)), is administered to the subject having cancer at a therapeutically effective dose. In some embodiments, the fusion protein is administered to the subject having cancer at a fixed dose. In some embodiments, the fusion protein is administered to the subject having cancer at a flat dose. In some embodiments, the fusion protein is administered to the subject having cancer at a weight based dose.

In some embodiments, the fusion protein is administered to the subject at a dose from about 50 mg to 2000 mg, 100 mg to 2000 mg, 150 mg to 2000 mg, 200 mg to 2000 mg, 300 mg to 2000 mg, 400 mg to 2000 mg, 500 mg to 2000 mg, 600 mg to 2000 mg, 700 mg to 2000 mg, 800 mg to 2000 mg, 9000 mg to 2000 mg, 1000 mg to 2000 mg, 1500 mg to 2000 mg, 50 mg to 100 mg, 50 mg to 500 mg, 50 mg to 400 mg, 50 mg to 300 mg, 50 mg to 200 mg, 50 mg to 100 mg, 100 mg to 500 mg, 100 mg to 400 mg, 100 mg to 300 mg, or 100 mg to 200 mg. In some embodiments, the fusion protein is administered to the subject at a dose of from about 200 mg to 2000 mg. In some embodiments, the fusion protein is administered to the subject at a dose of about 50 mg, 60 mg, 64 mg, 100 mg, 150 mg, 200 mg, 240 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900, or 2000 mg. In some embodiments, the fusion protein is administered to the subject at a dose of about 64 mg, 240 mg, 800 mg, or 1600 mg. In some embodiments, the fusion protein is administered to the subject at a dose of about 64 mg. In some embodiments, the fusion protein is administered to the subject at a dose of about 240 mg. In some embodiments, the fusion protein is administered to the subject at a dose of about 800 mg. In some embodiments, the fusion protein is administered to the subject at a dose of about 1600 mg.

In some embodiments, the fusion protein is administered to the subject every 1, 2, 3, 4, 5, or 6 weeks. In some embodiments, the fusion protein is administered to the subject every week. In some embodiments, the fusion protein is administered to the subject every 2 weeks. In some embodiments, the fusion protein is administered to the subject every 3 weeks. In some embodiments, the fusion protein is administered to the subject every 4 weeks. In some embodiments, the fusion protein is administered to the subject 5 weeks. In some embodiments, the fusion protein is administered to the subject every 6 weeks.

### Kits

In one aspect, provided herein are kits comprising an anti-PD-1 antibody and a fusion protein described herein for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a cancer, the kit comprising: (a) a dosage of an anti-PD-1 antibody; and (b) a dosage of a fusion protein described herein and (c) instructions for using the anti-PD-1 antibody and the fusion protein in any of the combination therapy methods disclosed herein. In certain embodiments, the anti-PD-1 antibody, the fusion protein can be co-packaged in unit dosage form. In certain embodiments for treating human patients, the kit comprises an anti-human PD-1 antibody disclosed herein, e.g., pembrolizumab.

The present invention is further illustrated by the following examples which should not be construed as further limiting. The contents of all references cited throughout this application are expressly incorporated herein by reference.

### EXAMPLES

### Example 1. Evaluation of the Anti-Cancer Activity of BCA101 in Combination with an Anti-PD1 Antibody in huNOG-EXL Mice Bearing Subcutaneous PC-3 Tumor Xenograft

The objective of this study was to evaluate the anti-cancer activity of BCA101 alone and in combination with anti-PD1 antibody pembrolizumab in vivo.

### Materials and Methods

### Mice

The study utilized immunodeficient hGM-CSF/hIL3 transgenic-NOG mice which upon engraftment with human hematopoietic stem cells, exhibit in a human-like immune system (lymphoid & myeloid lineage of human origin). This model enabled the study of key innate mechanisms involved in the efficacy of immuno-therapy related agents and provided a suitable model for establishment of human xenografts.

Male hGM-CSF/hIL3 NOG mice were obtained from Taconic Biosciences. hGM-CSF/hIL3 NOG mice engrafted with human CD34+ hematopoietic stem cells (HSCs) stably developed extensive cell lineages as early as 6 to 8 weeks' post-injection. Both myeloid and lymphoid lineage cells were present in peripheral blood, bone marrow, thymus and spleen and non-lymphoid tissue including lung and liver. huNOG-EXL mice with greater than 25% hCD45+ in peripheral blood were used. The age of the mice at the start of each study was 13-14 weeks, with a mean body weight of the animals per group of approximately 26 g.

### Preparation and Subcutaneous Injection of Tumor Cells

All procedures were performed in a laminar flow hood following sterile techniques. PC-3 (Human prostate adenocarcinoma) cells with a viability of >90% were utilized. Around 5×10⁶ cells were re-suspended in 2001A1 of serum free media containing 50% of matrigel kept in ice.

PC-3 cell line was propagated in male huNOG-EXL mice by injecting the cells subcutaneously in the right flank region of the mice. The implanted area was monitored for tumor growth. Once the tumor attained palpable stage and required volume, the mice were randomized based on tumor volume (Mean tumor volume≈119 mm³) and dosing was initiated. The study schedule is described in Table 6.

TABLE 6
Study Schedule

| | | | Tumor | | |
|---|---|---|---|---|---|
| | | | Volume | | |
| | | | (at | Dose, | |
| | | | initiation | Route | Treat- |
| | No. of | Tumor | of | and Dosing | ment |
| Groups | Animals | Type | study) | Schedule | Period |
| Group I | 10 | PC-3 | ~119 | 8.1 mg/kg; | 21 |
| Isotype | | | mm³ | intraperitoneal; | days |
| Control | | | | Biweekly × | |
| | | | | 6 doses | |
| Group II | 10 | PC-3 | ~119 | 8.1 mg/kg; | 21 |
| Cetuximab | | | mm³ | intraperitoneal; | days |
| | | | | Biweekly × | |
| | | | | 6 doses | |
| Group III | 10 | PC-3 | ~119 | 10 mg/kg; | 21 |
| BCA101 | | | mm³ | intraperitoneal; | days |
| | | | | Biweekly × | |
| | | | | 6 doses | |
| Group IV | 10 | PC-3 | ~119 | 10 mg/kg; | 21 |
| Anti-PD1 | | | mm³ | intraperitoneal; | days |
| (pembrolizumab) | | | | Q5D × 5 doses | |
| Group V | 10 | PC-3 | ~119 | BCA101: | 21 |
| BCA101 + | | | mm³ | 10 mg/kg; | days |
| Anti-PD1 | | | | intraperitoneal; | |
| (pembrolizumab) | | | | Biweekly × | |
| | | | | 6 doses | |
| | | | | Anti-PD1: | |
| | | | | 10 mg/kg; | |
| | | | | intraperitoneal; | |
| | | | | Q5D × 5 doses | |

### Formulation of Antibody Doses

Required quantities of human IgG1 isotype control, cetuximab, BCA101, and pembrolizumab were diluted in diluent buffer to obtain an appropriate working concentration to deliver the intended dose. The dose volume of 10 mL/kg intraperitoneally was maintained for all the animals. Whereas, in combination treatment (Group V) on day 0, dose volume of 5 mL/kg was used for both BCA101 and pembrolizumab dosing to maintain the total dose volume of 10 mL/kg per mouse.

### Observations

### Body Weight and Clinical Sign

Individual body weight was measured every third day during experimental period. The % change in body weight of individual mice was calculated and recorded. Mice were observed for visible clinical sign and recorded every third day during experimental period.

### Tumor Volume

The tumor volume was determined by two-dimensional measurement of length (L) and width (W) of the tumor with a digital Vernier caliper on the day of randomization (Day 0) and then every third day during the experimental period. Tumor volume (TV) was calculated using the following formula: Tumor Volume (mm3)=(L×W²)/2, wherein L=length (mm) and W=width (mm). Mean, Standard Deviation (SD) or Standard Error of Mean (SEM) were calculated for individual groups.

### Antitumor Activity

Antitumor activity was evaluated as maximum tumor growth inhibition versus the isotype control group. Data evaluation was performed using standard calculations in Microsoft Excel.

### Test/Control Value in % (% T/C)

Tumor inhibition on a particular day (T/C in %) was calculated by using the below formula: % T/C Day X=((Mean TV of test group on day x−Mean TV of test group on day 0)/(Mean TV of control group on day x−Mean TV of control group on day 0))×100%; wherein TV=tumor volume in mm³. The minimum (or optimum) % T/C value recorded for a particular test group during an experiment represents the maximum antitumor activity for the respective treatment.

### Tumor Growth Inhibition (TGI)

TGI was calculated using the following formula: TGI=(1−T/C)×100%; wherein T=(Mean TV of the test group on Day X−Mean TV of the test group on Day 0; and C=(Mean TV of the control group on Day X−Mean TV of the control group on Day 0).

### Statistical Analysis

For the evaluation of the statistical significance of tumor inhibition, a Two-way ANOVA followed by Bonferroni post hoc test was performed using Graph Pad Prism V 8.3.0. p values <0.05 indicate statistically significant differences between groups.

### Necropsy

Based on ethical reasons and tumor end points animals showing tumor necrosis/ulcerated and tumor burden (Tumor volume >1500 mm3) were humanely euthanized on day 21 and necropsy was performed.

### Collection of Samples

On day 21, blood sampling was carried out from remaining animals of all the treatment groups. After collection, the blood was allowed to clot for 20 minutes at room temperature. Further, the serum was separated by centrifugation at 2,000×g for 10 minutes and stored at −80° C. for further use. All animals were humanely euthanized and tumor (as per tumor availability) was harvested and divided into two parts One part of the tumor was snap frozen and the second part was fixed in 10% Neutral buffered formalin (NBF).

### Results

Cetuximab, BCA101, and pembrolizumab were evaluated either as a standalone therapy or as a combination therapy (BCA101+pembrolizumab) for antitumor activity against PC-3 tumor xenografts. Cetuximab, BCA101 and pembrolizumab were administered intraperitoneally at a dose of 8.1, 10 and 10 mg/kg, respectively. Combination therapy of BCA101+pembrolizumab were tested with the same dose and regimen. All animals from all the treatment groups were alive at day 18.A such, day 18 was chosen for efficacy evaluation. Treatment with cetuximab, BCA101, pembrolizumab, and BCA101+pembrolizumab demonstrated 65%, 60%, 48% and 22% of % T/C, respectively against hu IgG1 (Isotype control) treatment group (Table 7).

TABLE 7
Effect of cetuximab, BCA101 and pembrolizumab in huNOG-EXL bearing
PC-3 tumor xenograft.

| | | | | Mean % | |
|---|---|---|---|---|---|
| | | | | Body | |
| | Number | % | Statistical | Weight | |
| | of | Test/Control | Analysis | Change | |
| Group | Animals | Day 18 | Day 18 | Day 18 | Mortality |
| Group I | 10 | NA | — | −10% | 2/10 |
| Isotype Control | | | | | (Day 19-D147-A35 |
| | | | | | and day 21-D147-A2) |
| Group II | 10 | 65 | *** | −9% | — |
| Cetuximab | | | (p < 0.001) | | |
| Group III | 10 | 60 | *** | −11% | 1/10 |
| BCA101 | | | (p < 0.001) | | (Day 19-D147-A38) |
| Group IV | 10 | 48 | *** | −8% | — |
| Anti-PD1 | | | (p < 0.001) | | |
| (pembrolizumab) | | | | | |
| Group V | 10 | 22 | *** | −7% | 2/10 (Day 20-D148-A9 |
| BCA101 + Anti-PD1 | | | (p < 0.001) | | and day 21-D149-A65) |
| (pembrolizumab) | | | | | |

*** Statistically significant (p < 0.001) difference when respective treatment groups were compared with isotype control group. Statistical analysis carried out by Two-way ANOVA followed by Bonferroni post tests using Graph Pad Prism (Version 8.3.0).

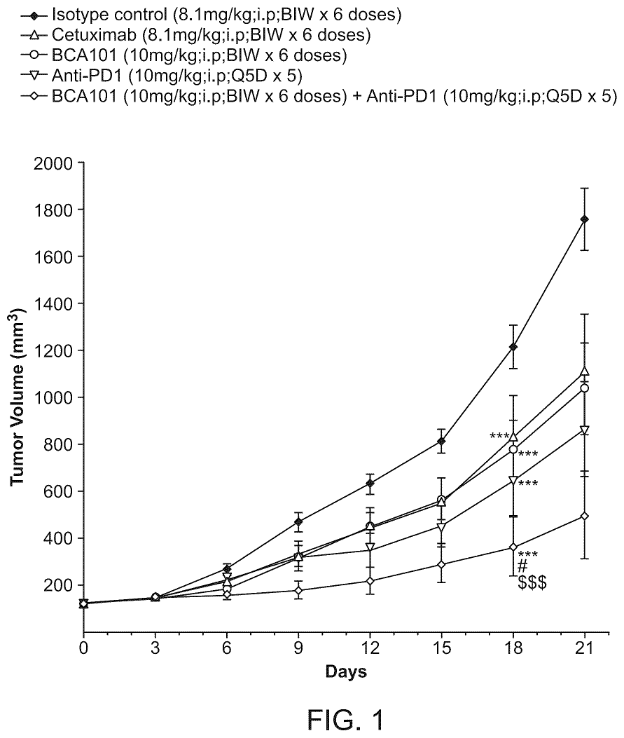

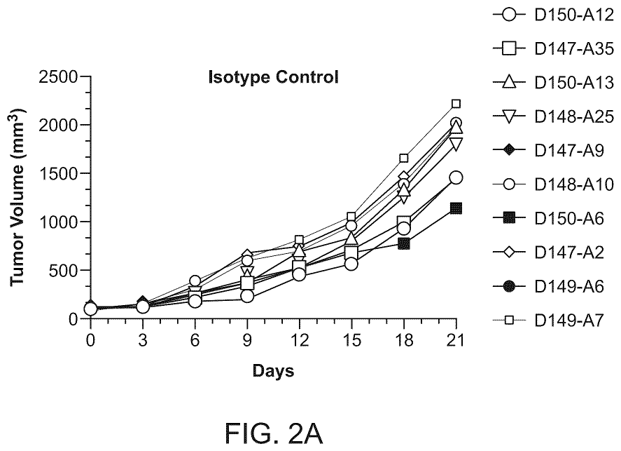

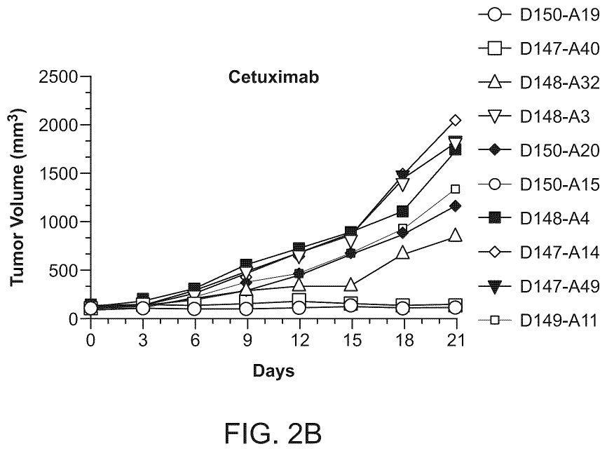

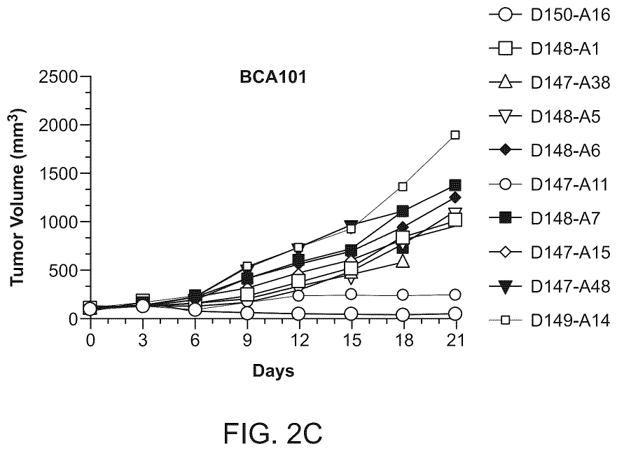

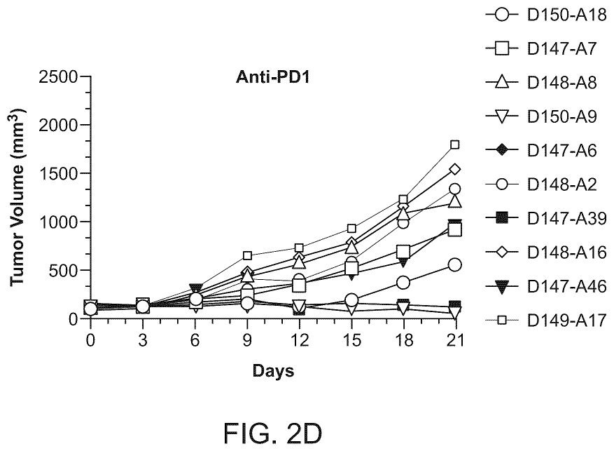

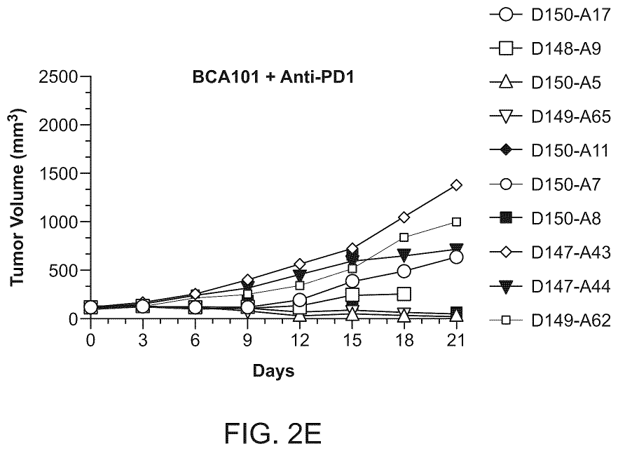

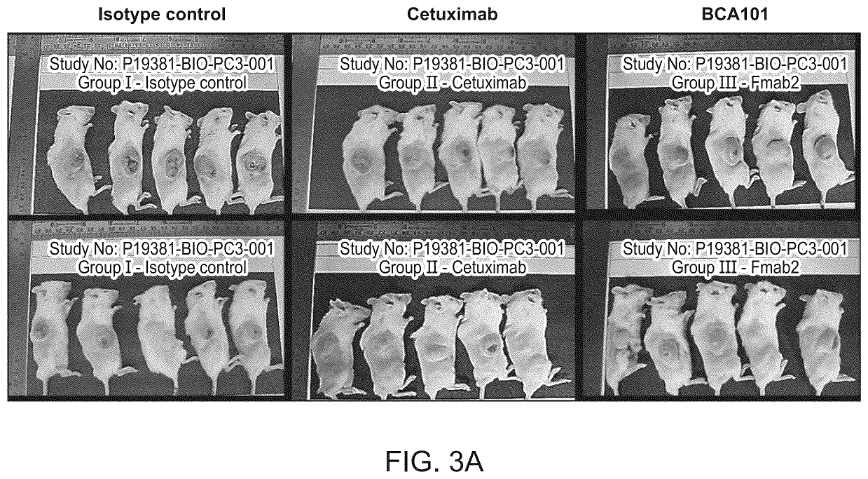

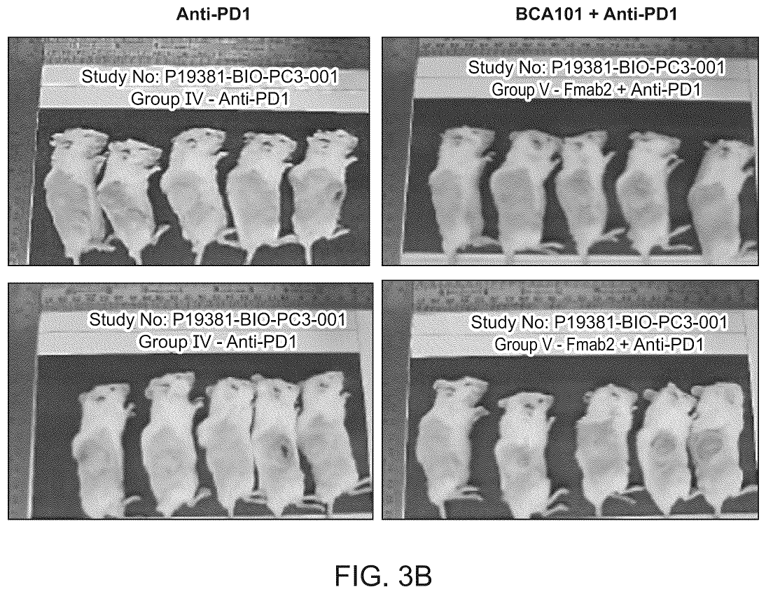

The isotype control group showed progressive tumor growth throughout the experiment with mean tumor volume of 1216±89 mm³ on day 18 (Table 8). The mean tumor volume of cetuximab, BCA101, pembrolizumab and BCA101+pembrolizumab treated groups were recorded as 834±175, 776±127, 647±145 and 362±120 mm³, respectively on day 18 respectively (Table 8). The tumor growth profile and individual tumor growth curve during this period are shown in FIG. 1 and FIGS. 2A-2E, respectively. Animals bearing PC-3 tumors were photographed on day 19 (FIG. 3A and FIG. 3B).

TABLE 8
Mean tumor volume of animals bearing PC-3 xenograft tumors.

| | Mean Tumor Volume (mm³)* | | | | | | | | |
|---|---|---|---|---|---|---|---|---|---|
| Group | Days | 0 | 3 | 6 | 9 | 12 | 15 | 18 | 21 |
| Group I | Mean | 119 | 155 | 270 | 473 | 630 | 814 | 1216 | 1759 |
| Isotype Control | S.E.M. | 3 | 8 | 23 | 47 | 41 | 51 | 89 | 131 |
| Group II | Mean | 119 | 143 | 220 | 333 | 450 | 551 | 834 | 1113 |
| Cetuximab | S.E.M. | 4 | 8 | 23 | 52 | 75 | 99 | 175 | 242 |
| Group III | Mean | 119 | 147 | 186 | 316 | 438 | 563 | 776 | 1041 |
| BCA101 | S.E.M. | 3 | 5 | 20 | 49 | 69 | 90 | 127 | 193 |
| Group IV | Mean | 119 | 149 | 225 | 317 | 351 | 452 | 647 | 862 |
| Anti-PD1 | S.E.M. | 3 | 4 | 20 | 54 | 74 | 96 | 145 | 200 |
| (pembrolizumab) | | | | | | | | | |
| Group V | Mean | 119 | 143 | 159 | 180 | 218 | 290 | 362 | 497 |
| BCA101 + Anti-PD1 | S.E.M. | 2 | 4 | 22 | 36 | 57 | 82 | 120 | 185 |
| (pembrolizumab) | | | | | | | | | |

*Values are expressed as mean of 8-10 animals in each group.

The % TGI values for cetuximab, BCA101, pembrolizumab and BCA101+pembrolizumab groups were calculated as 35%, 40%, 52% and 78% (Day 18), respectively (Table 9) with respect to the isotype control group. The treatment groups cetuximab, BCA101, pembrolizumab and BCA101+pembrolizumab showed significant decrease in the tumor growth (p<0.001; day 18) as compared to isotype control group. Partial tumor growth regression was observed in BCA101 (1/10 mouse), pembrolizumab (2/10 mice) and BCA101+pembrolizumab (4/10 mice) treatment groups. Based on tumor end points (tumor burden; tumor volume >1500 mm³) and ethical reasons (tumor necrosis/ulceration), the study was terminated on day 21 and all animals were humanely euthanized.

TABLE 9
Percentage tumor growth inhibition (% TGI) by delta of test
compounds in huNOG-EXL mice bearing PC-3 tumor xenograft.

| | % Tumor Growth Inhibition (% TGI) by delta | | | | | | | |
|---|---|---|---|---|---|---|---|---|
| Group | Day 0 | Day 3 | Day 6 | Day 9 | Day 12 | Day 15 | Day 18 | Day 21 |
| Group II | 0 | 35 | 34 | 40 | 35 | 38 | 35 | 39 |
| Cetuximab | | | | | | | | |
| Group III | 0 | 23 | 56 | 45 | 38 | 36 | 40 | 44 |
| BCA101 | | | | | | | | |
| Group IV | 0 | 17 | 30 | 44 | 55 | 52 | 52 | 55 |
| Anti-PD1 | | | | | | | | |
| (pembrolizumab) | | | | | | | | |
| Group V | 0 | 33 | 73 | 83 | 81 | 75 | 78 | 77 |
| BCA101 + Anti-PD1 | | | | | | | | |
| (pembrolizumab) | | | | | | | | |

### Mortality and Body Weight Changes

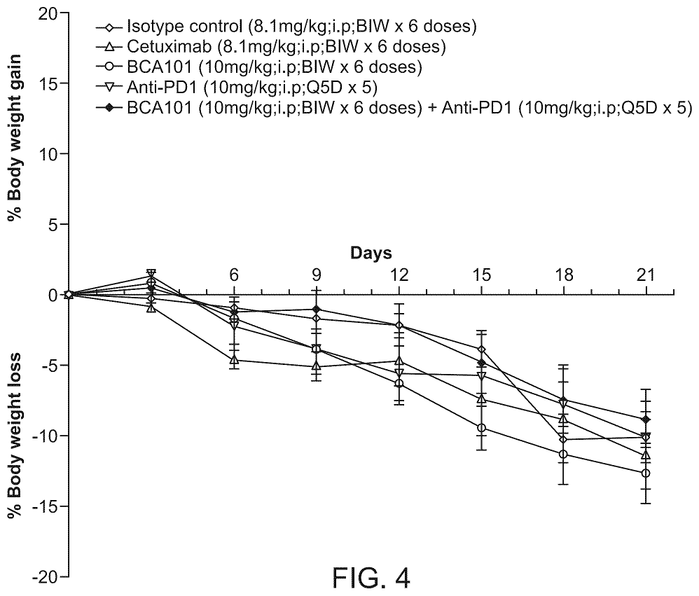

Animal mortalities were observed from isotype control (Day 19-D147-A35 and day 21-D147-A2), BCA101 (Day 19-D147-A38), combination treatment BCA101+pembrolizumab (Day 20-D148-A9 and day 21-D149-A65) during the experimental period. There were no visible clinical signs of abnormal behavior observed in any of the treatment groups. Moreover, gradual decrease in body weight was observed in all the treatment groups. At the end of the study on day 21, severe weight loss (greater than −10%) was observed in all the treatment animals. The percentage change in body weight of animals for all the groups are summarized in Table 10 and FIG. 4.

TABLE 10
Mean body weight and % change in body weight
of animals during PC-3 Xenograft study.

| | Mean Body Weight (g) | | | | | | | | |
|---|---|---|---|---|---|---|---|---|---|
| Group | Days | 0 | 3 | 6 | 9 | 12 | 15 | 18 | 21 |
| Group I | Mean | 25.9 | 25.8 | 25.6 | 25.4 | 25.2 | 24.8 | 23.1 | 22.8 |
| Isotype Control | S.E.M. | 1.0 | 0.9 | 0.9 | 0.8 | 0.8 | 0.8 | 0.7 | 0.6 |
| | % BWC | 0.0 | −0.3 | −1.0 | −1.8 | −2.2 | −3.8 | −10.3 | −10.2 |
| Group II | Mean | 26.9 | 26.7 | 25.7 | 25.5 | 25.6 | 24.8 | 24.4 | 23.8 |
| Cetuximab | S.E.M. | 1.1 | 1.1 | 1.0 | 1.0 | 1.0 | 0.9 | 1.0 | 0.9 |
| | % BWC | 0.0 | −0.8 | −4.6 | −5.1 | −4.6 | −7.4 | −8.8 | −11.4 |
| Group III | Mean | 26.9 | 27.0 | 26.4 | 25.8 | 25.2 | 24.3 | 23.8 | 23.7 |
| BCA101 | S.E.M. | 0.7 | 0.6 | 0.6 | 0.7 | 0.6 | 0.7 | 0.8 | 0.9 |
| | % BWC | 0.0 | 0.6 | −1.7 | −4.0 | −6.3 | −9.5 | −11.4 | −12.7 |
| Group IV | Mean | 25.1 | 25.4 | 24.5 | 24.1 | 23.6 | 23.6 | 23.1 | 22.5 |
| Anti-PD1 | S.E.M. | 0.8 | 0.7 | 0.9 | 0.9 | 0.8 | 0.8 | 0.9 | 0.9 |
| (pembrolizumab) | % BWC | 0.0 | 1.2 | −2.3 | −3.9 | −5.6 | −5.8 | −7.8 | −10.1 |
| Group V | Mean | 25.2 | 25.4 | 24.8 | 24.8 | 24.6 | 23.9 | 23.2 | 23.3 |
| BCA101 + Anti-PD1 | S.E.M. | 0.8 | 0.8 | 0.6 | 0.5 | 0.5 | 0.5 | 0.6 | 0.5 |
| (pembrolizumab) | % BWC | 0.0 | 0.8 | −1.3 | −1.1 | −2.1 | −4.9 | −7.5 | −8.9 |

* % BWC = Body weight change with respect to initial body weight of animals.
**Values are expressed as mean of 8-10 animals in each group.

### CONCLUSIONS

In this PC-3 tumor xenograft model, treatment with cetuximab, BCA101, pembrolizumab and BCA101+pembrolizumab at given dose and regimen showed significant reduction in tumor growth compared to isotype control group. Among the tested dose and regimen, combination treatment (BCA101+pembrolizumab) exhibited maximum tumor growth inhibition with more number of partial tumor regression (4/10 mice) when compared to standalone treatment of pembrolizumab (partial regression-2/10 mice) and BCA101 (partial regression-1/10 mouse) followed by cetuximab (no partial tumor regression). Severe body weight loss might be attributed greatly to the tumor burden in isotype control and tested dose of treatment groups. In conclusion, BCA101 is a candidate for cancer treatment and might exhibit better efficacy in combination with immune-check point inhibitors (e.g., anti-PD1 antibodies) against various xenograft models.

### Example 2. A Phase 1a/1b Dose Escalation and Cohort Expansion Study of Safety and Tolerability of BCA101 Alone and in Combination with Anti-PD1 Antibody (Pembrolizumab) in Patients with EGFR-Driven Advanced Solid Tumors

Generally, this study aims to evaluate the safety, tolerability, PK, pharmacodynamics, and efficacy of BCA101 alone and in combination with pembrolizumab in patients with EGFR-driven advanced solid tumors. This is a Phase 1/1b, open-label study, which consists of dose escalation parts (Part A) followed by expansion cohorts (Part B) for both single agent BCA101 and combination BCA101 plus pembrolizumab.

### Objectives

The primary objective of the dose escalation (Part A) of the study is to 1) assess the safety and tolerability of single agent BCA101 in patients with select EGFR-driven advanced solid tumors refractory to standard of care or for whom no standard of care is available; 2) assess the safety and tolerability of BCA101 in combination with pembrolizumab in patients with either squamous cell carcinoma of the head and neck (HNSCC) or squamous cell carcinoma of the anal canal (SCCAC) whose tumors are refractory to standard of care or for whom no standard of care is available; and 3) identify dose limiting toxicities (DLTs) during the first cycle of treatment with BCA101 monotherapy or the combination of BCA101 and pembrolizumab. Patients will be enrolled as per a sequential “3+3” design.

Once the maximum tolerated dose (MTD)/recommended dose (RD) is determined, the primary objective of dose expansion (Part B) is to further assess 1) the safety and tolerability of single agent BCA101 in patients with select cancers; and 2) the safety and tolerability of BCA101 in combination with pembrolizumab in patients with HNSCC and SCCAC. The patient cohorts for assessment of single agent BCA101 include 1) PD-L1 negative, EGFR-amplified Squamous Cell Lung Cancer (SqCLC); 2) RAS wild-type, microsatellite stable Colorectal Carcinoma (RAS wt, MSS CRC); 3) EGFR-amplified Triple Negative Breast Cancer; and 4) any solid tumor with either a KRAS G12D or G13D mutation.

BCA101 will be administered intravenously every 7 days at the MTD or RD based upon the results from Part A of the study. Pembrolizumab will be administered according to approved product label for use in the specific indication. The decision to proceed with each cohort will be based on the review of the cumulative safety, PK, clinical data, and any PD data, if available, and will aim to ensure that the risk-benefit ratio of the MTD or RD justifies enrollment of patients into an expansion cohort. Patients will continue with weekly infusions until disease progression, unacceptable toxicity, withdrawal of consent by the patient, or if the investigator considers it is in the best interest of a patient to discontinue treatment with the study drug.

The secondary objectives of this study are: 1) determine objective response rate in each part of the study, per RECIST v1.1 and iRECIST; 2) determine clinical benefit rate in each part of the study, per RECIST v1.1 and iRECIST; 3) determine progression free survival (PFS) in each part of the study, per RECIST v1.1 and iRECIST; 4) determine duration of response in each part of the study, per RECIST v1.1 and iRECIST; 5) determine survival rates in each part of the study; 6) AUC of BCA101 and pembrolizumab; 7) Cmax of BCA101 and pembrolizumab; 8) Tmax of BCA101 and pembrolizumab; 9) Concentration vs time profile of BCA101 and pembrolizumab; 10) Half-life of BCA101 and pembrolizumab; and 11) immunogenicity of BCA101 and pembrolizumab through the incidence and titer of anti-drug-antibodies.

Exploratory objectives of this study are to examine the pharmacodynamic markers and biomarkers for BCA101. Exploratory serum endpoints include, the levels of TGFβ1, TGFβ2, TGFβ3, soluble EGFR, VEGF, and other relevant cytokines, interleukins and chemokines. Exploratory blood cell endpoints include, the immunophenotyping by flow cytometry for multiparametric immune profiling of peripheral blood. Exploratory tumor tissue endpoints include, an analysis of archival, pre- and posttreatment biopsy samples for whole exome sequencing and next generation sequencing of DNA; immunohistochemistry for relevant EGFR and TGFβ signaling pathway markers, tumor infiltrating immune cells and other relevant markers.

### Investigational Plan

### Overall Design and Plan of the Study

This is a Phase 1/1b, open-label study, which consists of dose escalation parts (Part A) followed by expansion cohorts (Part B) for both single agent BCA101 and combination BCA101 plus pembrolizumab.

### Dose Escalation (Part A)

Single agent BCA101 will be administered to patients via intravenous infusion weekly at a dose of 64 mg, 240 mg, 800 mg, or 1600 mg. Patients with the following tumor types will be eligible: 1) Squamous Cell Lung Cancer (SqCLC) 2) Squamous Cell Carcinoma of the Head and Neck (HNSCC) 3) RAS wild-type microsatellite stable Colorectal Carcinoma (RAS WT MSS CRC) 4) Triple Negative Breast Cancer (TNBC) 5) Chordoma 6) Squamous Cell Carcinoma of the Anal Canal (SCCAC) 7) Uveal Melanoma 8) Glioblastoma (GBM) 9) Gastric Cancer 10) Any solid tumor with a KRAS G12D or G13D mutation 11) Any solid tumor with EGFR amplification 12) Epithelial Ovarian Cancer 13) Hepatocellular Carcinoma (HCC) 14) Anaplastic Thyroid Cancer (ATC) 15) Pancreatic Cancer 16) Other EGFR-driven advanced solid tumors (if there is compelling data or evidence to enroll a patient with a tumor type other than those listed in 1-15, the treating physician may discuss the patient with the Sponsor to determine eligibility).

Combination BCA101 and pembrolizumab will be administered to patients via intravenous infusion every 3 weeks. Patients with the following tumor types will be eligible: HNSCC and SCCAC.

Cohort expansion (Part B)

Patients must have histologically or cytologically confirmed EGFR-driven, advanced solid tumor refractory to current standard of care therapy. Patients with the following tumor types will be eligible for single agent BCA101 therapy: PD-L1 negative, and EGFR-amplified SqCLC RAS WT MSS CRC EGFR-amplified TNBC, any solid tumor with a KRAS G12D or G13D mutation.

Patients with the following tumor types will be eligible for treatment with combination therapy of BCA101 and pembrolizumab:1) HNSCC 2) SCCAC.

### Exclusion Criteria:

Exposure to anti-EGFR antibodies within 4 weeks of the first dose of study drug or any history of treatment with anti-TGFβ therapies.

Prior history of Grade ≥2 intolerance or hypersensitivity reaction to cetuximab or other anti-EGFR therapy or other murine proteins or prior discontinuation of therapy in the setting of toxicity related to treatment.

For Part B only: Prior history of Grade ≥2 intolerance or hypersensitivity reaction to immune checkpoint inhibitors or any history of treatment discontinuation in the setting of toxicity to an immune checkpoint inhibitor.

Pregnant or breastfeeding women.

Any condition requiring systemic treatment with either corticosteroids (>10 mg daily of prednisone or equivalent) or other immunosuppressive medication within 14 days prior to the first dose of study drug, with the exception of topical, intranasal, intrabronchial, or ocular steroids.

Known case of human immunodeficiency virus (HIV), or active hepatitis B (hepatitis B surface antigen; HBsAg) or hepatitis C.

Patients with past hepatitis B virus (HBV) infection or resolved HBV infection (defined as having a negative HBsAg test and a positive antibody to hepatitis B core antigen [anti-HBc] antibody test) are eligible. Patients positive for hepatitis C virus (HCV) antibody are eligible only if polymerase chain reaction is negative for HCV RNA.

What is claimed is:

1. A method of treating cancer in a human subject in need thereof, said method comprising:
a. administering to said subject an antibody, or functional fragment or functional variant thereof, that specifically binds programmed cell death protein 1 (PD1); and
b. administering to said subject a fusion protein that comprises a targeting moiety and an immunomodulatory moiety, wherein: i) said targeting moiety specifically binds epidermal growth factor receptor (EGFR); and (ii) said immunomodulatory moiety comprises an amino acid sequence of the extracellular domain of transforming growth factor-beta receptor II (TGFβRII).

2. The method of claim 1, wherein said antibody, or functional fragment or functional variant thereof, that specifically binds PD1 is a full-length antibody, a single chain variable fragment (scFv), a scFv2, a scFv-Fc, a Fab, a Fab′, a F(ab′)2, or a F(v).

3. The method of claim 1 or 2, wherein said antibody, or functional fragment or functional variant thereof, that specifically binds PD1 inhibits binding of PD1 to PDL1.

4. The method of any one of the preceding claims, wherein said antibody, or functional fragment or functional variant thereof, that specifically binds PD1 inhibits signaling of PD1.

5. The method of any one of the preceding claims, wherein said antibody, or functional fragment or functional variant thereof, that specifically binds PD1 comprises a VH that comprises VH CDR1, VH CDR2, and VH CDR3, wherein
a. VH CDR1 comprises an amino acid sequence at least 95%, 96%, 97%, 98%, 99%, or 100% identical to the amino acid sequence of SEQ ID NO: 1;
b. VH CDR2 comprises an amino acid sequence at least 95%, 96%, 97%, 98%, 99%, or 100% identical to the amino acid sequence of SEQ ID NO: 2; and
c. VH CDR3 comprises an amino acid sequence at least 95%, 96%, 97%, 98%, 99%, or 100% identical to the amino acid sequence of SEQ ID NO: 3.

6. The method of any one of the preceding claims, wherein said antibody, or functional fragment or functional variant thereof, that specifically binds PD1 comprises a VL that comprises a VL CDR1, a VL CDR2, and a VL CDR3, wherein
a. VL CDR1 comprises an amino acid sequence at least 95%, 96%, 97%, 98%, 99%, or 100% identical to the amino acid sequence of SEQ ID NO: 4;
b. VL CDR2 comprises an amino acid sequence at least 95%, 96%, 97%, 98%, 99%, or 100% identical to the amino acid sequence of SEQ ID NO: 5; and
c. VL CDR3 comprises an amino acid sequence at least 95%, 96%, 97%, 98%, 99%, or 100% identical to the amino acid sequence of SEQ ID NO: 6.

7. The method of any one of the preceding claims, wherein said antibody, or functional fragment or functional variant thereof, that specifically binds PD1 comprises a VH that comprises an amino acid sequence at least 95%, 96%, 97%, 98%, 99%, or 100% identical to the amino acid sequence of SEQ ID NO: 7.

8. The method of any one of the preceding claims, wherein said antibody, or functional fragment or functional variant thereof, that specifically binds PD1 comprises a VL that comprises an amino acid sequence at least 95%, 96%, 97%, 98%, 99%, or 100% identical to the amino acid sequence of SEQ ID NO: 8.

9. The method of any one of the preceding claims, wherein said antibody, or functional fragment or functional variant thereof, that specifically binds PD1 comprises a heavy chain region that comprises an amino acid sequence at least 95%, 96%, 97%, 98%, 99%, or 100% identical to the amino acid sequence of SEQ ID NO: 9.

10. The method of any one of the preceding claims, wherein said antibody, or functional fragment or functional variant thereof, that specifically binds PD1 comprises a heavy chain region that comprises an amino acid sequence at least 95%, 96%, 97%, 98%, 99%, or 100% identical to the amino acid sequence of SEQ ID NO: 10.

11. The method of any one of the preceding claims, wherein said antibody, or functional fragment or functional variant thereof, that specifically binds PD1 comprises a light chain region that comprises an amino acid sequence at least 95%, 96%, 97%, 98%, 99%, or 100% identical to the amino acid sequence of SEQ ID NO: 11.

12. The method of any one of the preceding claims, wherein said antibody, or functional fragment or functional variant thereof, that specifically binds PD1 comprises pembrolizumab, nivolumab, cemiplimab, spartalizumab, camrelizumab, tislelizumab, dostarlimab, cetrelimab, pidilizumab, MEDI0680, SSI-361, AMP-224, PDR001, PF-06801591, BGB-A317, TSR-042, AGEN-2034, A-0001, BGB-108, BI-754091, CBT-501, ENUM-003, ENUM-388D4, IBI-308, JNJ-63723283, JS-001, JTX-4014, JY-034, CLA-134, STIA-1110, 244C8, and 388D4, or a functional fragment or functional variant of any of the foregoing.

13. The method of claim 12, wherein said antibody, or functional fragment or functional variant thereof, that specifically binds PD1 comprises pembrolizumab, or a functional fragment or functional variant of any of the foregoing.

14. The method of any one of the preceding claims, wherein said targeting moiety that specifically binds EGFR comprises an antibody or functional fragment or functional variant thereof, that specifically binds EGFR.

15. The method of claim 14, wherein said antibody, or functional fragment or functional variant thereof, that specifically binds EGFR is a full-length antibody, a single chain variable fragment (scFv), a scFv2, a scFv-Fc, a Fab, a Fab′, a F(ab′)2, or a F(v).

16. The method of claim 14 or 15, wherein said antibody, or functional fragment or functional variant thereof, that specifically binds EGFR comprises a VH that comprises VH CDR1, VH CDR2, and VH CDR3, wherein
a. VH CDR1 comprises an amino acid sequence at least 95%, 96%, 97%, 98%, 99%, or 100% identical to the amino acid sequence of SEQ ID NO: 34;
b. VH CDR2 comprises an amino acid sequence at least 95%, 96%, 97%, 98%, 99%, or 100% identical to the amino acid sequence of SEQ ID NO: 35; and
c. VH CDR3 comprises an amino acid sequence at least 95%, 96%, 97%, 98%, 99%, or 100% identical to the amino acid sequence of SEQ ID NO: 36.

17. The method of any one of claims 14-16, wherein said antibody, or functional fragment or functional variant thereof, that specifically binds EGFR comprises a VL that comprises a VL CDR1, a VL CDR2, and a VL CDR3, wherein
a. VL CDR1 comprises an amino acid sequence at least 95%, 96%, 97%, 98%, 99%, or 100% identical to the amino acid sequence of SEQ ID NO: 37;
b. VL CDR2 comprises an amino acid sequence at least 95%, 96%, 97%, 98%, 99%, or 100% identical to the amino acid sequence of SEQ ID NO: 38; and
c. VL CDR3 comprises an amino acid sequence at least 95%, 96%, 97%, 98%, 99%, or 100% identical to the amino acid sequence of SEQ ID NO: 39.

18. The method of any one of claims 14-17, wherein said antibody, or functional fragment or functional variant thereof, that specifically binds EGFR comprises a VH that comprises an amino acid sequence at least 95%, 96%, 97%, 98%, 99%, or 100% identical to the amino acid sequence of SEQ ID NO: 40.

19. The method of any one of claims 14-18, wherein said antibody, or functional fragment or functional variant thereof, that specifically binds EGFR comprises a VL that comprises an amino acid sequence at least 95%, 96%, 97%, 98%, 99%, or 100% identical to the amino acid sequence of SEQ ID NO: 41.

20. The method of any one of claims 14-19, wherein said antibody, or functional fragment or functional variant thereof, that specifically binds EGFR comprises a heavy chain that comprises an amino acid sequence at least 95%, 96%, 97%, 98%, 99%, or 100% identical to the amino acid sequence of SEQ ID NO: 42.

21. The method of any one of claims 14-19, wherein said antibody, or functional fragment or functional variant thereof, that specifically binds EGFR consists of a heavy chain that comprises an amino acid sequence at least 95%, 96%, 97%, 98%, 99%, or 100% identical to the amino acid sequence of SEQ ID NO: 43.

22. The method of any one of claims 14-19, wherein said antibody, or functional fragment or functional variant thereof, that specifically binds EGFR comprises a heavy chain that consists of an amino acid sequence at least 95%, 96%, 97%, 98%, 99%, or 100% identical to the amino acid sequence of SEQ ID NO: 42.

23. The method of any one of claims 14-19, wherein said antibody, or functional fragment or functional variant thereof, that specifically binds EGFR consists of a heavy chain that consists of an amino acid sequence at least 95%, 96%, 97%, 98%, 99%, or 100% identical to the amino acid sequence of SEQ ID NO: 43.

24. The method of any one of claims 14-23, wherein said antibody, or functional fragment or functional variant thereof, that specifically binds EGFR comprises a light chain that comprises an amino acid sequence at least 95%, 96%, 97%, 98%, 99%, or 100% identical to the amino acid sequence of SEQ ID NO: 44.

25. The method of any one of claims 14-23, wherein said antibody, or functional fragment or functional variant thereof, that specifically binds EGFR consists of a light chain that consists of an amino acid sequence at least 95%, 96%, 97%, 98%, 99%, or 100% identical to the amino acid sequence of SEQ ID NO: 44.

26. The method of claim 14, wherein said antibody, or functional fragment or functional variant thereof, that specifically binds EGFR comprises cetuximab or panitumumab, or a functional fragment or functional variant of any of the foregoing.

27. The method of any one of the preceding claims, wherein said immunomodulatory moiety comprises an amino acid sequence at least 95%, 96%, 97%, 98%, 99%, or 100% identical to the amino acid sequence of SEQ ID NO: 56.

28. The method of any one of the preceding claims, wherein said immunomodulatory moiety consists of an amino acid sequence at least 95%, 96%, 97%, 98%, 99%, or 100% identical to the amino acid sequence of SEQ ID NO: 56.

29. The method of any one of the preceding claims, wherein said immunomodulatory moiety is indirectly fused to said targeting moiety.

30. The method of claim 29, wherein said immunomodulatory moiety is indirectly fused to said targeting moiety via a peptide linker.

31. The method of claim 30, wherein said immunomodulatory moiety is indirectly fused to said targeting moiety via a peptide linker of sufficient length such that said immunomodulatory moiety and said targeting moiety can simultaneous bind the respective targets.

32. The method of claim 30 or 31, wherein said linker comprises the amino acid sequence of SEQ ID NO: 57, 58, 59, 60, or 61.

33. The method of claim 30 or 31, wherein said linker comprises the amino acid sequence of SEQ ID NO: 57.

34. The method of claim 30 or 31, wherein said linker consists of the amino acid sequence of SEQ ID NO: 57.

35. The method of any one of claims 1-34, wherein said immunomodulatory moiety is fused to the C terminus of said targeting moiety.

36. The method of any one of claims 1-34, wherein said immunomodulatory moiety is fused to the N terminus of said targeting moiety.

37. The method of claim 1, wherein said targeting moiety is an antibody that comprises a light chain and a heavy chain, and wherein said immunomodulatory moiety is fused to the C terminus of said heavy chain of said targeting moiety.

38. The method of claim 1, wherein said targeting moiety is an antibody that comprises a light chain and a heavy chain, and wherein said immunomodulatory moiety is fused to the C terminus of said light chain of said targeting moiety.

39. The method of claim 1, wherein said targeting moiety is an antibody specifically binds epidermal growth factor receptor (EGFR) that comprises a heavy chain that comprises an amino acid sequence at least 95%, 96%, 97%, 98%, 99%, or 100% identical to the amino acid sequence of SEQ ID NO: 43, and a light chain that comprises an amino acid sequence at least 95%, 96%, 97%, 98%, 99%, or 100% identical to the amino acid sequence of SEQ ID NO: 44, and wherein said immunomodulatory moiety comprises an amino acid sequence at least 95%, 96%, 97%, 98%, 99%, or 100% identical to the amino acid sequence of SEQ ID NO: 56, and wherein the N terminus of said immunomodulatory moiety is fused indirectly through a linker to the C terminus of said heavy chain or said light chain, and wherein said linker comprises an amino acid sequence at least 95%, 96%, 97%, 98%, 99%, or 100% identical to the amino acid sequence of SEQ ID NO: 57.

40. The method of claim 1, wherein said targeting moiety is an antibody specifically binds epidermal growth factor receptor (EGFR) that comprises a heavy chain that comprises an amino acid sequence at least 95%, 96%, 97%, 98%, 99%, or 100% identical to the amino acid sequence of SEQ ID NO: 43, and a light chain that comprises an amino acid sequence at least 95%, 96%, 97%, 98%, 99%, or 100% identical to the amino acid sequence of SEQ ID NO: 44, and wherein said immunomodulatory moiety comprises an amino acid sequence at least 95%, 96%, 97%, 98%, 99%, or 100% identical to the amino acid sequence of SEQ ID NO: 56, and wherein the N terminus of said immunomodulatory moiety is fused indirectly through a linker to the C terminus of said light chain, and wherein said linker comprises an amino acid sequence at least 95%, 96%, 97%, 98%, 99%, or 100% identical to the amino acid sequence of SEQ ID NO: 57.

41. The method of claim 1, wherein said targeting moiety comprises an antibody that comprises a heavy chain comprising an amino acid sequence at least 95%, 96%, 97%, 98%, 99%, or 100% identical to the amino acid sequence of SEQ ID NO: 43; and a light chain comprising an amino acid sequence at least 95%, 96%, 97%, 98%, 99%, or 100% identical to the amino acid sequence of SEQ ID NO: 62.

42. The method of any one of the preceding claims, wherein said cancer is a solid tumor.

43. The method of claim 42, wherein said cancer is selected from the group consisting of breast cancer, anal cancer, pancreatic cancer, thyroid cancer, liver cancer, ovarian cancer, lung cancer, skin cancer, brain cancer, spinal cord cancer, head cancer, neck cancer, and head and neck cancer.

44. The method of claim 43, wherein said cancer is head and neck cancer.

45. The method of claim 44, wherein said cancer is head and neck squamous cell carcinoma (HNSCC).

46. The method of claim 45, wherein said cancer is recurrent HNSCC.

47. The method of claim 45, wherein said cancer is metastatic HNSCC.

48. The method of claim 45, wherein said cancer is recurrent and metastatic HNSCC.

49. The method of claim 43, wherein said cancer is squamous cell carcinoma of anal canal (SCCAC).

50. The method of claim 49, wherein said cancer is recurrent SCCAC.

51. The method of claim 49, wherein said cancer is metastatic SCCAC.

52. The method of claim 49, wherein said cancer is recurrent and metastatic SCCAC.

53. The method of any one of the preceding claims, wherein said antibody, or functional fragment or functional variant thereof, that specifically binds PD1 is administered to said human subject at a dose from about 100 mg to 500 mg, 100 mg to 400 mg, 100 mg to 300 mg, or 100 mg to 200 mg.

54. The method of any one of the preceding claims, wherein said antibody, or functional fragment or functional variant thereof, that specifically binds PD1 is administered to said human subject at a dose of about 100 mg, 200 mg, 300 mg, 400 mg, or 500 mg.

55. The method of any one of the preceding claims, wherein said antibody, or functional fragment or functional variant thereof, that specifically binds PD1 is administered to said human subject at a dose of about 200 mg.

56. The method of any one of claims 53-54, wherein said antibody, or functional fragment or functional variant thereof, that specifically binds PD1 is administered to said human subject at a dose of about 400 mg.

57. The method of any one of the preceding claims, wherein said antibody, or functional fragment or functional variant thereof, that specifically binds PD1 is administered to said human subject every 1, 2, 3, or 4 weeks.

58. The method of claim 57, wherein said antibody, or functional fragment or functional variant thereof, that specifically binds PD1 is administered to said human subject every 3 weeks.

59. The method of any one of claims 1-52, wherein said antibody, or functional fragment or functional variant thereof, that specifically binds PD1 is administered to said human subject at a dose of about 200 mg every 3 weeks.

60. The method of any one of claims 1-52, wherein said antibody, or functional fragment or functional variant thereof, that specifically binds PD1 is administered to said human subject at a dose of about 400 mg every 6 weeks.

61. The method of any one of the preceding claims, wherein said fusion protein is administered to said human subject at a dose from about 50 mg to 2000 mg, 100 mg to 2000 mg, 150 mg to 2000 mg, 200 mg to 2000 mg, 300 mg to 2000 mg, 400 mg to 2000 mg, 500 mg to 2000 mg, 600 mg to 2000 mg, 700 mg to 2000 mg, 800 mg to 2000 mg, 9000 mg to 2000 mg, 1000 mg to 2000 mg, 1500 mg to 2000 mg, 50 mg to 100 mg, 50 mg to 500 mg, 50 mg to 400 mg, 50 mg to 300 mg, 50 mg to 200 mg, 50 mg to 100 mg, 100 mg to 500 mg, 100 mg to 400 mg, 100 mg to 300 mg, or 100 mg to 200 mg.

62. The method of any one of the preceding claims, wherein said fusion protein is administered to said human subject at a dose from about 200 mg to 2000 mg.

63. The method of any one of the preceding claims, wherein said fusion protein is administered to said human subject at a dose of about 50 mg, 60 mg, 64 mg, 100 mg, 150 mg, 200 mg, 240 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900, or 2000 mg.

64. The method of any one of the preceding claims, wherein said fusion protein is administered to said human subject at a dose of about 64 mg, 240 mg, 800 mg, or 1600 mg.

65. The method of any one of the preceding claims, wherein said fusion protein is administered to said human subject every 1, 2, 3, or 4 weeks.

66. The method of claim 65, wherein said fusion protein is administered to said human subject every week.

67. The method of claim 65, wherein said fusion protein is administered to said human subject 3 weeks.

68. The method of any one of claims 1-65, wherein said fusion protein is administered to said human subject every seven days.

69. The method of any one of the preceding claims, wherein the fusion protein is administered via intravenous injection to said human subject.

70. The method of any one of the preceding claims, wherein said fusion protein is co-administered, administered prior to, or administered after, said antibody, or functional fragment or functional variant thereof, that specifically binds PD1.


SEQUENCE LISTING (67 sequences)

SEQ ID NO: 1 - protein, 5 aa, Artificial Sequence
  Synthetic Construct
NYYMY

SEQ ID NO: 2 - protein, 17 aa, Artificial Sequence
  Synthetic Construct
GINPSNGGTNFNEKFKN

SEQ ID NO: 3 - protein, 11 aa, Artificial Sequence
  Synthetic Construct
RDYRFDMGFDY

SEQ ID NO: 4 - protein, 15 aa, Artificial Sequence
  Synthetic Construct
RASKGVSTSGYSYLH

SEQ ID NO: 5 - protein, 7 aa, Artificial Sequence
  Synthetic Construct
LASYLES

SEQ ID NO: 6 - protein, 9 aa, Artificial Sequence
  Synthetic Construct
QHSRDLPLT

SEQ ID NO: 7 - protein, 120 aa, Artificial Sequence [sequence omitted]

SEQ ID NO: 8 - protein, 111 aa, Artificial Sequence [sequence omitted]

SEQ ID NO: 9 - protein, 447 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 446 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 11 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 5 aa, Artificial Sequence
  Synthetic Construct
NSGMH

SEQ ID NO: 13 - protein, 17 aa, Artificial Sequence
  Synthetic Construct
VIWYDGSKRYYADSVKG

SEQ ID NO: 14 - protein, 4 aa, Artificial Sequence
  Synthetic Construct
NDDY

SEQ ID NO: 15 - protein, 11 aa, Artificial Sequence
  Synthetic Construct
RASQSVSSYLA

SEQ ID NO: 16 - protein, 7 aa, Artificial Sequence
  Synthetic Construct
DASNRAT

SEQ ID NO: 17 - protein, 9 aa, Artificial Sequence
  Synthetic Construct
QQSSNWPRT

SEQ ID NO: 18 - protein, 113 aa, Artificial Sequence [sequence omitted]

SEQ ID NO: 19 - protein, 107 aa, Artificial Sequence [sequence omitted]

SEQ ID NO: 20 - protein, 440 aa, Artificial Sequence
  Synthetic Construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 439 aa, Artificial Sequence
  Synthetic Construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 22 - protein, 214 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 5 aa, Artificial Sequence
  Synthetic Construct
NFGMT

SEQ ID NO: 24 - protein, 17 aa, Artificial Sequence
  Synthetic Construct
GISGGGRDTYFADSVKG

SEQ ID NO: 25 - protein, 8 aa, Artificial Sequence
  Synthetic Construct
WGNIYFDY

SEQ ID NO: 26 - protein, 11 aa, Artificial Sequence
  Synthetic Construct
RASLSINTFLN

SEQ ID NO: 27 - protein, 7 aa, Artificial Sequence
  Synthetic Construct
AASSLHG

SEQ ID NO: 28 - protein, 9 aa, Artificial Sequence
  Synthetic Construct
QQSSNTPFT

SEQ ID NO: 29 - protein, 114 aa, Artificial Sequence
  Synthetic Construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVT

SEQ ID NO: 30 - protein, 107 aa, Artificial Sequence [sequence omitted]

SEQ ID NO: 31 - protein, 444 aa, Artificial Sequence
  Synthetic Construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 443 aa, Artificial Sequence
  Synthetic Construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 33 - protein, 214 aa, Artificial Sequence
  Synthetic Construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 5 aa, Artificial Sequence
  Synthetic Construct
NYGVH

SEQ ID NO: 35 - protein, 16 aa, Artificial Sequence
  Synthetic Construct
VIWSGGNTDYNTPFTS

SEQ ID NO: 36 - protein, 11 aa, Artificial Sequence
  Synthetic Construct
ALTYYDYEFAY

SEQ ID NO: 37 - protein, 11 aa, Artificial Sequence
  Synthetic Construct
RASQSIGTNIH

SEQ ID NO: 38 - protein, 7 aa, Artificial Sequence
  Synthetic Construct
YASESIS

SEQ ID NO: 39 - protein, 9 aa, Artificial Sequence
  Synthetic Construct
QQNNNWPTT

SEQ ID NO: 40 - protein, 119 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 41 - protein, 107 aa, Artificial Sequence
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 42 - protein, 449 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 448 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 214 aa, Artificial Sequence
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 7 aa, Artificial Sequence
  Synthetic Construct
SGDYYWT

SEQ ID NO: 46 - protein, 16 aa, Artificial Sequence
  Synthetic Construct
HIYYSGNTNYNPSLKS

SEQ ID NO: 47 - protein, 9 aa, Artificial Sequence
  Synthetic Construct
DRVTGAFDI

SEQ ID NO: 48 - protein, 11 aa, Artificial Sequence
  Synthetic Construct
QASQDISNYLN

SEQ ID NO: 49 - protein, 7 aa, Artificial Sequence
  Synthetic Construct
DASNLET

SEQ ID NO: 50 - protein, 9 aa, Artificial Sequence
  Synthetic Construct
QHFDHLPLA

SEQ ID NO: 51 - protein, 119 aa, Artificial Sequence [sequence omitted]

SEQ ID NO: 52 - protein, 107 aa, Artificial Sequence [sequence omitted]

SEQ ID NO: 53 - protein, 445 aa, Artificial Sequence
  Synthetic Construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 54 - protein, 444 aa, Artificial Sequence
  Synthetic Construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 55 - protein, 214 aa, Artificial Sequence
  Synthetic Construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 137 aa, Artificial Sequence
  Synthetic Construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 57 - protein, 15 aa, Artificial Sequence
  Synthetic Construct
GGGGSGGGGSGGGGS

SEQ ID NO: 58 - protein, 19 aa, Artificial Sequence
  Synthetic Construct
GGGGSGGGGSGGGGSGGGS

SEQ ID NO: 59 - protein, 23 aa, Artificial Sequence
  Synthetic Construct
GGGGSGGGGSGGGGSGGGSGGGS

SEQ ID NO: 60 - protein, 10 aa, Artificial Sequence
  Synthetic Construct
GGGGSGGGGS

SEQ ID NO: 61 - protein, 5 aa, Artificial Sequence
  Synthetic Construct
GGGGS

SEQ ID NO: 62 - protein, 366 aa, Artificial Sequence
  Synthetic Construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 63 - protein, 600 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 64 - protein, 601 aa, Artificial Sequence
  Synthetic Construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 65 - protein, 366 aa, Artificial Sequence
  Synthetic Construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 66 - protein, 597 aa, Artificial Sequence
  Synthetic Construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 67 - protein, 597 aa, Artificial Sequence
  Synthetic Construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD